(19) United States
(12) Patent Application Publication
(10) Pub. No.: US 2018/0171032 A1
(43) Pub. Date: Jun. 21, 2018

(54) RUBBER-LIKE MATERIAL FOR THE IMMOBILIZATION OF PROTEINS AND ITS USE IN LIGHTING, DIAGNOSIS AND BIOCATALYSIS

(71) Applicant: Fundación IMDEA Materiales, Getafe (ES)

(72) Inventors: Rubén Dario COSTA RIQUELME, Fürth (DE); Uwe SONNEWALD, Möhrendorf (DE); Pedro BRANA COTO, Erlangen (DE); Michael Dominik WEBER, Erlangen (DE); Marlene PRÖSCHEL, Nürnberg (DE)

(21) Appl. No.: 15/737,434

(22) PCT Filed: Jun. 17, 2016
(86) PCT No.: PCT/EP2016/064097

§ 371 Date: Dec. 18, 2017

(30) Foreign Application Priority Data
Jun. 19, 2015 (EP) 15173026.4

(51) Int. Cl. C07K 17/04; C12N 9/96; C07K 17/08; C08J 3/075; C09K 11/06; C09K 11/02; C12Q 1/00; C12Q 1/40; C12Q 1/533; C12Q 1/48; C12N 9/12; C12N 9/26; C12N 9/92; H01L 33/56

(52) U.S. Cl. CPC C07K 17/04; C09K 2211/14; C07K 17/08; C08J 3/075; C09K 11/06; C09K 11/025; C12Q 1/008; C12Q 1/40; C12Q 1/533; C12Y 503/01009; C12Q 1/485; C12Y 207/01001; C12N 9/1205; C12N 9/2431; C12N 9/92; H01L 33/56; C08J 2371/02; C08J 2471/02; C08J 2489/00; C12N 9/96

(57) ABSTRACT

The present invention relates to a process of preparing a rubber-like material containing a protein immobilized therein, as well as a corresponding rubber-like material, the process comprising the steps of (a) mixing a protein, a branched polymer such as trimethylolpropane ethoxylate and a linear polymer such as poly(ethylene oxide) in an aqueous solution to form a gel, and (b) drying the gel to obtain a rubber-like material containing the protein immobilized therein, wherein the branched polymer comprises at least three polymeric branches bound to a central branching unit. The rubber-like material allows the immobilization and stabilization of a wide range of different proteins, including luminescent proteins as well as enzymes, and can particularly advantageously be used as down-converting material for light-emitting diodes (LEDs), for diagnostic applications, and in bioreactors.

The present invention relates to a process of preparing a rubber-like material containing a protein immobilized therein, as well as a corresponding rubber-like material, the process comprising the steps of (a) mixing a protein, a branched polymer such as trimethylolpropane ethoxylate and a linear polymer such as poly(ethylene oxide) in an aqueous solution to form a gel, and (b) drying the gel to obtain a rubber-like material containing the protein immobilized therein, wherein the branched polymer comprises at least three polymeric branches bound to a central branching unit. The rubber-like material allows the immobilization and stabilization of a wide range of different proteins, including luminescent proteins as well as enzymes, and can particularly advantageously be used as down-converting material for light-emitting diodes (LEDs), in diagnostic applications, and in bioreactors.

The immobilization of proteins for biotechnological processes has been heralded as the most cost effective means to circumvent the low operational lifetime of these materials caused by the harsh conditions of industrial processes, as well as storage and application conditions for biocatalysis, diagnosis, lighting, etc. In this field, the support, the nature of the matrix, and the immobilization technique are the key-parameters.

The most commonly used supports are divided into i) organic materials like carboxymethyl-cellulose, starch, collagen, modified sepharose, ion exchange resins, active charcoal, polymers, active membranes, etc. and ii) inorganic materials like silica, clay, metal oxides, diatomaceous earth, hydroxyapatite, ceramic, glass, etc. As such, the selection of the support requires consideration about the affinity for the protein, availability of reactive functional groups, mechanical stability, rigidity, and biocompatibility.

The nature of the matrix and the chemical functionalization of the protein features dictate the immobilization technique ranging from reversible physical adsorption by weak interactions such as hydrogen bonds, hydrophobic interactions, van der Waals forces, mechanical containment, etc. to irreversible chemical approach via covalent interactions between groups attached on the protein surface and the matrix and/or support. In short, there are two principal techniques for immobilization, namely direct adsorption onto the support and entrapment in a network.

The physical adsorption onto any kind of substrates is highly interesting, since the support can be regenerated once the activity is reduced. The main drawbacks involve i) the usual need of functionalization of both the support and the protein, ii) the long periods of fabrication that consists of two steps—soaking and incubation, iii) easy leaching of the protein, and iv) etching of the support due to pH, temperature and/or ionic strength of the buffer.

The entrapment involves the immobilization of proteins into the support or organic networks like fibers, polymers, membranes, etc. This technique reduces the leaching process and increases the stability of the proteins. The typical materials for the organic network consist of mixtures of polyanionic or polycationic polymers and multivalent counter-ion polymers, sol-gels, polymer/sol-gel composites based on alginate, carrageenan, collagen, polyacrylamide, gelatin, silicon rubber, polyurethane, polyvinyl alcohols, etc. The practical use of this method is limited by i) the low accessibility of the protein to the reagents, ii) the ease of deactivation of the protein during the fabrication process, iii) the low loading and/or destruction of the support, iv) the use of harsh conditions for its preparation that typically involve gamma, UV and/or thermal treatments, chemical reactions like cross-linking, etc., v) the need of a careful optimization of each components—prepolymers, photosensitizer, cross-link agents, etc.—for each specific protein, and vi) the difficult processing of the final material in terms of, for example, fabrication of thin films, miniaturization, encapsulation, etc., prohibiting their technological application.

Among the different methods for entrapment, the cross-linking approach is the most popular one. This is made by mixing the prepolymers with photosensitizer and/or cross-link agents together with a protein in solution, which is subsequently gelled by i) exposure to UV-radiation, ii) freezing the polymer-containing monomer solution followed by assisted polymerization by gamma radiation, and iii) chemically initiated polymerization process and further neutralization.

Such methods for the immobilization or entrapment of proteins have been described, e.g., in Mohamad N R et al., Biotechnol Biotechnol Equip. 2015, 29(2), 205-220; Datta S et al., 3 Biotech. 2013, 3(1), 1-9; US 5,763,409; and US 2013/0273531.

However, none of the known protein-based entrapments show elastomeric or rubber-like features that are of importance for technological applications like encapsulation of optoelectronic devices, easy deposition and/or transferring on different 3D substrates for diagnosis, miniaturization process, etc.

In recent years, research in white light-emitting diodes (WLEDs) has become of utmost relevance, since they are heralded as the key solid-state lighting source to replace energy inefficient incandescent light bulbs and environmentally not friendly fluorescent lamps. Up to date, two main strategies have been adopted in the design of WLEDs. The first employs inorganic-based materials, most frequently phosphors of rare-earth elements, as color converters (Park J K et al., Appl Phys Lett. 2004, 84, 1647; Jang H S et al., Appl Phys Lett. 2007, 90, 041906; Xie R-J et al., Nitride Phosphors and Solid-State Lighting, CRC Press, 2011; Tolhurst T M et al., Adv Opt Mater. 2015, 3, 546; Zhang R et al., Laser Photon Rev. 2014, 8, 158). They are robust and long-lived, featuring excellent lighting performances. However, they require rather harsh fabrication conditions—e.g., high temperatures—as well as special electronic configurations to dissipate heat, hampering their implementation into large and/or flexible panels. Furthermore, due to the scarcity of these materials, the device fabrication costs are very high. As a second approach, organic light-emitting diodes (OLEDs) have emerged as the most mature technology for large area lighting applications (Reineke S et al., Rev Mod Phys. 2013, 85, 1245; Volz D et al., Green Chem. 2015, 17, 1988). They can be easily fabricated on flexible substrates, but need a multi-layered structure for high performance. Therefore, the state-of-the-art white OLEDs show a clear trade-off in terms of low-cost production and high performance (Reineke S et al., Rev Mod Phys. 2013, 85, 1245; Volz D et al., Green Chem. 2015, 17, 1988).

The aforementioned limitations have fueled an intense research in hybrid inorganic/organic LED architectures (HLEDs), which combines the best of both approaches (Heliotis G et al., Adv Mater. 2006, 18, 334; Gu E et al., Appl Phys Lett. 2007, 90, 031116; Huyal I O et al., J Mater Chem. 2008, 18, 3568; Kim 0 et al., ACS Nano 2010, 4, 3397; Stupca Metal., J Appl Phys. 2012, 112, 074313; Ban D et al., Phys Status Solidi Curr Top Solid State Phys. 2012, 9, 2594; Jang E-P et al., Nanotechnology 2013, 24, 045607; Findlay N J et al., J Mater Chem C 2013, 1, 2249; Lai C-F et al., Opt Lett. 2013, 38, 4082; Chen J et al., J Mater Sci. 2014, 49, 7391; Kim J-Y, Opt Commun. 2014, 321, 86; Shen P-C et al., Sci Rep. 2014, 4, 5307; Findlay N J et al., Adv Mater. 2014, 26, 7290). HLEDs can combine the excellent lighting performance of inorganic LEDs and the low-cost production and ease of color tunability of the organic compounds used in OLEDs.

In particular, hybrid light-emitting diodes (HLEDs) consist of a high-energy emitting inorganic LED - i.e., emission wavelength from 360 to 470 nm—that is coated with a down-converting organic material that after continuous excitation features a broad low-energy emission band, resulting in high quality white light-emitting diodes. The benefits of this approach are the ease of fabrication and its low cost, since the current white LEDs are based on rare-earth-based phosphors as color converters which, in addition, cannot be combined with the encapsulation process, increasing the number of steps in the device fabrication. The most used down-converting materials are polymers, small-molecules, coordination complexes, and quantum dots. These materials are typically coated either onto the LED chip or onto glass substrates, which are placed on top of the LED. While the latter is considered as an excellent proof-of-concept without any possibility for commercial purposes, the former requires the preparation of the mixtures with UV-curable sealing reagents—silicones—that need to be directly applied onto the LED chip. This compromises the fabrication process in terms of chip damage, costs, the lack of a homogenous encapsulation in any kind of 3D forms, and the preparation of multilayered encapsulation systems featuring a cascade energy transfer process to fine tune the device chromaticity. Up to date, there are no examples of multilayered cascade encapsulating systems, but only one example, in which a fluorescent protein is entrapped into polystyrene microspheres, which are used as down-converting materials in LEDs (Hui KN et al., Nanotechnology. 2008, 19(35), 355203).

Thus, there are unfortunately several roadblocks to implement HLEDs as a daily used technology. Firstly, most of the examples have used blue-LEDs to provide the high-energy component of the white light. This results in a limitation to reach simultaneously high correlated color temperature (CCT) and color rendering index (CRI) at high brightness outputs (Heliotis G et al., Adv Mater. 2006, 18, 334; Gu E et al., Appl Phys Lett. 2007, 90, 031116; Huyal I O et al., J Mater Chem. 2008, 18, 3568; Kim O et al., ACS Nano 2010, 4, 3397; Stupca M et al., J Appl Phys. 2012, 112, 074313; Ban D et al., Phys Status Solidi Curr Top Solid State Phys. 2012, 9, 2594; Jang E-P et al., Nanotechnology 2013, 24, 045607; Findlay N J et al., J Mater Chem C 2013, 1, 2249; Lai C-F et al., Opt Lett. 2013, 38, 4082; Chen J et al., J Mater Sci. 2014, 49, 7391; Kim J-Y, Opt Commun. 2014, 321, 86; Shen P-C et al., Sci Rep. 2014, 4, 5307; Findlay NJ et al., Adv Mater. 2014, 26, 7290), pointing out to the use of UV-LEDs as a preferred choice to achieve a mature control of the color mixture. Secondly, the choice of the organic moiety seems to be the main bottleneck in terms of efficiency and stability. On one hand, the most explored down-converting materials, namely polymers, small-molecules, coordination complexes, and quantum dots show a limitation in combining a sharp absorption spectrum featuring high extinction coefficients with a highly-efficient broad emission band. In addition, there are a few intrinsically white emitters (Sun C-Y et al., Nat Commun. 2013, 4, 2717; Bao L et al., Curr Org Chem. 2014, 740). As such, white HLEDs typically feature an electroluminescence (EL) spectrum with two maxima peaks, one from the LED in the blue region and another from the organic part in the orange region, compromising the color quality in the red region of the visible spectrum. On the other hand, the application of the down-converting materials in an encapsulating system requires their mixture with UV- or thermal-curable sealing reagents. This procedure introduces problems like degradation of the organic down-converting material, as well as an uncontrolled phase separation (Huyal I O et al., J Mater Chem. 2008, 18, 3568), which, for example, hampers the necessary energy transfer process if two complementary emitters are blended together.

Enzyme-based detection kits are commonly used in diagnostics to determine concentrations of relevant biomarkers, metabolites and nucleic acids in a range of biological liquids including blood and urine. Based on rapid degradation of most enzymes under ambient temperatures, these kits are stored at −20° C. and transport requires a functional cold chain. Therefore, it would be advantageous to stabilize proteins for storage under room temperature. To enable routine use and to stabilize proteins for storage under ambient conditions, enzymes are immobilized or adsorbed to active surfaces and dehydrated. Most commonly, these procedures include cross-linking steps, which often negatively influence enzyme activity. Alternatively, enzymes are freeze-dried in the presence of stabilizing ingredients. The formulation of proteins has considerable impact on enzyme stability, activity and degradation during the freeze-drying process. The different physicochemical properties of enzymes make it almost impossible to design a universal formulation procedure to stabilize all enzymes. Furthermore, buffers used for formulation may include amino acids, sugars or sugar alcohols which might negatively influence the enzyme assay. Therefore, a universal method for immobilization of enzymes avoiding cross-linking and addition of stabilizing molecules would be needed.

US 2004/0156906 describes a thermosensitive and biodegradable microgel with a chemically crosslinked network comprising at least one negative temperature-sensitive macromolecule and one biodegradable group having a specific structure. This document relates exclusively to microgels containing crosslinked copolymers and fails to disclose any gel that would comprise, inter alia, both a specific branched polymer and a linear polymer, as it is the case for the gel provided in accordance with the present invention. Gill I et al., Trends Biotechnol. 2000, 18(11), 469-479 relates to the non-sol-gel encapsulation of proteins within different polymers, including certain biocomposites of proteins and crosslinked polyurethane polymers. US 2013/0313593 describes a specific light-emitting diode (LED) lighting apparatus. Weber MD et al., Adv Mater. 2015, 27(37), 5493-5498 and Niklaus L et al., Mater Horiz. 2016, doi:10.1039/C6MH00038J were published only after the priority date of this specification and refer to certain aspects of the present invention.

As described above, only a few approaches have been proposed to immobilize proteins in an elastomeric matrix and/or in a rubber-like material, which show excellent mechanical properties for different technological applications, while keeping the activity of the protein. In particular, the known fabrication procedures involve multicomponent mixtures as well as curable steps using UV and/or gamma irradiation, chemical reactions, and thermal treatments. These facts limit the number and/or nature of the proteins and, in turn, their technological application. Thus, there is a need in the art for improved rubber-like protein-based materials with respect to their preparation, universality, and their final technological applications that require a coating in 3D substrates and/or miniaturization to overcome the above-described drawbacks. For instance, there is a need of rubber-like materials as encapsulating systems for hybrid light-emitting diodes that can be used in a down-converting approach to develop cheap white light-emitting diodes. Specifically, there is a need of environmentally friendly, cheap and stable rubber-materials that can be deposited onto any kind of 3D substrates and/or encapsulation architectures like multilayers. In addition, an easy to upscale method for fabricating multilayered architectures is needed, which allow efficient cascade energy transfer schemes within the layers, solving the problem of phase separation between components, exciton quenching, etc.

The present invention addresses the above-discussed shortcomings and solves the problem of providing novel and improved means of immobilizing and stabilizing a wide range of different proteins, including luminescent proteins and enzymes, without requiring any crosslinking or curing. The invention also solves the problem of providing a novel and improved material containing luminescent or fluorescent proteins immobilized therein, which can be used as an environmentally friendly down-converting encapsulation material for hybrid light-emitting diodes. The invention further addresses the need for a novel and improved material containing enzymes or other proteins immobilized therein, which can advantageously be used in diagnosis or in bioreactors.

In particular, the present invention is based on the finding that, surprisingly, the addition of a protein in water or an aqueous solution to a mixture of a branched polymer such as trimethylolpropane ethoxylate and a linear polymer such as poly(ethylene oxide), followed by a partial dehydration/drying step, results in the formation of a rubber-like material in which the protein is immobilized/entrapped while its activity is retained. This has been demonstrated for various different luminescent proteins and also for a number of enzymes belonging to different EC classes, including a hydrolase (yeast invertase), a kinase (yeast hexokinase) and an isomerase (yeast phosphoglucoseisomase), which indicates a universal applicability of the immobilization technique of the present invention. The rubber-like material provided in accordance with the present invention is furthermore advantageous in that it can be prepared without any chemical cross-linking reactions, without any thermal and/or irradiation treatments, and without requiring any previous functionalization of the protein to be immobilized. It can easily be transferred onto any organic or inorganic support featuring any known 3D form, or can be prepared directly on such support. The application of the rubber-like material containing a luminescent protein immobilized therein for fabricating a cascade energy transfer encapsulation system for hybrid light-emitting diodes on any kind of 3D substrate without the use of conventional deposition techniques like drop-coating, spin-coating, spray-coating, doctor-blading, etc. has also been demonstrated.

Accordingly, in a first aspect the present invention provides a process of preparing a rubber-like material containing a protein immobilized therein, the process comprising the following steps: (a) mixing a protein, a branched polymer and a linear polymer in an aqueous solution to form a gel; and (b) drying the gel to obtain a rubber-like material containing the protein immobilized therein; wherein the branched polymer comprises at least three polymeric branches bound to a central branching unit.

In a second aspect, the invention also relates to a rubber-like material containing a protein immobilized therein, which is obtainable by the process according to the first aspect of the invention. Moreover, in this second aspect, the invention provides a rubber-like material containing a protein immobilized therein, wherein the rubber-like material comprises a branched polymer and a linear polymer, and wherein the branched polymer comprises at least three polymeric branches bound to a central branching unit.

The invention furthermore relates to the preparation of the gel that is obtained in step (a) of the above-described process according to the first aspect of the invention. Thus, in a third aspect, the invention provides a process of preparing a gel, the process comprising: (a) mixing a protein, a branched polymer and a linear polymer in an aqueous solution to form a gel; wherein the branched polymer comprises at least three polymeric branches bound to a central branching unit.

In a fourth aspect, the invention relates to a gel which is obtainable by the process according to the above-described third aspect of the invention. In this fourth aspect, the invention also provides a gel comprising a protein, a branched polymer and a linear polymer, wherein the branched polymer comprises at least three polymeric branches bound to a central branching unit.

In a fifth aspect, the present invention relates to the use of the rubber-like material according to the second aspect in lighting, particularly as an environmentally friendly down-converting material for a hybrid light-emitting diode (e.g., a hybrid white light-emitting diode). In accordance with this fifth aspect, the invention also relates to the use of the rubber-like material of the second aspect as a down-converting cascade energy transfer encapsulation for a hybrid light-emitting diode (e.g., a hybrid white light-emitting diode). Moreover, in this fifth aspect the invention further provides a hybrid light-emitting diode (e.g., a hybrid white light-emitting diode) comprising a light-emitting diode and a coating, wherein the coating contains one or more (e.g., one, two, three, four, or five) layers of the rubber-like material according to the second aspect of the invention. In the fifth aspect of the invention, the protein immobilized in the rubber-like material is a luminescent protein, preferably a fluorescent protein.

In a sixth aspect, the invention relates to the in vitro use of the rubber-like material according to the second aspect in diagnosis, e.g., for the detection of one or more metabolites or nucleic acids in a sample such as blood, plasma, urine, or any other body liquid. The invention also relates to the use of the rubber-like material of the second aspect in a diagnostic device or kit, and to a diagnostic device or kit comprising the rubber-like material according to the second aspect.

In a seventh aspect, the present invention provides the use of the rubber-like material according to the second aspect in a bioreactor. The invention also provides a bioreactor comprising the rubber-like material according to the second aspect. In this seventh aspect of the invention, the protein immobilized in the rubber-like material is an enzyme.

The following description of general and preferred features and embodiments relates to each one of the processes, products and uses provided in the present specification, including in particular those according to the above-described first, second, third, fourth, fifth, sixth and seventh aspects of the invention, unless explicitly indicated otherwise.

The branched polymer to be used in accordance with the present invention comprises at least three polymeric branches bound to a central branching unit. Accordingly, in the branched polymer there are three or more polymeric branches which are each bound to a single moiety of the branched polymer, which moiety is referred to as “central branching unit”.

The central branching unit comprised in the branched polymer is preferably a C₁₋₂₀ hydrocarbon moiety which is substituted with 3 to 8 substituent groups, wherein said substituent groups are each independently selected from hydroxy, carboxy and amino, and further wherein each of the at least three polymeric branches of the branched polymer is bound to one of the substituent groups of the C₁₋₂₀ hydrocarbon moiety; optionally, one or more carbon atoms (e.g., one, two, three, four or five carbon atoms) comprised in the C₁₋₂₀ hydrocarbon moiety are each independently replaced by an oxygen atom, a nitrogen atom or a sulfur atom, preferably by an oxygen atom. It is to be understood that each one of the at least three polymeric branches is bound to a different substituent group on the above-mentioned hydrocarbon moiety, and that the maximum number of polymeric branches that can be bound to such a central branching unit is identical to the maximum number of substituent groups on the hydrocarbon moiety. The above-mentioned hydrocarbon moiety is preferably a C₃₋₂₀ hydrocarbon moiety, more preferably a C₃₋₁₅ hydrocarbon moiety, and even more preferably a C₄₋₁₀ hydrocarbon moiety. The hydrocarbon moiety is preferably substituted with 3, 4, 5 or 6 substituent groups, more preferably with 3, 4 or 5 substituent groups, even more preferably with 3 or 4 substituent groups, and yet even more preferably with 3 substituent groups. The substituent groups on the hydrocarbon moiety are each independently selected from hydroxy (—OH), carboxy (—COOH) and amino (—NH₂), and are preferably each hydroxy. If a substituent group which is attached to a polymeric branch is a hydroxy group, then it is preferred that the polymeric branch is attached to said hydroxy group via an ether linkage or via an ester linkage, more preferably via an ether linkage (as, e.g., in the exemplary branched polymer trimethylolpropane ethoxylate). If a substituent group which is attached to a polymeric branch is a carboxy group, it is preferred that the polymeric branch is attached to said carboxy group via an ester linkage or via an amide linkage. If a substituent group which is attached to a polymeric branch is an amino group, it is preferred that the polymeric branch is attached to said amino group via an amide linkage.

Accordingly, it is preferred that the central branching unit is a C₃₋₂₀ hydrocarbon moiety which is substituted with 3 to 8 substituent groups, wherein said substituent groups are each independently selected from hydroxy, carboxy and amino, optionally wherein one or more carbon atoms (e.g., one, two, three, four or five carbon atoms) comprised in said C₃₋₂₀ hydrocarbon moiety are each replaced by an oxygen atom, and further wherein each of the at least three polymeric branches is bound to one of the substituent groups of the C₃₋₂₀ hydrocarbon moiety. More preferably, the central branching unit is a C₃₋₂₀ hydrocarbon moiety which is substituted with 3 to 8 hydroxy groups, optionally wherein one or more carbon atoms (e.g., one, two, three, four or five carbon atoms) comprised in said C₃₋₂₀ hydrocarbon moiety are each replaced by an oxygen atom, and further wherein each of the at least three polymeric branches is bound to one of the hydroxy groups of the C₃₋₂₀ hydrocarbon moiety. Even more preferably, the central branching unit is selected from a trimethylolpropane moiety, a trimethylolethane moiety, a trimethylolmethane moiety, a glycerol moiety, a pentaerythritol moiety, a pentaerythrithiol moiety, a diglycerol moiety, a triglycerol moiety, a dipentaerythritol moiety, a tetraglycerol moiety, a pentaglycerol moiety, a tripentaerythritol moiety, a hexaglycerol moiety, a trimethanolamine moiety, a triethanolamine moiety, a triisopropanolamine moiety, a propane-1,2,3-tricarboxylic acid moiety, a citric acid moiety, an isocitric acid moiety, a trimesic acid moiety, a 1,1,1-tris(aminomethyl)propane moiety, a 1,1,1-tris(aminomethyl)ethane moiety, a tris(aminomethyl)methane moiety, a propane-1,2,3-triamine moiety, a tris(2-aminoethyl)amine moiety, and a tris(carboxymethyl)ethylenediamine moiety. The central branching unit may also be a tri(C₁₋₈ alkanol)amine moiety or a tri(hydroxy-C₁₋₈ alkylene)amine moiety. Still more preferably, the central branching unit is selected from a trimethylolpropane moiety, a trimethylolethane moiety, a trimethylolmethane moiety, and a glycerol moiety. Most preferably, the central branching unit is a trimethylolpropane moiety, as shown in the following:

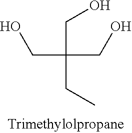

The at least three polymeric branches comprised in the branched polymer can all be the same or can be different from one another, and are preferably the same. It is preferred that the branched polymer has 3 to 8 polymeric branches, more preferably 3, 4, 5 or 6 polymeric branches, even more preferably 3, 4 or 5 polymeric branches, yet even more preferably 3 or 4 polymeric branches, and most preferably 3 polymeric branches. The polymeric branches may be linear or branched, and may also be dendritically branched. Preferably, each of the polymeric branches is a linear polymer. More preferably, the polymeric branches are each independently a poly(alkylene oxide) having a terminal —OH, —OR, —O—CO—R, —CO—O—R, or —CO—N(R)—R group, wherein each R is independently C₁₋₅ alkyl (e.g., methyl or ethyl) or C₂₋₅ alkenyl (e.g., vinyl). The poly(alkylene oxide) may be, e.g., a poly(ethylene oxide), a poly(propylene oxide), or a copolymer of ethylene oxide and propylene oxide (e.g., a block copolymer of ethylene oxide (EO) and propylene oxide (PO), such as a block copolymer having the structure EO—PO-EO), and is preferably a poly(ethylene oxide). Moreover, the poly(alkylene oxide) preferably has a terminal —OH, —OR or —O—CO—R group, more preferably a terminal —OH group. The aforementioned terminal group is present at a different or opposite end of the poly(alkylene oxide) in relation to the point of attachment of the poly(alkylene oxide) to the central branching unit. The polymeric branches comprised in the branched polymer are even more preferably each independently a poly(ethylene oxide), a poly(propylene oxide), or a copolymer of ethylene oxide and propylene oxide, wherein said poly(ethylene oxide), said poly(propylene oxide) and said copolymer each have a terminal —OH, —OR or —O—CO—R group, wherein each R is independently C₁₋₅ alkyl. Yet even more preferably, the polymeric branches are each independently a poly(ethylene oxide) having a terminal -OH group.

It is preferred that the branched polymer has a number average molecular weight of about 200 Da to about 2000 Da, more preferably of about 300 Da to about 1200 Da. It is furthermore preferred that the branched polymer is water-soluble at a temperature of 25° C. and an absolute pressure of 1 bar (100 kPa).

It is particularly preferred that the branched polymer is trimethylolpropane ethoxylate (TMPE), the structure of which is illustrated in the following (wherein each variable n denotes the number of repeating ethylene oxide monomer units of the corresponding polymeric branch):

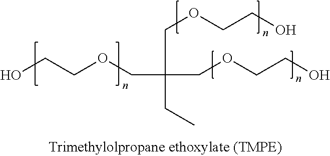

Even more preferably, the branched polymer is trimethylolpropane ethoxylate having a number average molecular weight of about 300 Da to about 1200 Da (e.g., about 450 Da, about 730 Da, or about 1040 Da), yet even more preferably the branched polymer is trimethylolpropane ethoxylate having a number average molecular weight of about 350 Da to about 550 Da, and most preferably the branched polymer is trimethylolpropane ethoxylate having a number average molecular weight of about 450 Da.

A further example of the branched polymer is trimethylolpropane ethoxylate methyl ether diacrylate (TMPEMED), such as TMPEMED having a number average molecular weight of about 70 Da to about 2000 Da, preferably of about 200 Da to about 800 Da, more preferably of about 388 Da:

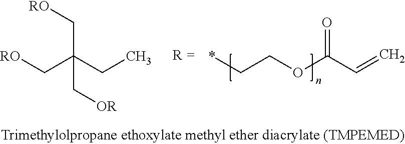

A further example of the branched polymer is polyethylenimine (PEI), such as PEI having a weight average molecular weight of about 200 Da to about 3000 Da, preferably of about 400 Da to about 1600 Da, more preferably of about 800 Da:

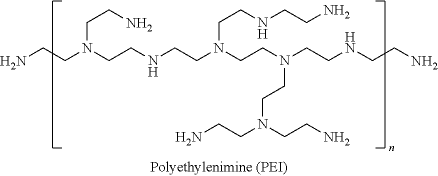

It is possible to use a single branched polymer, i.e. a single type of branched polymer, or to use two or more (e.g., two, three, four, or five) different branched polymers.

The linear polymer to be used in accordance with the present invention is not particularly limited. It preferably has a number average molecular weight of about 10 kDa to about 10,000 kDa, more preferably of about 500 kDa to about 7000 kDa. It is furthermore preferred that the linear polymer is water-soluble at a temperature of 25° C. and an absolute pressure of 1 bar (100 kPa). The linear polymer may be, for example, a poly(alkylene oxide) having a terminal group at each of its two ends which is selected independently from —OH, —OR, —O—CO—R, —CO—O—R and —CO—N(R)—R, wherein each R is independently C₁₋₅ alkyl (e.g., methyl or ethyl) or C₂₋₅ alkenyl (e.g., vinyl), or the linear polymer may be a poly(acrylic acid) or a poly(4-styrenesulfonic acid). The poly(alkylene oxide) may be, e.g., a poly(ethylene oxide), a poly(propylene oxide), or a copolymer of ethylene oxide and propylene oxide (e.g., a block copolymer of ethylene oxide (EO) and propylene oxide (PO), such as a block copolymer having the structure EO—PO-EO), and is preferably a poly(ethylene oxide). Moreover, the terminal group at each of the two ends of the poly(alkylene oxide) is preferably selected independently from —OH, —OR and —O—CO—R, and is more preferably an —OH group.

Accordingly, it is particularly preferred that the linear polymer is a poly(alkylene oxide) having a terminal group at each of its two ends which is selected independently from —OH, —OR and —O—CO—R, wherein each R is independently C₁₋₅ alkyl. More preferably, the linear polymer is a poly(ethylene oxide), a poly(propylene oxide) or a copolymer of ethylene oxide and propylene oxide, wherein said poly(ethylene oxide), said poly(propylene oxide) or said copolymer has a terminal group at each of its two ends, which terminal group is selected independently from —OH, —OR and —O—CO—R, wherein each R is independently C₁₋₅ alkyl. Even more preferably, the linear polymer is a poly(ethylene oxide) having a terminal —OH group at each of its two ends. Yet even more preferably, the linear polymer is a poly(ethylene oxide) with a terminal —OH group at each of its two ends, having a number average molecular weight of about 2000 kDa to about 7000 kDa, still more preferably of about 4000 kDa to about 6000 kDa. Most preferably, the linear polymer is a poly(ethylene oxide) with a terminal —OH group at each of its two ends, having a number average molecular weight of about 5000 kDa.

A further example of the linear polymer is poly(2-ethyl-2-oxazoline) (PEOx), such as PEOx having a number average molecular weight of about 100 kDa to about 2500 kDa, preferably of about 250 kDa to about 1000 kDa, more preferably of about 500 kDa:

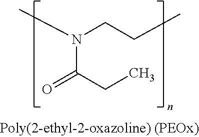

It is possible to use a single linear polymer, i.e. a single type of linear polymer, or to use two or more (e.g., two, three, four, or five) different linear polymers.

The protein to be used in accordance with the invention is not particularly limited, but is preferably a luminescent protein or an enzyme. The luminescent protein is preferably a fluorescent protein, such as, e.g., green fluorescent protein (GFP), enhanced green fluorescent protein, blue fluorescent protein, cyan fluorescent protein, teal fluorescent protein, yellow fluorescent protein, orange fluorescent protein, red fluorescent protein, near-infrared fluorescent protein, mCherry, mStrawberry, mRaspberry, mOrange, mCitrine, tdTomato, mTagBFP, dsRed, UnaG, eqFP611, Dronpa, TagRFPs, KFP, EosFP, Dendra, or IrisFP. The enzyme is preferably an oxidoreductase, a transferase, a DNA polymerase, an RNA polymerase, a kinase, a hydrolase, a lyase, an isomerase, or a ligase. The protein may also be a fusion protein comprising a luminescent protein or an enzyme (e.g., any of the above-mentioned specific luminescent proteins or enzymes), which is fused, optionally via a linker (e.g., a glycine and/or serine rich linker, such as a linker composed of 2 to 35 amino acids, preferably 5 to 10 amino acids, selected independently from glycine and serine, or any one of the linkers mentioned in Reddy Chichili V P et al., Protein Sci. 2013, 22(2):153-67, including in Table 1 of this reference), to an adaptor protein domain (e.g., an Src homology 2 domain (SH2 domain), an Src homology 3 domain (SH3 domain), or a poly(A)-binding protein C-terminal domain (PABC domain)). The polymer is preferably water-soluble at a temperature of 25° C. and an absolute pressure of 1 bar (100 kPa). It is possible to use a single protein, i.e. a single type of protein, or to use two or more (e.g., two, three, four, or five) different proteins, e.g., two or more different luminescent proteins or two or more different enzymes.

The branched polymer, the linear polymer and the protein can be prepared using methods known in the fields of synthetic chemistry or molecular biology, and/or are commercially available.

The aqueous solution to be used in step (a) of the process according to the first or the third aspect of the invention is not particularly limited, and is preferably water or an aqueous buffer solution. Examples of suitable aqueous buffer solutions include, in particular, phosphate buffer,

HEPES buffer, Tris buffer, MOPS buffer, MES buffer, TES buffer, CHES buffer, PIPES buffer, CAPS buffer, HEPPS buffer, imidazole buffer, tricine buffer, bicine buffer, glycine buffer, citric acid buffer, or acetic acid buffer. The pH of the buffer can be adjusted using, e.g., HCl or NaOH (or KOH) as desired for the protein to be immobilized, particularly to a pH at which the protein remains correctly folded (e.g., about pH 6, about pH 7, or about pH 8), or to the optimum pH of enzyme activity if the protein is an enzyme. A preferred exemplary aqueous buffer solution is phosphate-buffered saline (PBS), which can be prepared, e.g., according to the Cold Spring Harbor Protocol (doi:10.1101/pdb.rec8247). It is particularly preferred that the protein is provided in an aqueous buffer solution containing 50 mM NaH₂PO₄ pH 8.0, 300 mM NaCl, and 250 mM imidazole (e.g., at a protein concentration of about 1 mg/mL to about 20 mg/mL). The aqueous buffer solution may further contain one or more protein stabilizers, such as poly(ethyleneimine) (PEI), ethylenediaminetetraacetic acid (EDTA), ammonium sulfate, trehalose, or a commercially available protein stabilizer such as “Thermo Scientific Protein Stabilizing Cocktail” (Life Technologies, product no. 89806). Any other aqueous solvent or aqueous medium (e.g., containing at least about 60 vol-% water, preferably at least about 70 vol-% water, more preferably at least about 80 vol-% water, even more preferably at least about 90 vol-% water, still more preferably at least about 95 vol-% water) can also be used in place of the above-described aqueous solution in order to ensure the stability of the protein.

The amounts or mass ratios between the branched polymer, the linear polymer and the protein employed in step (a) of the process according to the first or the third aspect of the invention can be adjusted to obtain a gel having enough viscosity for coating and/or printing purposes.

In particular, it is preferred that in step (a) of the process according to the first or the third aspect of the invention, the branched polymer and the linear polymer are mixed in a mass ratio of 3:1 to 20:1 (branched polymer : linear polymer), more preferably in a mass ratio of 4:1 to 15:1, and even more preferably in a mass ratio of 6:1 to 12:1.

If the branched polymer and the linear polymer are mixed in step (a) in a mass ratio of about 12:1 (branched polymer : linear polymer), it is particularly preferred that the total volume of the aqueous solution employed in step (a) is about 15 μl to about 50 μl per mg of linear polymer, more preferably about 20 μl to about 40 μl per mg of linear polymer.

The amount of the protein to be employed in step (a) of the process according to the first or the third aspect of the invention is not particularly limited. For example, the protein can be employed in step (a) in an amount of about 3 mass-% to about 35 mass-% with respect to the mass of the linear polymer, which is particularly preferred if the protein is a luminescent protein (such as, e.g., a fluorescent protein). It is even more preferred that the protein is employed in step (a) in an amount of about 8 mass-% to about 15 mass-%, and yet even more preferably in an amount of about 10 mass-%, with respect to the mass of the linear polymer if the protein is a luminescent protein (e.g., a fluorescent protein). If the protein is an enzyme, smaller amounts can be used. For example, if the protein is an enzyme, it can be employed in step (a) of the process according to the first or the third aspect of the invention in an amount of about 1 mass-ppm to about 1 mass-% with respect to the mass of the linear polymer, preferably in an amount of about 0.001 mass-% to about 0.5 mass-% with respect to the mass of the linear polymer.

In the first and the third aspect of the invention, it is preferred that step (a) comprises first mixing the branched polymer and the linear polymer, subsequently adding the protein in an aqueous solution and mixing the protein, the branched polymer and the linear polymer in the aqueous solution, and optionally adding further aqueous solution during said mixing, to form a gel. Alternatively, step (a) can also be conducted by providing the protein in an aqueous solution, adding the branched polymer and the linear polymer to the aqueous solution of the protein, mixing the protein, the branched polymer and the linear polymer in the aqueous solution, and optionally adding further aqueous solution during said mixing, to form a gel. In either case, it is preferred that said mixing of the protein, the branched polymer and the linear polymer in step (a) is conducted under stirring (e.g., at 1500 rpm). It is furthermore preferred that step (a) of the process according to the first or the third aspect is conducted under ambient conditions, particularly at a temperature of about 15° C. to about 35° C., preferably at about 20° C. to about 30° C., and more preferably at about 25° C.

The processes according to the present invention are particularly advantageous in that they allow the preparation of a rubber-like material containing a protein immobilized therein (or a corresponding gel which can be dried to obtain the rubber-like material) without the need for any crosslinking or curing of the polymers that are used. It is thus preferred that the process of the first or the third aspect of the invention does not comprise any step of thermally curing, UV-curing or crosslinking the polymers that are mixed in step (a). It is likewise preferred that the process does not comprise any step of covalently crosslinking the polymers that are mixed in step (a). Furthermore, in accordance with each one of the various aspects of the present invention, it is preferred that the branched polymer and the linear polymer are not covalently crosslinked. It is also preferred that the branched polymer and the linear polymer (as present, e.g., in the rubber-like material according to the second aspect or in the gel according to the fourth aspect of the invention) are free of covalent crosslinkages.

In the process according to the first aspect of the invention, step (b) comprises drying the gel obtained in step (a) in order to obtain the rubber-like material containing the protein immobilized therein. Preferably, in step (b) the gel is partially dehydrated to obtain the rubber-like material containing the protein immobilized therein. For example, the gel may be partially dehydrated via vacuum drying, freeze-drying, drum-drying, spray drying, or sunlight-ambient evaporation. It is particularly preferred that the gel is partially dehydrated using a vacuum. Accordingly, it is preferred that in step (b) the gel is partially dehydrated in a vacuum station/chamber (e.g., at a pressure of about 1 mbar to about 10 mbar for a period of less than or equal to about 1 hour, or alternatively at a pressure of about 10⁻⁵ bar to about 10⁻⁹ bar for a period of about 5 seconds to about 5 minutes). Before the gel is introduced into the vacuum station/chamber, it can be deposited onto a substrate using any coating or printing method, particularly a solvent-based technique. Exemplary techniques for depositing the gel onto a substrate include, in particular, doctor-blading, roll-to-roll coating, spin coating, gravure printing, inkjet printing, flexographic printing, screen printing, or 3D printing. The gel can thereby be deposited onto any suitable substrate, including any of the specific substrates mentioned further below.

The rubber-like material can be prepared, e.g., in the form of a film having a thickness of about 10 nm to about 10 mm, preferably of about 10 μm to about 10 mm.

The rubber-like material according to the second aspect of the invention or the rubber-like material prepared in accordance with the first aspect of the invention can further be deposited onto a substrate/support. For example, the rubber-like material can be mechanically deposited onto a substrate, e.g., using tweezers. The present invention specifically relates to the rubber-like material deposited on a substrate as well as a corresponding process of preparation, the process comprising depositing the rubber-like material onto a substrate. Alternatively, the gel according to the fourth aspect of the invention can be dried on a substrate (e.g., as described above in connection with step (b) of the process according to the first aspect of the invention) in order to directly obtain the rubber-like material deposited on the corresponding substrate. As a further alternative, the rubber-like material deposited on a substrate can also be prepared using a process comprising: (a) introducing a substrate into the gel according to the fourth aspect of the invention (or into the gel obtained in the process according to the third aspect of the invention); and (b) drying the gel on the substrate to obtain a rubber-like material deposited on the substrate. This latter process is particularly suitable for depositing the rubber-like material onto a three-dimensional substrate. In step (b) of this latter process, the gel may be partially dehydrated via vacuum drying, freeze-drying, drum-drying, spray drying, or sunlight-ambient evaporation, and is preferably partially dehydrated using a vacuum. Each of the various procedures described in this paragraph can be repeated until the desired thickness of the layer of rubber-like material on the substrate/support is reached (e.g., between about 20 μm and about 10 mm).

The substrate/support onto which the rubber-like material can be deposited is not particularly limited, and may be selected from organic materials such as carboxymethyl-cellulose, starch, collagen, modified sepharose, ion exchange resins, active charcoal, polymers, active membranes, etc. or from inorganic materials such as silica, clay, metal oxides, diatomaceous earth, hydroxyapatite, ceramic, glass, etc. Flexible substrates may be also used, e.g., substrates based on polymeric materials, such as poly(ethylene terephthalate), poly(ethylene naphthalate), poly(imide), poly(carbonate), or combinations or derivatives thereof. The substrate may also comprise or consist of paper or a paper-like material. Preferably, the substrate onto which the rubber-like material can be deposited is selected from carboxymethyl-cellulose, starch, collagen, silica, clay, metal oxide, diatomaceous earth, hydroxyapatite, ceramic, glass, paper, poly(ethylene terephthalate), poly(ethylene naphthalate), poly(imide), poly(carbonate), and combinations thereof. In particular, the substrate may be a three-dimensional substrate made of any of the aforementioned materials.

The rubber-like material according to the present invention protects the protein immobilized therein from degradation or damage (e.g., caused by ambient conditions such as room temperature, room light and/or humid air, by elevated temperatures, and/or by exposure to sunlight), which provides considerable advantages in terms of storage and transportation, and furthermore allows to omit the use of costly cooling systems which may otherwise be necessary. This also makes the rubber-like material particularly advantageous for diagnostic applications, in which cooling is often a critical factor. Thus, in accordance with the sixth aspect, the present invention relates to the in vitro use of the rubber-like material in diagnosis, wherein the rubber-like material has not been cooled prior to its use in diagnosis. The invention also relates to the in vitro use of the rubber-like material in diagnosis, wherein prior to its use in diagnosis the rubber-like material has been stored without cooling and/or has been stored at a temperature of about 20° C. to about 35° C. It is likewise preferred that the diagnostic device or kit according to the sixth aspect of the invention does not comprise any cooling system. The diagnostic device or kit may be a single-use diagnostic device or kit. Moreover, the diagnostic device or kit may comprise or consist of the rubber-like material (containing a protein immobilized therein) deposited on a substrate (e.g., on any of the specific substrates described herein above). The protein immobilized in the rubber-like material to be used in accordance with the sixth aspect is preferably an enzyme (e.g., any of the specific enzymes described herein above).

The hybrid light-emitting diode (hybrid LED) according to the fifth aspect of the invention may, e.g., comprise or consist of a bottom inorganic LED, optionally a first encapsulation on the inorganic LED, and a second encapsulation composed of the rubber-like material comprising a luminescent protein immobilized therein. This second encapsulation is preferably multilayered, and may be placed directly on top of the inorganic LED or on top of the first encapsulation (which may be made of an organic and/or an inorganic material, and may have any 3D form). The luminescent protein to be used in the fifth aspect may be any one of the specific luminescent or fluorescent proteins described herein above. Upon excitation by the inorganic LED, such luminescent proteins partially convert the high-energy photons into low-energy photons, which sum up to the non-absorbed high-energy photons from the inorganic LED, resulting in a color change of the inorganic LED.

In accordance with the fifth aspect, the invention relates, in particular, to the use of the rubber-like material containing a luminescent protein immobilized therein as a down-converting cascade energy transfer encapsulation for a hybrid LED. Such a cascade energy transfer encapsulation typically comprises or consists of several layers (e.g., two, three, four, five, six, seven, eight, or more layers) of the rubber-like material containing a luminescent protein immobilized therein, wherein the luminescent proteins in each pair of neighboring layers have complementary absorption and emission features. The bottom layers may, e.g., emit high-energy photons upon excitation from the inorganic LED that are partially converted by the top layer into low-energy photons. The combination of the non-absorbed high-energy photons from the inorganic LED, the bottom down-converting layer, and the top down-converting layer preferably results in a white LED. The rubber-like material containing a luminescent protein immobilized therein can thus be used for fabricating white light-emitting diodes using an innovative cascade energy transfer encapsulating system, which circumvents the problems related to phase separation and exciton-loss in multicomponent single-layer down-converting encapsulation systems. The encapsulation system according to the fifth aspect of the invention features a higher rendering color with advantageous stabilities under high luminance inputs and advantageous luminous efficiencies when the device is running under ambient conditions. Furthermore, the light output of the device can be easily modified by the thickness of the rubber-like material, thus covering the whole visible spectrum.

In accordance with the present invention, it is also envisaged to immobilize/entrap molecules or materials other than proteins in the rubber-like material described herein. In particular, in each one of the various processes, products and uses described in this specification, including those according to the first, second, third, fourth, fifth, sixth and seventh aspect of the invention, it is also possible to use an active material (particularly a non-protein active material) instead of the respective protein, and the present invention also relates to this possibility. The active material (or non-protein active material) is not particularly limited and may be selected, e.g., from small molecules, coordination complexes, polymers, quantum dots (including, in particular, carbon quantum dots or carbon-based quantum dots), and nanoparticles (including, in particular, luminescent nanoparticles). Porphyrins, perylenediimide (PDI) or derivatives thereof, cumarins, neutral/charged coordination complexes like [Ru(bpy)₃][PF₆], [Ir(ppy)₂(bpy)][PF₆] or [Ir(ppy)₂(acac)] (preferably [Ru(bpy)₃][PF₆] or [Ir(ppy)₂(acac)]), luminescent polymers like poly[(9,9-di-n-octylfluorenyl-2,7-diyl)-alt-(benzo[2,1,3]thiadiazol-4,8-diyl)] (F8BT), polyfluorenes (PFO), poly(1,4-phenylene) (PPP), poly(1,4-phenylene vinylene) (PPV), or nanoparticles like silica or ZnO can also be used. In particular, homoleptic or heteroleptic, neutral or charged coordination complexes based on a metal core of, e.g., Pt(II), Pd(I), Au(I), Ru(II), Ir(III), Os(II), Mn(II), Zn(II), Mg(II), Cu(I), or Al(III) can be used as the active material, with homoleptic or heteroleptic, neutral or charged coordination complexes based on a metal core of Pt(II), Pd(I), Au(I), Ru(II), Ir(III), Os(II), Mn(II), Cu(I), or Al(III) being preferred. Likewise, the active material may be a light-emitting polymer selected from 2,5-bis(chloromethyl)-1-methoxy-4-(2-ethylhexyloxy)benzene, MDMO-PPV, MEH-PPV (e.g., having an Mₙ of 40 kDa to 70 kDa, or an Mₙ of 70 kDa to 100 kDa, or an Mₙ of 150 kDa to 250 kDa), methyl viologen dichloride hydrate, poly[2,5-bis(3′,7′-dimethyloctyloxy)-1,4-phenylenevinylene], poly[9,9-bis-(2-ethylhexyl)-9H-fluorene-2,7-diyl], poly[2-(2′,5′-bis(2″-ethylhexyloxy)phenyl)-1,4-phenylenevinylene], poly{[2[2′,5′-bis(2″-ethylhexyloxy)phenyl]-1,4-phenylenevinylene]-co-[2-methoxy-5-(2′-ethylhexyloxy)-1,4-phenylenevinylene]}, poly[2,5-bisoctyloxy)-1,4-phenylenevinylene], poly(2,5-bis(1,4,7,10-tetraoxaundecyl)-1,4-phenylenevinylene), poly(3-cyclohexylthiophene-2,5-diyl), poly(9,9-di-n-dodecylfluorenyl-2,7-diyl), poly[(9,9-dihexylfluoren-2,7-diyl)-co-(anthracen-9,10-diyl)], poly[(9,9-dihexylfluoren-2,7-diyl)-alt-(2,5-dimethyl-1,4-phenylene)], poly[(9,9-dihexylfluoren-2,7-diyl)-co-(9-ethylcarbazol-2,7-diyl)], poly(9,9-n-dihexyl-2,7-fluorene-alt-9-phenyl-3,6-carbazole), poly(9,9-di-n-hexylfluorenyl-2,7-diyl), poly(2,5-dihexyloxy-1,4-phenylenevinylene), poly(9,9-di-n-octylfluorenyl-2,7-diyl), poly(2,5-dioctylphenylene-1,4-ethynylene), poly(2,5-dioctyl-1,4-phenylenevinylene), poly[5-methoxy-2-(3-sulfopropoxy)-1,4-phenylenevinylene], poly(3-octylthiophene-2,5-diyl) regiorandom, poly[(m-phenylenevinylene)-alt-(2,5-dihexyloxy-p-phenylenevinylene)], poly[(o-phenylenevinylene)-alt-(2-methoxy-5-(2-ethylhexyloxy)-p-phenylenevinylene)], poly[(m-phenylenevinylene)-alt-(2-methoxy-5-(2-ethylhexyloxy)-p-phenylenevinylene)], poly[(p-phenylenevinylene)-alt-(2-methoxy-5-(2-ethylhexyloxy)-p-phenylenevinylene)], poly[tris(2,5-bis(hexyloxy)-1,4-phenylenevinylene)-alt-(1,3-phenylenevinylene)], poly(9-vinylcarbazole) (having a weight-average molecular weight of about 1100 kDa), and poly(p-xylene tetrahydrothiophenium chloride), or the active material may be any of the light-emitting polymers referred to in Pei Q, Material Matters, 2007, 2.3, 26. The active material may also be a non-protein dye, particularly a fluorescent dye or a phosphorescent dye. Exemplary dyes, particularly non-protein fluorescent dyes, that can be used as the active material include, without limitation, 7-aminoactinomycin D, 8-anilinonaphthalene-1-sulfonic acid, an Alexa Fluor dye, an ATTO dye, benzanthrone, bimane, 9,10-bis(phenylethynyl)anthracene, 5,12-bis(phenylethynyl)naphthacene, bisbenzimide, calcein, carboxyfluorescein, carboxyfluorescein diacetate succinimidyl ester, carboxyfluorescein succinimidyl ester, 1-chloro-9,10-bis(phenylethynyl)anthracene, 2-chloro-9,10-bis(phenylethynyl)anthracene, 2-chloro-9,10-diphenylanthracene, coumarin, 4′,6-diamidino-2-phenylindole (DAPI), 3,3′-dihexyloxacarbocyanine iodide (DiOC₆), a DyLight Fluor dye, epicocconone, FlAsH-EDT2, Fluo-3, Fluo-4, a FluoProbes dye, Fura-2, Fura-2-acetoxymethyl ester, a heptamethine dye (e.g., IR-780 or IR-808), iminocoumarin, Indian yellow, Indo-1, laurdan, Lucifer yellow, a merocyanine (e.g., merocyanine 540), Nile red, a perylene, phloxine B, a phycobilin (e.g., phycoerythrobilin, phycourobilin, phycoviolobilin, or phycocyanobilin), pyranine, a rhodamine (e.g., rhodamine B, rhodamine 123, or rhodamine 6G), RiboGreen, rubrene, (E)-stilbene, (Z)-stilbene, sulforhodamine 101, sulforhodamine B, SYBR Green I, SYBR Safe, tetraphenyl butadiene, tetrasodium tris(bathophenanthroline disulfonate)ruthenium(II), Texas Red, titan yellow, 6-methoxy-(8-p-toluenesulfonamido)quinoline (TSQ), umbelliferone, violanthrone, or YOYO-1. Preferably, a non-protein fluorescent dye to be used as the above-mentioned active material is selected from xanthene compounds (e.g., fluorescein, rhodamine, Oregon green, eosin, or Texas red), cyanine compounds (e.g., cyanine, indocarbocyanine, oxacarbocyanine, thiacarbocyanine, or merocyanine), squaraine compounds (e.g., Seta or SeTau), naphthalene compounds (e.g., dansyl or prodan compounds), coumarin compounds, oxadiazole compounds (e.g., pyridyloxazole, nitrobenzoxadiazole or benzoxadiazole), anthracene compounds (e.g., an anthraquinone, such as DRAQ5, DRAQ7 or CyTRAK Orange), pyrene compounds (e.g., cascade blue), oxazine compounds (e.g., Nile red, Nile blue, cresyl violet, or oxazine 170), acridine compounds (e.g., proflavin, acridine orange, or acridine yellow), arylmethine compounds (e.g., auramine, crystal violet, or malachite green), and tetrapyrrole compounds (e.g., porphin, phthalocyanine, or bilirubin). The rubber-like material containing a dye (including any of the aforementioned dyes) immobilized therein can be used, in particular, for the lighting applications according to the fifth aspect of the invention. The active material can be employed in any suitable solvent in which it can be dissolved, e.g., in an aqueous solution (such as water) or in acetonitrile (see Example 2, and particularly Table 3 in which the suitability of various different solvents has been assessed). Thus, if the protein to be used in accordance with the first, second, third, fourth, fifth, sixth or seventh aspect of the invention is replaced by an active material (as described above), not only an aqueous solution may be used, but also other solvents (e.g., acetonitrile) can be used in place of the aqueous solution in order to dissolve the active material, the branched polymer and the linear polymer. It is also possible to dissolve the active material in the branched polymer and then add water and the linear polymer and mix the active material, the branched polymer and the linear polymer, which is a preferred approach for any active material that is soluble in the branched polymer.

The following definitions apply throughout the present specification, unless specifically indicated otherwise.

As used herein, the term “polymer” refers to a molecule comprising two or more (e.g., five or more, preferably ten or more) repeating units of the same structure (also referred to as monomer units or repeating monomer units).

The term “linear polymer” refers to a polymer in which the monomer units are connected in a linear fashion, i.e., in the form of a single straight chain. A linear polymer does not have any covalent bonds or covalently bonded groups, which would connect its monomer units in a branched fashion or in a crosslinked fashion.

The term “branched polymer” refers to a polymer comprising at least one branching point (or branching moiety), through which three or more monomer units are covalently connected.

Accordingly, the branches of a “branched polymer” are composed of complete monomer units, whereas a linear polymer that contains side chains within individual monomer units (such as polystyrene) does not constitute a branched polymer.

The term “hydrocarbon moiety” or “hydrocarbon group” refers to a moiety or group consisting of carbon atoms and hydrogen atoms. A “C₁₋₂₀ hydrocarbon moiety” denotes a hydrocarbon moiety having 1 to 20 carbon atoms.

The term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₅ alkyl” denotes an alkyl group having 1 to 5 carbon atoms. Preferred exemplary alkyl groups are methyl, ethyl, propyl (e.g., n-propyl or isopropyl), or butyl (e.g., n-butyl, isobutyl, sec-butyl, or tert-butyl).

The term “alkylene” refers to an alkanediyl group, i.e. a divalent saturated acyclic hydrocarbon group which may be linear or branched. Unless defined otherwise, the term “alkylene” preferably refers to C₁₋₄ alkylene (including, in particular, linear C₁₋₄ alkylene), and more preferably to methylene or ethylene.

The term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₅ alkenyl” denotes an alkenyl group having 2 to 5 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl).

The term “number average molecular weight” (or “Mₙ”) of a component refers to the average (arithmetic mean) of the molecular weights of the individual molecules of the corresponding component (e.g., of the branched polymer or of the linear polymer). The number average molecular weight (Mₙ) is defined as follows:

$${\overline{M}}_{n} = \frac{{\sum }_{i}^{ } {N}_{i} {M}_{i}}{{\sum }_{i}^{ } {N}_{i}}$$

wherein Nᵢ is the number of molecules of the respective component having a molecular weight Mᵢ, and the summation includes all molecular weights of the corresponding component that are present. The number average molecular weight of a component (such as the branched polymer or the linear polymer) can be determined using methods known in the art, such as e.g., by gel permeation chromatography (GPC), viscosity measurements (viscometry), osmotic-pressure measurements, light-scattering measurements (e.g., using the Zimm method), colligative methods (such as vapor-pressure osmometry, boiling-point elevation, freezing-point depression, or vapor-pressure lowering), end-group determination, or ¹H-NMR. It is preferred that the number average molecular weight is to be determined using GPC or viscosity measurements, more preferably by GPC. The GPC system can be calibrated, e.g., relative to a set of anionically polymerized polystyrenes having a dispersity M_{w}/Mₙ<1.10 (ideally M_{w}/Mₙ=1) as calibration standard. For example, the number average molecular weight can be determined by GPC, using a GPC apparatus C0-8011 (Tosoh Bioscience LLC) equipped with a column GMH_{HR}-H (Tosoh Bioscience LLC), using tetrahydrofuran as the solvent, measuring at 40° C., and using polystyrenes as standard (e.g., as described above). Water can also be used as the solvent instead of tetrahydrofuran in this method. Alternatively, the number average molecular weight can be determined by GPC, using a GPC apparatus 150C (Waters Corporation) equipped with a Shodex Packed Column A-80M (Showa Denko K.K.), measuring at 140° C., using ortho-dichlorobenzene as solvent/carrier, a flow rate of 1.0 mL/min, a sample concentration of about 1 mg/mL, an injection amount of 400 mL, a differential refraction detector, and polystyrenes as standard (e.g., as described above). It is particularly preferred that the number average molecular weight is determined by GPC, using a GPC apparatus 150C (Waters Corporation) equipped with a Shodex Packed Column A-80M (Showa Denko K.K.), measuring at 40° C., using water as solvent, a flow rate of 1.0 mL/min, a sample concentration of about 1 mg/mL, an injection amount of 400 mL, a differential refraction detector, and polystyrenes as standard (e.g., anionically polymerized polystyrenes having a dispersity M_{w}/Mₙ<1.10).

The term “protein” is used herein interchangeably with “polypeptide” or “peptide” and refers to a polymer of two or more amino acids (preferably 10 or more amino acids, more preferably 20 or more amino acids, even more preferably 50 or more amino acids, even more preferably 100 or more amino acids, even more preferably 150 or more amino acids, and yet even more preferably 200 or more amino acids) linked via amide bonds that are formed between an amino group of one amino acid and a carboxyl group of another amino acid. The amino acids comprised in the protein, which are also referred to as amino acid residues, may be selected from the 20 standard proteinogenic α-amino acids (i.e., Ala, Arg, Asn, Asp, Cys, Glu, Gln, Gly, His, Ile, Leu, Lys, Met, Phe, Pro, Ser, Thr, Trp, Tyr, and Val) but also from non-proteinogenic and/or non-standard α-amino acids (such as, e.g., ornithine, citrulline, homolysine, pyrrolysine, or 4-hydroxyproline) as well as β-amino acids (e.g., β-alanine), γ-amino acids and δ-amino acids. Preferably, the amino acid residues comprised in the protein are selected from α-amino acids, more preferably from the 20 standard proteinogenic α-amino acids (which can be present as the L-isomer or the D-isomer, and are preferably all present as the L-isomer). The protein may be unmodified or may be modified, e.g., at its N-terminus, at its C-terminus and/or at a functional group in the side chain of any of its amino acid residues (particularly at the side chain functional group of one or more Lys, His, Ser, Thr, Tyr, Cys, Asp, Glu, and/or Arg residues). Such modifications may include, e.g., the attachment of any of the protecting groups described for the corresponding functional groups in: Wuts PG & Greene TW, Greene's protective groups in organic synthesis, John Wiley & Sons, 2006. Such modifications may also include the covalent attachment of one or more polyethylene glycol (PEG) chains (forming a PEGylated peptide or protein), the glycosylation and/or the acylation with one or more fatty acids (e.g., one or more C₈₋₃₀ alkanoic or alkenoic acids; forming a fatty acid acylated peptide or protein). Moreover, such modification preferably includes the covalent attachment of one or more fluorescent dyes (such as, e.g., any of the non-protein fluorescent dyes mentioned in the present specification, including, e.g., fluorescein, rhodamine, Oregon green, eosin, or Texas red) and/or one or more phosphorescent dyes. It is generally preferred that the protein is unmodified, unless explicitly indicated otherwise. The amino acid residues comprised in the protein may, e.g., be present as a linear molecular chain (forming a linear protein) or may form one or more rings (corresponding to a cyclic protein). The protein may also form oligomers consisting of two or more identical or different molecules. The protein may, e.g., comprise or consist of about 200 to about 800 amino acid residues, or it may have a molecular weight of about 20 kDa to about 800 kDa.

The term “rubber-like material” refers to a material that has the same or similar properties in terms of viscosity and elasticity as a rubber. This term is used herein interchangeably with “elastomeric material”, “elastomeric matrix”, “elastomer”, “rubber material”, “rubber matrix”, “rubber”, or “synthetic rubber”. In particular, wherever the present specification refers to a “rubber-like material”, this is to be understood as relating also to an “elastomeric material” or to an “elastomeric matrix” and, accordingly, the term “rubber-like material” is thus interchangeable with (i.e., can be replaced by) the term “elastomeric material” or the term “elastomeric matrix” throughout the specification.

The term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

The term “about” refers approximately to the indicated numerical value, e.g., to ±10% of the indicated numerical value, and in particular to ±5% of the indicated numerical value. Whenever the term “about” is used, a specific reference to the exact numerical value indicated is also included. For example, the expression “about 100” refers to the range of 90 to 110, in particular the range of 95 to 105, and preferably refers to the specific value of 100. If the term “about” is used in connection with the endpoints of a range, it refers to the range from the lower endpoint −10% of its indicated numerical value to the upper endpoint +10% of its indicated numerical value, in particular to the range from of the lower endpoint −5% to the upper endpoint +5%, and preferably to the range defined by the exact numerical values of the lower endpoint and the upper endpoint. Thus, the expression “about 10 to about 20” refers to the range of 9 to 22, in particular 9.5 to 21, and preferably 10 to 20. If the term “about” is used in connection with the endpoint of an open-ended range, it refers to the corresponding range starting from the lower endpoint −10% or from the upper endpoint +10%, in particular to the range starting from the lower endpoint −5% or from the upper endpoint +5%, and preferably to the open-ended range defined by the exact numerical value of the corresponding endpoint. For example, the expression “at least about 10%” refers to at least 9%, particularly at least 9.5%, and preferably at least 10%.

It is to be understood that the present invention specifically relates to each and every combination of features and embodiments of the processes, products and uses described herein, including any combination of general and/or preferred features/embodiments.

In this specification, a number of documents including patent applications, scientific literature and manufacturers' manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The present invention particularly relates to the following items:
1. A process of preparing a rubber-like material containing a protein immobilized therein, the process comprising the following steps:
   (a) mixing a protein, a branched polymer and a linear polymer in an aqueous solution to form a gel; and
   (b) drying the gel to obtain a rubber-like material containing the protein immobilized therein;
   wherein the branched polymer comprises at least three polymeric branches bound to a central branching unit.
2. A process of preparing a gel, the process comprising:
   (a) mixing a protein, a branched polymer and a linear polymer in an aqueous solution to form a gel;
   wherein the branched polymer comprises at least three polymeric branches bound to a central branching unit.
3. The process of item 1 or 2, wherein the branched polymer has a number average molecular weight of about 200 Da to about 2000 Da.
4. The process of any one of items 1 to 3, wherein said central branching unit comprised in the branched polymer is a C₁₋₂₀ hydrocarbon moiety which is substituted with 3 to 8 substituent groups, wherein said substituent groups are each independently selected from hydroxy, carboxy and amino, optionally wherein one or more carbon atoms comprised in said C₁₋₂₀ hydrocarbon moiety are each independently replaced by an oxygen atom, a nitrogen atom or a sulfur atom, and further wherein each of the at least three polymeric branches is bound to one of the substituent groups of the C₁₋₂₀ hydrocarbon moiety.
5. The process of any one of items 1 to 4, wherein said central branching unit comprised in the branched polymer is a C₃₋₂₀ hydrocarbon moiety which is substituted with 3 to 8 substituent groups, wherein said substituent groups are each independently selected from hydroxy, carboxy and amino, optionally wherein one or more carbon atoms comprised in said C₃₋₂₀ hydrocarbon moiety are each replaced by an oxygen atom, and further wherein each of the at least three polymeric branches is bound to one of the substituent groups of the C₃₋₂₀ hydrocarbon moiety.
6. The process of any one of items 1 to 5, wherein said central branching unit comprised in the branched polymer is a C₃₋₂₀ hydrocarbon moiety which is substituted with 3 to 8 hydroxy groups, optionally wherein one or more carbon atoms comprised in said C₃₋₂₀ hydrocarbon moiety are each replaced by an oxygen atom, and further wherein each of the at least three polymeric branches is bound to one of the hydroxy groups of the C₃₋₂₀ hydrocarbon moiety.
7. The process of any one of items 1 to 4, wherein said central branching unit comprised in the branched polymer is selected from a trimethylolpropane moiety, a trimethylolethane moiety, a trimethylolmethane moiety, a glycerol moiety, a pentaerythritol moiety, a pentaerythrithiol moiety, a diglycerol moiety, a triglycerol moiety, a dipentaerythritol moiety, a tetraglycerol moiety, a pentaglycerol moiety, a tripentaerythritol moiety, a hexaglycerol moiety, a trimethanolamine moiety, a triethanolamine moiety, a triisopropanolamine moiety, a propane-1,2,3-tricarboxylic acid moiety, a citric acid moiety, an isocitric acid moiety, a trimesic acid moiety, a 1,1,1-tris(aminomethyl)propane moiety, a 1,1,1-tris(aminomethyl)ethane moiety, a tris(aminomethyl)methane moiety, a propane-1,2,3-triamine moiety, a tris(2-aminoethyl)amine moiety, and a tris(carboxymethyl)ethylenediamine moiety.
8. The process of any one of items 1 to 7, wherein said central branching unit comprised in the branched polymer is selected from a trimethylolpropane moiety, a trimethylolethane moiety, a trimethylolmethane moiety, and a glycerol moiety.
9. The process of any one of items 1 to 8, wherein said central branching unit comprised in the branched polymer is a trimethylolpropane moiety.
10. The process of any one of items 1 to 9, wherein said at least three polymeric branches comprised in the branched polymer are each independently a poly(alkylene oxide) having a terminal —OH, —OR, —O—CO—R, —CO—O—R, or —CO—N(R)—R group, wherein each R is independently C₁₋₅ alkyl or C₂₋₅ alkenyl.

11. The process of any one of items 1 to 10, wherein said at least three polymeric branches comprised in the branched polymer are each independently a poly(alkylene oxide) having a terminal —OH, —OR or —O—CO—R group, wherein each R is independently C₁₋₅ alkyl or C₂₋₅ alkenyl.
12. The process of any one of items 1 to 11, wherein said at least three polymeric branches comprised in the branched polymer are each independently a poly(ethylene oxide), a poly(propylene oxide), or a copolymer of ethylene oxide and propylene oxide, wherein said poly(ethylene oxide), said poly(propylene oxide) and said copolymer each have a terminal —OH, —OR or —O—CO—R group, wherein each R is independently C₁₋₅ alkyl.
13. The process of any one of items 1 to 12, wherein said at least three polymeric branches comprised in the branched polymer are each independently a poly(ethylene oxide) having a terminal —OH group.
14. The process of any one of items 1 to 13, wherein the branched polymer has 3 to 8 polymeric branches bound to the central branching unit.
15. The process of any one of items 1 to 14, wherein the branched polymer has 3 or 4 polymeric branches bound to the central branching unit.
16. The process of any one of items 1 to 15, wherein the branched polymer has 3 polymeric branches bound to the central branching unit.
17. The process of any one of items 1 to 16, wherein the branched polymer is a trimethylolpropane ethoxylate.
18. The process of item 17, wherein the trimethylolpropane ethoxylate has a number average molecular weight of about 300 Da to about 1200 Da.
19. The process of item 17 or 18, wherein the trimethylolpropane ethoxylate has a number average molecular weight of about 450 Da, about 730 Da, or about 1040 Da.
20. The process of any one of items 17 to 19, wherein the trimethylolpropane ethoxylate has a number average molecular weight of about 450 Da.
21. The process of any one of items 1 to 20, wherein the linear polymer has a number average molecular weight of about 10 kDa to about 10,000 kDa.
22. The process of any one of items 1 to 21, wherein the linear polymer has a number average molecular weight of about 500 kDa to about 7000 kDa.
23. The process of any one of items 1 to 22, wherein the linear polymer is a poly(alkylene oxide) having a terminal group at each of its two ends which is selected independently from —OH, —OR, —O—CO—R, —CO—O—R and —CO—N(R)—R, wherein each R is independently C₁₋₅ alkyl or C₂₋₅ alkenyl, or wherein the linear polymer is a poly(acrylic acid) or a poly(4-styrenesulfonic acid).
24. The process of any one of items 1 to 23, wherein the linear polymer is a poly(alkylene oxide) having a terminal group at each of its two ends which is selected independently from —OH, —OR and —O—CO—R, wherein each R is independently C₁₋₅ alkyl.
25. The process of any one of items 1 to 24, wherein the linear polymer is a poly(ethylene oxide), a poly(propylene oxide) or a copolymer of ethylene oxide and propylene oxide, wherein said poly(ethylene oxide), said poly(propylene oxide) and said copolymer each have a terminal group at each of their ends, which terminal group is selected independently from —OH, —OR and —O—CO—R, wherein each R is independently C₁₋₅ alkyl.
26. The process of any one of items 1 to 25, wherein the linear polymer is a poly(ethylene oxide) having a terminal —OH group at each of its two ends.
27. The process of item 26, wherein the linear polymer has a number average molecular weight of about 5000 kDa.
28. The process of any one of items 1 to 27, wherein the protein is a luminescent protein.
29. The process of any one of items 1 to 27, wherein the protein is an enzyme.

030. The process of any one of items 1 to 27, wherein the protein is a fusion protein comprising a luminescent protein or an enzyme which is fused, optionally via a linker, to an adaptor protein domain.
31. The process of item 28 or 30, wherein the luminescent protein is a fluorescent protein.
32. The process of item 31, wherein the fluorescent protein is selected from green fluorescent protein, enhanced green fluorescent protein, blue fluorescent protein, cyan fluorescent protein, teal fluorescent protein, yellow fluorescent protein, orange fluorescent protein, red fluorescent protein, near-infrared fluorescent protein, mCherry, mStrawberry, mRaspberry, mOrange, mCitrine, tdTomato, mTagBFP, dsRed, UnaG, eqFP611, Dronpa, TagRFPs, KFP, EosFP, Dendra, and IrisFP.
33. The process of item 29 or 30, wherein the enzyme is an oxidoreductase, a transferase, a DNA polymerase, an RNA polymerase, a kinase, a hydrolase, a lyase, an isomerase, or a ligase.
34. The process of item 30 or any one of its dependent items 31 to 33, wherein the adaptor protein domain is selected from SH2 domain, SH3 domain, and PABC domain.
35. The process of any one of items 1 to 34, wherein step (a) comprises first mixing the branched polymer and the linear polymer, subsequently adding the protein in an aqueous solution and mixing the protein, the branched polymer and the linear polymer in the aqueous solution, and optionally adding further aqueous solution during said mixing, to form a gel.
36. The process of any one of items 1 to 34, wherein step (a) comprises providing the protein in an aqueous solution, adding the branched polymer and the linear polymer to the aqueous solution of the protein, mixing the protein, the branched polymer and the linear polymer in the aqueous solution, and optionally adding further aqueous solution during said mixing, to form a gel.
37. The process of any one of items 1 to 36, wherein the branched polymer and the linear polymer are mixed in step (a) in a mass ratio of 3:1 to 20:1.
38. The process of any one of items 1 to 37, wherein the branched polymer and the linear polymer are mixed in step (a) in a mass ratio of 4:1 to 15:1.
39. The process of any one of items 1 to 38, wherein the branched polymer and the linear polymer are mixed in step (a) in a mass ratio of 6:1 to 12:1.
40. The process of any one of items 1 to 39, wherein the protein is employed in step (a) in an amount of about 3 mass-% to about 35 mass-% with respect to the mass of the linear polymer.
41. The process of any one of items 1 to 40, wherein the protein is employed in step (a) in an amount of about 10 mass-% with respect to the mass of the linear polymer.
42. The process of any one of items 1 to 41, wherein the aqueous solution is water or an aqueous buffer solution.
43. The process of any one of items 1 to 42, wherein the aqueous solution is an aqueous buffer solution selected from phosphate buffer, HEPES buffer, Tris buffer, MOPS buffer, MES buffer, TES buffer, CHES buffer, PIPES buffer, CAPS buffer, HEPPS buffer, imidazole buffer, tricine buffer, bicine buffer, glycine buffer, citric acid buffer, and acetic acid buffer.
44. The process of any one of items 1 to 43, wherein the branched polymer and the linear polymer are mixed in step (a) in a mass ratio of about 12:1, and further wherein the total volume of the aqueous solution employed in step (a) is about 15 μl to about 50 μl per mg of linear polymer.
45. The process of item 44, wherein the total volume of the aqueous solution employed in step (a) is about 20 μl to about 40 μl per mg of linear polymer.
46. The process of any one of items 1 to 45, wherein the process does not comprise any step of thermally curing, UV-curing or crosslinking the polymers that are mixed in step (a).
47. The process of any one of items 1 to 45, wherein the process does not comprise any step of covalently crosslinking the polymers that are mixed in step (a).
48. The process of any one of items 1 to 45, wherein the branched polymer and the linear polymer are not covalently crosslinked.
49. The process of any one of items 1 to 45, wherein the branched polymer and the linear polymer are free of covalent crosslinkages.
50. The process of item 1 or any one of its dependent items 3 to 49, wherein in step (b) the gel is partially dehydrated to obtain a rubber-like material containing the protein immobilized therein.
51. The process of item 1 or any one of its dependent items 3 to 50, wherein in step (b) the gel is partially dehydrated via vacuum drying, freeze-drying, drum-drying, spray drying, or sunlight-ambient evaporation.
52. The process of item 1 or any one of its dependent items 3 to 50, wherein in step (b) the gel is partially dehydrated using a vacuum.
53. The process of item 1 or any one of its dependent items 3 to 50, wherein in step (b) the gel is partially dehydrated in a vacuum station/chamber.
54. The process of item 1 or any one of its dependent items 3 to 50, wherein in step (b) the gel is partially dehydrated in a vacuum station/chamber at a pressure of about 1 mbar to about 10 mbar for a period of less than about 1 hour.
55. The process of item 53 or 54, wherein the gel is deposited onto a substrate using a solvent-based technique before it is introduced into the vacuum station/chamber.
56. The process of any one of items 53 to 55, wherein the gel is deposited onto a substrate via doctor-blading, roll-to-roll coating, spin coating, gravure printing or 3D printing before it is introduced into the vacuum station/chamber.
57. The process of item 1 or any one of its dependent items 3 to 56, wherein the rubber-like material is prepared in the form of a film having a thickness of about 10 nm to about 10 mm.
58. The process of item 1 or any one of its dependent items 3 to 57, wherein the rubber-like material is prepared in the form of a film having a thickness of about 10 μm to about 10 mm.
59. A rubber-like material containing a protein immobilized therein, which is obtainable by the process of item 1 or any one of its dependent items 3 to 58.
60. A rubber-like material containing a protein immobilized therein, wherein the rubber-like material comprises a branched polymer and a linear polymer, and wherein the branched polymer comprises at least three polymeric branches bound to a central branching unit.
61. A gel which is obtainable by the process of item 2 or any one of its dependent items 3 to 49.
62. A gel comprising a protein, a branched polymer and a linear polymer, wherein the branched polymer comprises at least three polymeric branches bound to a central branching unit.
63. A process of preparing a rubber-like material deposited on a substrate, the process comprising depositing the rubber-like material of item 59 or 60 onto a substrate.
64. A process of preparing a rubber-like material deposited on a substrate, the process comprising:
   (a) introducing a substrate into the gel of item 61 or 62; and
   (b) drying the gel on the substrate to obtain a rubber-like material deposited on the substrate.
65. The process of item 64, wherein in step (b) the gel is partially dehydrated via vacuum drying, freeze-drying, drum-drying, spray drying, or sunlight-ambient evaporation.
66. The process of item 64, wherein in step (b) the gel is partially dehydrated using a vacuum.
67. The rubber-like material of item 59 or 60, wherein said material is deposited on a substrate.
68. The process of any one of items 63 to 66 or the rubber-like material of item 67, wherein the substrate is a three-dimensional substrate.
69. The process of any one of items 63 to 66 and 68 or the rubber-like material of item 67 or 68, wherein the substrate is selected from carboxymethyl-cellulose, starch, collagen, silica, clay, metal oxide, diatomaceous earth, hydroxyapatite, ceramic, glass, paper, poly(ethylene terephthalate), poly(ethylene naphthalate), poly(imide), poly(carbonate), and combinations thereof.
70. Use of the rubber-like material of item 59 or 60 as a down-converting material for a hybrid light-emitting diode, wherein the protein immobilized in the rubber-like material is a luminescent protein.
71. Use of the rubber-like material of item 59 or 60 as a down-converting cascade energy transfer encapsulation for a hybrid light-emitting diode, wherein the protein immobilized in the rubber-like material is a luminescent protein.
72. A hybrid light-emitting diode comprising a light-emitting diode and a coating, wherein the coating contains one or more layers of a rubber-like material as defined in item 59 or 60.
73. The use of item 70 or 71 or the hybrid light-emitting diode of item 72, wherein said hybrid light-emitting diode is a hybrid white light-emitting diode.
74. In vitro use of the rubber-like material of any one of items 59, 60 and 67 to 69 in diagnosis.
75. Use of the rubber-like material of any one of items 59, 60 and 67 to 69 in a diagnostic device or kit.
76. A diagnostic device or kit comprising the rubber-like material of any one of items 59, 60 and 67 to 69.
77. The use of item 74, wherein the rubber-like material has not been cooled prior to its use in diagnosis.
78. The use of item 74, wherein the rubber-like material has been stored without cooling prior to its use in diagnosis.
79. The use of item 74, wherein the rubber-like material has been stored at a temperature of about 20° C. to about 35° C. prior to its use in diagnosis.
80. The use of item 75 or the diagnostic device or kit of item 76, wherein said device or kit does not comprise any cooling system.
81. The use of any one of items 74, 75 and 77 to 80 or the diagnostic device or kit of item 76 or 80, wherein the protein immobilized in the rubber-like material is an enzyme.
82. The use of item 75, 80 or 81 or the diagnostic device or kit of item 76, 80 or 81, wherein said diagnostic device or kit is a single-use diagnostic device or kit.
83. Use of the rubber-like material of any one of items 59, 60 and 67 to 69 in a bioreactor, wherein the protein immobilized in the rubber-like material is an enzyme.
84. A bioreactor comprising the rubber-like material of any one of items 59, 60 and 67 to 69, wherein the protein immobilized in the rubber-like material is an enzyme.

The present invention furthermore relates to the following embodiments:
1. A process of preparing a rubber-like material containing an active material immobilized therein, the process comprising the following steps:
   (a) mixing an active material, a branched polymer and a linear polymer in a solvent to form a gel; and
   (b) drying the gel to obtain a rubber-like material containing the active material immobilized therein;
   wherein the branched polymer comprises at least three polymeric branches bound to a central branching unit.
2. A process of preparing a gel, the process comprising:
   (a) mixing an active material, a branched polymer and a linear polymer in a solvent to form a gel;
   wherein the branched polymer comprises at least three polymeric branches bound to a central branching unit.
3. The process of embodiment 1 or 2, wherein the active material is a non-protein active material.
4. The process of any one of embodiments 1 to 3, wherein the active material is selected from small molecules, coordination complexes, polymers, quantum dots, and nanoparticles.
5. The process of any one of embodiments 1 to 4, wherein the active material is selected from porphyrins, perylenediimide or derivatives thereof, cumarins, neutral or charged coordination complexes, luminescent polymers, polyfluorenes, poly(1,4-phenylene), poly(1,4-phenylene vinylene), and luminescent nanoparticles.
6. The process of embodiment 5, wherein the active material is a homoleptic or heteroleptic, neutral or charged coordination complex based on a metal core of Pt(II), Pd(I), Au(I), Ru(II), Ir(III), Os(II), Mn(II), Zn(II), Mg(II), Cu(I), or AI(III).
7 The process of embodiment 5, wherein the active material is a coordination complex selected from [Ru(bpy)₃][PF₆][Ir(ppy)₂(bpy)][PF₆] and [Ir(ppy)₂(acac)].
8. The process of embodiment 5, wherein the active material is a luminescent polymer which is poly[(9,9-di-n-octylfluorenyl-2,7-diyl)-alt-(benzo[2,1,3]thiadiazol-4,8-diyl)].
9. The process of embodiment 5, wherein the active material is a nanoparticle selected from silica and ZnO.
10. The process of any one of embodiments 1 to 4, wherein the active material is a light-emitting polymer selected from 2,5-bis(chloromethyl)-1-methoxy-4-(2-ethylhexyloxy)benzene, MDMO-PPV, MEH-PPV, methyl viologen dichloride hydrate, poly[2,5-bis(3′,7′-dimethyloctyloxy)-1,4-phenylenevinylene], poly[9,9-bis-(2-ethylhexyl)-9H-fluorene-2,7-diyl], poly[2-(2′,5′-bis(2″-ethylhexyloxy)phenyI)-1,4-phenylenevinylene], poly{[2-[2′,5′-bis(2″-ethylhexyloxy)phenyl]-1,4-phenylenevinylene]-co-[2-methoxy-5-(2′-ethylhexyloxy)-1,4-phenylenevinylene]}, poly[2,5-bisoctyloxy)-1,4-phenylenevinylene], poly(2,5-bis(1,4,7,10-tetraoxaundecyI)-1,4-phenylenevinylene), poly(3-cyclohexylthiophene-2 ,5-diyl), poly(9,9-di-n-dodecylfluorenyl-2,7-diyl), poly[(9,9-dihexylfluoren-2,7-diyl)-co-(anthracen-9,10-diyl)], poly[(9,9-dihexylfluoren-2,7-diyl)-alt-(2,5-dimethyl-1,4-phenylene)], poly[(9,9-dihexylfluoren-2,7-diyl)-co-(9-ethylcarbazol-2,7-diyl)], poly(9,9-n-dihexyl-2,7-fluorene-alt-9-phenyl-3,6-carbazole), poly(9,9-di-n-hexylfluorenyl-2,7-diyl), poly(2,5-dihexyloxy-1,4-phenylenevinylene), poly(9,9-di-n-octylfluorenyl-2,7-diyl), poly(2,5-dioctylphenylene-1,4-ethynylene), poly(2,5-dioctyl-1,4-phenylenevinylene), poly[5-methoxy-2-(3-sulfopropoxy)-1,4-phenylenevinylene], poly(3-octylthiophene-2,5-diyl) regiorandom, poly[(m-phenylenevinylene)-alt-(2,5-dihexyloxy-p-phenylenevinylene)], poly[(o-phenylenevinylene)-alt-(2-methoxy-5-(2-ethylhexyloxy)-p-phenylenevinylene)], poly[(m-phenylenevinylene)-alt-(2-methoxy-5-(2-ethylhexyloxy)-p-phenylenevinylene)], poly[(p-phenylenevinylene)-alt-(2-methoxy-5-(2-ethylhexyloxy)-p-phenylenevinylene)], poly[tris(2,5-bis(hexyloxy)-1,4-phenylenevinylene)-alt-(1,3-phenylenevinylene)], poly(9-vinylcarbazole), and poly(p-xylene tetrahydrothiophenium chloride).
11. The process of any one of embodiments 1 to 3, wherein the active material is a non-protein dye, which is preferably a fluorescent dye or a phosphorescent dye.
12. The process of any one of embodiments 1 to 3 and 11, wherein the active material is a non-protein fluorescent dye which is selected from 7-aminoactinomycin D, 8-anilinonaphthalene-1-sulfonic acid, an Alexa Fluor dye, an ATTO dye, benzanthrone, bimane, 9,10-bis(phenylethynyl)anthracene, 5,12-bis(phenylethynyl)naphthacene, bisbenzimide, calcein, carboxyfluorescein, carboxyfluorescein diacetate succinimidyl ester, carboxyfluorescein succinimidyl ester, 1-chloro-9,10-bis(phenylethynyl)anthracene, 2-chloro-9,10-bis(phenylethynyl)anthracene, 2-chloro-9,10-diphenylanthracene, coumarin, 4′,6-diamidino-2-phenylindole, 3,3′-dihexyloxacarbocyanine iodide, a DyLight Fluor dye, epicocconone, FIAsH-EDT2, Fluo-3, Fluo-4, a FluoProbes dye, Fura-2, Fura-2-acetoxymethyl ester, a heptamethine dye, IR-780, IR-808, iminocoumarin, Indian yellow, Indo-1, laurdan, Lucifer yellow, a merocyanine, merocyanine 540, Nile red, a perylene, phloxine B, a phycobilin, phycoerythrobilin, phycourobilin, phycoviolobilin, phycocyanobilin, pyranine, a rhodamine, rhodamine B, rhodamine 123, rhodamine 6G, RiboGreen, rubrene, (E)-stilbene, (Z)-stilbene, sulforhodamine 101, sulforhodamine B, SYBR Green I, SYBR Safe, tetraphenyl butadiene, tetrasodium tris(bathophenanthroline disulfonate)ruthenium(II), Texas Red, titan yellow, 6-methoxy-(8-p-toluenesulfonamido)quinoline, umbelliferone, violanthrone, and YOYO-1.
13. The process of any one of embodiments 1 to 3 and 11, wherein the active material is a non-protein fluorescent dye which is selected from xanthene compounds, cyanine compounds, squaraine compounds, naphthalene compounds, coumarin compounds, oxadiazole compounds, anthracene compounds, pyrene compounds, oxazine compounds, acridine compounds, arylmethine compounds, and tetrapyrrole compounds.
14. The process of embodiment 13, wherein the non-protein fluorescent dye is a xanthene compound selected from fluorescein, rhodamine, Oregon green, eosin, and Texas red.
15. The process of embodiment 13, wherein the non-protein fluorescent dye is a cyanine compound selected from cyanine, indocarbocyanine, oxacarbocyanine, thiacarbocyanine, and merocyanine.
16. The process of embodiment 13, wherein the non-protein fluorescent dye is a squaraine compound selected from Seta and SeTau.
17. The process of embodiment 13, wherein the non-protein fluorescent dye is a naphthalene compound which is a dansyl or prodan compound.
18. The process of embodiment 13, wherein the non-protein fluorescent dye is an oxadiazole compound selected from pyridyloxazole, nitrobenzoxadiazole and benzoxadiazole.
19. The process of embodiment 13, wherein the non-protein fluorescent dye is an anthracene compound which is an anthraquinone.
20. The process of embodiment 13 or 19, wherein the anthracene compound is selected from DRAQ5, DRAQ7 and CyTRAK Orange.
21. The process of embodiment 13, wherein the non-protein fluorescent dye is a pyrene compound which is cascade blue.
22. The process of embodiment 13, wherein the non-protein fluorescent dye is an oxazine compound selected from Nile red, Nile blue, cresyl violet, and oxazine 170.
23. The process of embodiment 13, wherein the non-protein fluorescent dye is an acridine compound selected from proflavin, acridine orange, and acridine yellow.
24. The process of embodiment 13, wherein the non-protein fluorescent dye is an arylmethine compound selected from auramine, crystal violet, and malachite green.
25. The process of embodiment 13, wherein the non-protein fluorescent dye is a tetrapyrrole compound selected from porphin, phthalocyanine, and bilirubin.
26. The process of any one of embodiments 1 to 25, wherein the branched polymer has a number average molecular weight of about 200 Da to about 2000 Da.
27. The process of any one of embodiments 1 to 26, wherein said central branching unit comprised in the branched polymer is a C₁₋₂₀ hydrocarbon moiety which is substituted with 3 to 8 substituent groups, wherein said substituent groups are each independently selected from hydroxy, carboxy and amino, optionally wherein one or more carbon atoms comprised in said C₁₋₂₀ hydrocarbon moiety are each independently replaced by an oxygen atom, a nitrogen atom or a sulfur atom, and further wherein each of the at least three polymeric branches is bound to one of the substituent groups of the C₁₋₂₀ hydrocarbon moiety.
28. The process of any one of embodiments 1 to 27, wherein said central branching unit comprised in the branched polymer is a C₃₋₂₀ hydrocarbon moiety which is substituted with 3 to 8 substituent groups, wherein said substituent groups are each independently selected from hydroxy, carboxy and amino, optionally wherein one or more carbon atoms comprised in said C₃₋₂₀ hydrocarbon moiety are each replaced by an oxygen atom, and further wherein each of the at least three polymeric branches is bound to one of the substituent groups of the C₃₋₂₀ hydrocarbon moiety.
29. The process of any one of embodiments 1 to 28, wherein said central branching unit comprised in the branched polymer is a C₃₋₂₀ hydrocarbon moiety which is substituted with 3 to 8 hydroxy groups, optionally wherein one or more carbon atoms comprised in said C₃₋₂₀ hydrocarbon moiety are each replaced by an oxygen atom, and further wherein each of the at least three polymeric branches is bound to one of the hydroxy groups of the C₃₋₂₀ hydrocarbon moiety.
30. The process of any one of embodiments 1 to 27, wherein said central branching unit comprised in the branched polymer is selected from a trimethylolpropane moiety, a trimethylolethane moiety, a trimethylolmethane moiety, a glycerol moiety, a pentaerythritol moiety, a pentaerythrithiol moiety, a diglycerol moiety, a triglycerol moiety, a dipentaerythritol moiety, a tetraglycerol moiety, a pentaglycerol moiety, a tripentaerythritol moiety, a hexaglycerol moiety, a trimethanolamine moiety, a triethanolamine moiety, a triisopropanolamine moiety, a propane-1,2,3-tricarboxylic acid moiety, a citric acid moiety, an isocitric acid moiety, a trimesic acid moiety, a 1,1,1-tris(aminomethyl)propane moiety, a 1,1,1-tris(aminomethyl)ethane moiety, a tris(aminomethyl)methane moiety, a propane-1,2,3-triamine moiety, a tris(2-aminoethyl)amine moiety, and a tris(carboxymethyl)ethylenediamine moiety.
31. The process of any one of embodiments 1 to 30, wherein said central branching unit comprised in the branched polymer is selected from a trimethylolpropane moiety, a trimethylolethane moiety, a trimethylolmethane moiety, and a glycerol moiety.
32. The process of any one of embodiments 1 to 31, wherein said central branching unit comprised in the branched polymer is a trimethylolpropane moiety.
33. The process of any one of embodiments 1 to 32, wherein said at least three polymeric branches comprised in the branched polymer are each independently a poly(alkylene oxide) having a terminal —OH, —OR, —O—CO—R, —CO—O—R, or —CO—N(R)—R group, wherein each R is independently C₁₋₅ alkyl or C₂₋₅ alkenyl.
34. The process of any one of embodiments 1 to 33, wherein said at least three polymeric branches comprised in the branched polymer are each independently a poly(alkylene oxide) having a terminal —OH, —OR or —O—CO—R group, wherein each R is independently C₁₋₅ alkyl or C₂₋₅ alkenyl.
35. The process of any one of embodiments 1 to 34, wherein said at least three polymeric branches comprised in the branched polymer are each independently a poly(ethylene oxide), a poly(propylene oxide), or a copolymer of ethylene oxide and propylene oxide, wherein said poly(ethylene oxide), said poly(propylene oxide) and said copolymer each have a terminal —OH, —OR or —O—CO—R group, wherein each R is independently C₁₋₅ alkyl.
36. The process of any one of embodiments 1 to 35, wherein said at least three polymeric branches comprised in the branched polymer are each independently a poly(ethylene oxide) having a terminal —OH group.
37. The process of any one of embodiments 1 to 36, wherein the branched polymer has 3 to 8 polymeric branches bound to the central branching unit.
38. The process of any one of embodiments 1 to 37, wherein the branched polymer has 3 or 4 polymeric branches bound to the central branching unit.
39. The process of any one of embodiments 1 to 38, wherein the branched polymer has 3 polymeric branches bound to the central branching unit.
40. The process of any one of embodiments 1 to 39, wherein the branched polymer is a trimethylolpropane ethoxylate.
41. The process of embodiment 40, wherein the trimethylolpropane ethoxylate has a number average molecular weight of about 300 Da to about 1200 Da.
42. The process of embodiment 40 or 41, wherein the trimethylolpropane ethoxylate has a number average molecular weight of about 450 Da, about 730 Da, or about 1040 Da.
43. The process of any one of embodiments 40 to 42, wherein the trimethylolpropane ethoxylate has a number average molecular weight of about 450 Da.
44. The process of any one of embodiments 1 to 43, wherein the linear polymer has a number average molecular weight of about 10 kDa to about 10,000 kDa.
45. The process of any one of embodiments 1 to 44, wherein the linear polymer has a number average molecular weight of about 500 kDa to about 7000 kDa.
46. The process of any one of embodiments 1 to 45, wherein the linear polymer is a poly(alkylene oxide) having a terminal group at each of its two ends which is selected independently from —OH, —OR, —O—CO—R, —CO—O—R and —CO—N(R)—R, wherein each R is independently C₁₋₅ alkyl or C₂₋₅ alkenyl, or wherein the linear polymer is a poly(acrylic acid) or a poly(4-styrenesulfonic acid).
47. The process of any one of embodiments 1 to 46, wherein the linear polymer is a poly(alkylene oxide) having a terminal group at each of its two ends which is selected independently from —OH, —OR and —O—CO—R, wherein each R is independently C₁₋₅ alkyl.
48. The process of any one of embodiments 1 to 47, wherein the linear polymer is a poly(ethylene oxide), a poly(propylene oxide) or a copolymer of ethylene oxide and propylene oxide, wherein said poly(ethylene oxide), said poly(propylene oxide) and said copolymer each have a terminal group at each of their ends, which terminal group is selected independently from —OH, —OR and —O—CO—R, wherein each R is independently C₁₋₅ alkyl.
49. The process of any one of embodiments 1 to 48, wherein the linear polymer is a poly(ethylene oxide) having a terminal —OH group at each of its two ends.
50. The process of embodiment 49, wherein the linear polymer has a number average molecular weight of about 5000 kDa.
51. The process of any one of embodiments 1 to 50, wherein step (a) comprises first mixing the branched polymer and the linear polymer, subsequently adding the active material in a solvent and mixing the active material, the branched polymer and the linear polymer in the solvent, and optionally adding further solvent during said mixing, to form a gel.
52. The process of any one of embodiments 1 to 50, wherein step (a) comprises providing the active material in a solvent, adding the branched polymer and the linear polymer to the solvent containing the active material, mixing the active material, the branched polymer and the linear polymer in the solvent, and optionally adding further solvent during said mixing, to form a gel.
53. The process of any one of embodiments 1 to 52, wherein the branched polymer and the linear polymer are mixed in step (a) in a mass ratio of 3:1 to 20:1.
54. The process of any one of embodiments 1 to 53, wherein the branched polymer and the linear polymer are mixed in step (a) in a mass ratio of 4:1 to 15:1.
55. The process of any one of embodiments 1 to 54, wherein the branched polymer and the linear polymer are mixed in step (a) in a mass ratio of 6:1 to 12:1.
56. The process of any one of embodiments 1 to 55, wherein the active material is employed in step (a) in an amount of about 3 mass-% to about 35 mass-% with respect to the mass of the linear polymer.
57. The process of any one of embodiments 1 to 56, wherein the active material is employed in step (a) in an amount of about 10 mass-% with respect to the mass of the linear polymer.
58. The process of any one of embodiments 1 to 57, wherein the solvent is capable of dissolving the active material, the branched polymer and the linear polymer.
59. The process of any one of embodiments 1 to 58, wherein the solvent is an aqueous solution.
60. The process of embodiment 59, wherein the aqueous solution is water or an aqueous buffer solution.
61. The process of embodiment 59 or 60, wherein the aqueous solution is an aqueous buffer solution selected from phosphate buffer, HEPES buffer, Tris buffer, MOPS buffer, MES buffer, TES buffer, CHES buffer, PIPES buffer, CAPS buffer, HEPPS buffer, imidazole buffer, tricine buffer, bicine buffer, glycine buffer, citric acid buffer, and acetic acid buffer.
62. The process of any one of embodiments 1 to 58, wherein the solvent is acetonitrile.
63. The process of any one of embodiments 1 to 62, wherein the branched polymer and the linear polymer are mixed in step (a) in a mass ratio of about 12:1, and further wherein the total volume of the solvent employed in step (a) is about 15 μl to about 50 μl per mg of linear polymer.
64. The process of embodiment 63, wherein the total volume of the solvent employed in step (a) is about 20 μl to about 40 μl per mg of linear polymer.
65. The process of any one of embodiments 1 to 64, wherein the process does not comprise any step of thermally curing, UV-curing or crosslinking the polymers that are mixed in step (a).
66. The process of any one of embodiments 1 to 64, wherein the process does not comprise any step of covalently crosslinking the polymers that are mixed in step (a).
67. The process of any one of embodiments 1 to 64, wherein the branched polymer and the linear polymer are not covalently crosslinked.
68. The process of any one of embodiments 1 to 64, wherein the branched polymer and the linear polymer are free of covalent crosslinkages.
69. The process of embodiment 1 or any one of its dependent embodiments 3 to 68, wherein in step (b) the gel is partially dehydrated to obtain a rubber-like material containing the active material immobilized therein.
70. The process of embodiment 1 or any one of its dependent embodiments 3 to 69, wherein in step (b) the gel is partially dehydrated via vacuum drying, freeze-drying, drum-drying, spray drying, or sunlight-ambient evaporation.
71. The process of embodiment 1 or any one of its dependent embodiments 3 to 69, wherein in step (b) the gel is partially dehydrated using a vacuum.
72. The process of embodiment 1 or any one of its dependent embodiments 3 to 69, wherein in step (b) the gel is partially dehydrated in a vacuum station/chamber.
73. The process of embodiment 1 or any one of its dependent embodiments 3 to 69, wherein in step (b) the gel is partially dehydrated in a vacuum station/chamber at a pressure of about 1 mbar to about 10 mbar for a period of less than about 1 hour.
74. The process of embodiment 72 or 73, wherein the gel is deposited onto a substrate using a solvent-based technique before it is introduced into the vacuum station/chamber.
75. The process of any one of embodiments 72 to 74, wherein the gel is deposited onto a substrate via doctor-blading, roll-to-roll coating, spin coating, gravure printing or 3D printing before it is introduced into the vacuum station/chamber.
76. The process of embodiment 1 or any one of its dependent embodiments 3 to 75, wherein the rubber-like material is prepared in the form of a film having a thickness of about 10 nm to about 10 mm.
77. The process of embodiment 1 or any one of its dependent embodiments 3 to 76, wherein the rubber-like material is prepared in the form of a film having a thickness of about 10 μm to about 10 mm.
78. A rubber-like material containing an active material immobilized therein, which is obtainable by the process of embodiment 1 or any one of its dependent embodiments 3 to 77.
79. A rubber-like material containing an active material immobilized therein, wherein the rubber-like material comprises a branched polymer and a linear polymer, and wherein the branched polymer comprises at least three polymeric branches bound to a central branching unit.
80. A gel which is obtainable by the process of embodiment 2 or any one of its dependent embodiments 3 to 68.
81. A gel comprising an active material, a branched polymer and a linear polymer, wherein the branched polymer comprises at least three polymeric branches bound to a central branching unit.
82. A process of preparing a rubber-like material deposited on a substrate, the process comprising depositing the rubber-like material of embodiment 78 or 79 onto a substrate.
83. A process of preparing a rubber-like material deposited on a substrate, the process comprising:
   (a) introducing a substrate into the gel of embodiment 80 or 81; and
   (b) drying the gel on the substrate to obtain a rubber-like material deposited on the substrate.
84. The process of embodiment 83, wherein in step (b) the gel is partially dehydrated via vacuum drying, freeze-drying, drum-drying, spray drying, or sunlight-ambient evaporation.
85. The process of embodiment 83, wherein in step (b) the gel is partially dehydrated using a vacuum.
86. The rubber-like material of embodiment 78 or 79, wherein said material is deposited on a substrate.
87. The process of any one of embodiments 82 to 85 or the rubber-like material of embodiment 86, wherein the substrate is a three-dimensional substrate.
88. The process of any one of embodiments 82 to 85 and 87 or the rubber-like material of embodiment 86 or 87, wherein the substrate is selected from carboxymethyl-cellulose, starch, collagen, silica, clay, metal oxide, diatomaceous earth, hydroxyapatite, ceramic, glass, paper, poly(ethylene terephthalate), poly(ethylene naphthalate), poly(imide), poly(carbonate), and combinations thereof.
89. Use of the rubber-like material of embodiment 78 or 79 as a down-converting material for a hybrid light-emitting diode, wherein the active material immobilized in the rubber-like material is a non-protein dye.
90. Use of the rubber-like material of embodiment 78 or 79 as a down-converting cascade energy transfer encapsulation for a hybrid light-emitting diode, wherein the active material immobilized in the rubber-like material is a non-protein dye.
91. A hybrid light-emitting diode comprising a light-emitting diode and a coating, wherein the coating contains one or more layers of a rubber-like material as defined in embodiment 78 or 79.
92. The use of embodiment 89 or 90 or the hybrid light-emitting diode of embodiment 91, wherein said hybrid light-emitting diode is a hybrid white light-emitting diode.
93. In vitro use of the rubber-like material of any one of embodiments 78, 79 and 86 to 88 in diagnosis.
94. Use of the rubber-like material of any one of embodiments 78, 79 and 86 to 88 in a diagnostic device or kit.
95. A diagnostic device or kit comprising the rubber-like material of any one of embodiments 78, 79 and 86 to 88.
96. The use of embodiment 93, wherein the rubber-like material has not been cooled prior to its use in diagnosis.
97. The use of embodiment 93, wherein the rubber-like material has been stored without cooling prior to its use in diagnosis.
98. The use of embodiment 93, wherein the rubber-like material has been stored at a temperature of about 20° C. to about 35° C. prior to its use in diagnosis.
99. The use of embodiment 94 or the diagnostic device or kit of embodiment 95, wherein said device or kit does not comprise any cooling system.
100. The use of embodiment 94 or 99 or the diagnostic device or kit of embodiment 95 or 99, wherein said diagnostic device or kit is a single-use diagnostic device or kit.
101. A process of preparing a rubber-like material, the process comprising the following steps:
   (a) mixing a branched polymer and a linear polymer in a solvent to form a gel; and
   (b) drying the gel to obtain a rubber-like material;
   wherein the branched polymer comprises at least three polymeric branches bound to a central branching unit.
102. A process of preparing a gel, the process comprising:
   (a) mixing a branched polymer and a linear polymer in a solvent to form a gel; wherein the branched polymer comprises at least three polymeric branches bound to a central branching unit.
103. The process of embodiment 101 or 102, wherein the branched polymer is as defined in any one or more of embodiments 26 to 43.
104. The process of any one of embodiments 101 to 103, wherein the linear polymer is as defined in any one or more of embodiments 44 to 50.
105. The process of any one of embodiments 101 to 104, wherein said process is further defined by the features recited in any one or more of embodiments 53 to 55 and/or 59 to 68 and/or 70 to 77.
106. A rubber-like material, which is obtainable by the process of embodiment 101 or any one of its dependent embodiments 103 to 105.
107. A gel which is obtainable by the process of embodiment 102 or any one of its dependent embodiments 103 to 105.
108. A process of preparing a rubber-like material deposited on a substrate, the process comprising depositing the rubber-like material of embodiment 106 onto a substrate.
109. A process of preparing a rubber-like material deposited on a substrate, the process comprising:
   (a) introducing a substrate into the gel of embodiment 107; and
   (b) drying the gel on the substrate to obtain a rubber-like material deposited on the substrate.
110. The process of embodiment 109, wherein in step (b) the gel is partially dehydrated via vacuum drying, freeze-drying, drum-drying, spray drying, or sunlight-ambient evaporation.
111. The process of embodiment 109, wherein in step (b) the gel is partially dehydrated using a vacuum.
112. The rubber-like material of embodiment 106, wherein said material is deposited on a substrate.
113. The process of any one of embodiments 108 to 111 or the rubber-like material of embodiment 112, wherein the substrate is a three-dimensional substrate.
114. The process of any one of embodiments 108 to 111 and 113 or the rubber-like material of embodiment 112 or 113, wherein the substrate is selected from carboxymethyl-cellulose, starch, collagen, silica, clay, metal oxide, diatomaceous earth, hydroxyapatite, ceramic, glass, paper, poly(ethylene terephthalate), poly(ethylene naphthalate), poly(imide), poly(carbonate), and combinations thereof.

The invention is also described by the following illustrative figures. The appended figures show:

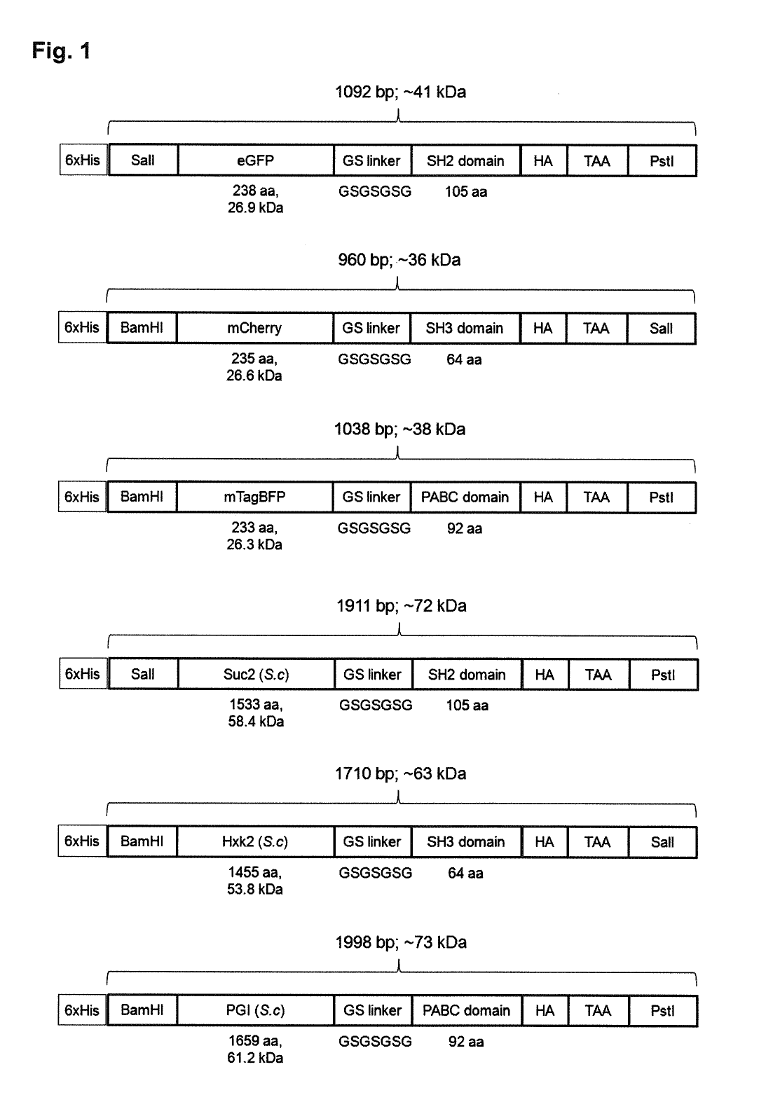

FIG. 1: Reporter constructs for the fluorescent proteins and enzymes used in the examples (see Example 1). GS, glycine-serine amino acid linker; SH2-, SH3-, and PABC-domains represent protein interaction domains; TAA, polyadenylation signal; BamH1 and Sall, restriction sites used for cloning; 6xHis, poly-histidine tag for affinity purification of the fusion proteins. Genes encoding for the enzymes were taken from yeast (Saccharomyces cerevisiae, S.c).

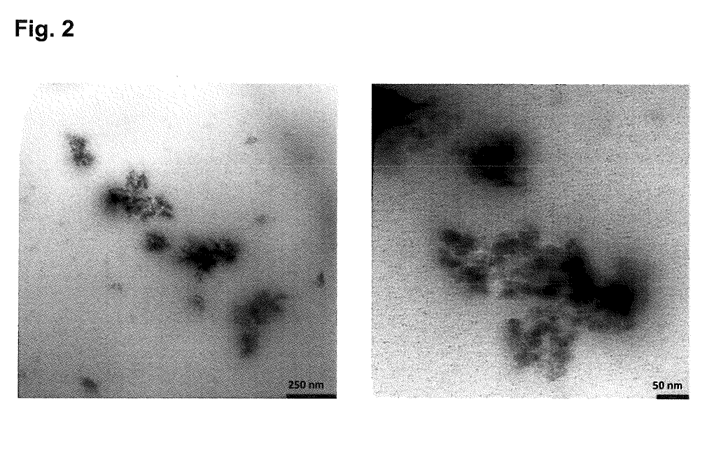

FIG. 2: TEM images of the protein-based gels with low (left) and high (right) magnifications (see Example 1).

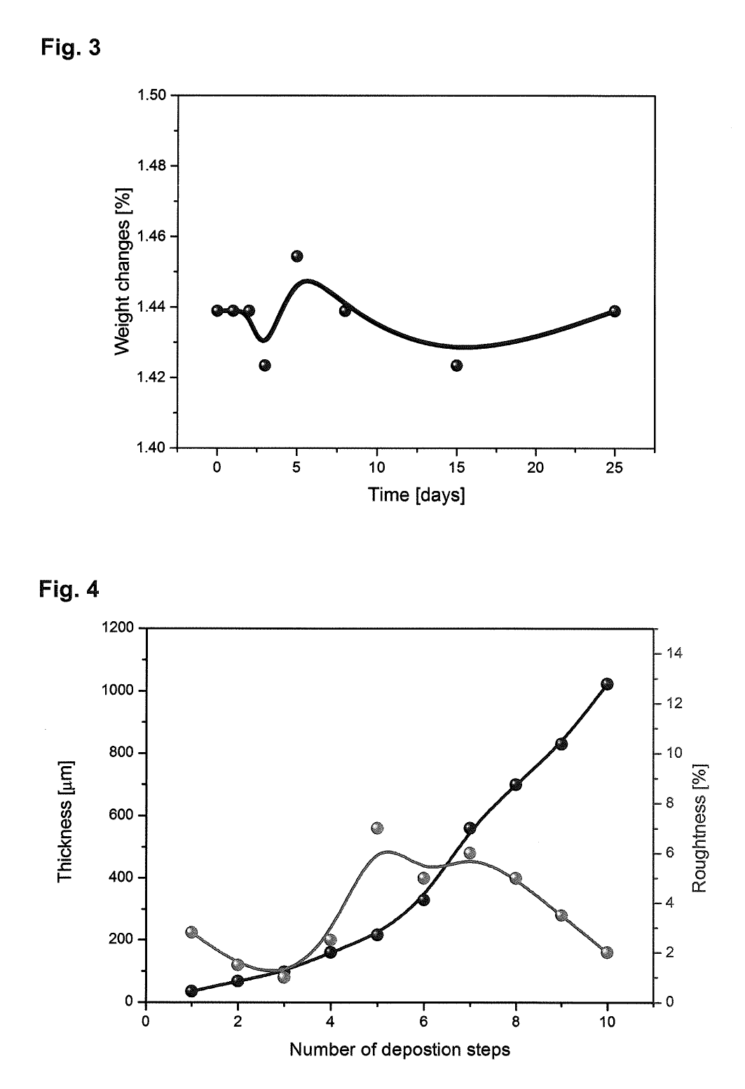

FIG. 3: Water weight changes of the proteins-based films under ambient storage over time (see Example 1).

FIG. 4: Changes of the thickness and roughness of the protein-based films upon repetitive deposition steps (see Example 1).

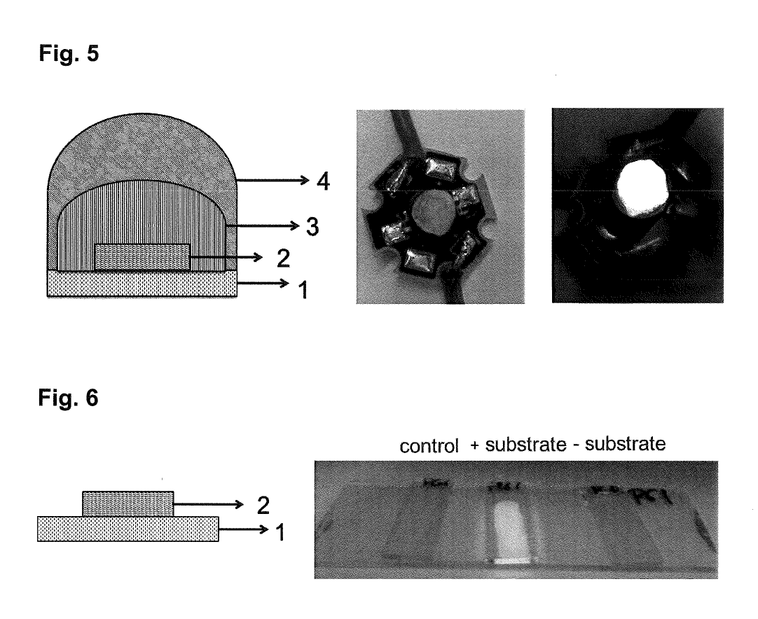

FIG. 5: At the left part the sketch of a hybrid light-emitting diode is shown, in which 1 is the substrate with the electrical connections, 2 is the high-emitting inorganic chip—i.e., in this case blue LED, 3 is the packing or encapsulation system, and 4 is the down-converting encapsulation system that consists of one or several layers of the rubber-like material containing a protein immobilized therein. The right part shows an off and on white hybrid LED.

FIG. 6: At the left part the sketch of a diagnostic device is shown, in which 1 is the substrate and 2 is the rubber-like material containing an enzyme immobilized therein. The right part shows the fluorescence response upon excitation at 310 nm of three different experiments, namely the “control” —i.e., the rubber-like material containing the enzyme immobilized therein, the “+substrate” —i.e., the rubber-like material containing the enzyme immobilized therein, in which 20 μI of the reagent solution were applied, and the “−substrate”—i.e., the rubber-like material containing the enzyme immobilized therein, in which 20 μI of the solution without the reagent were applied.

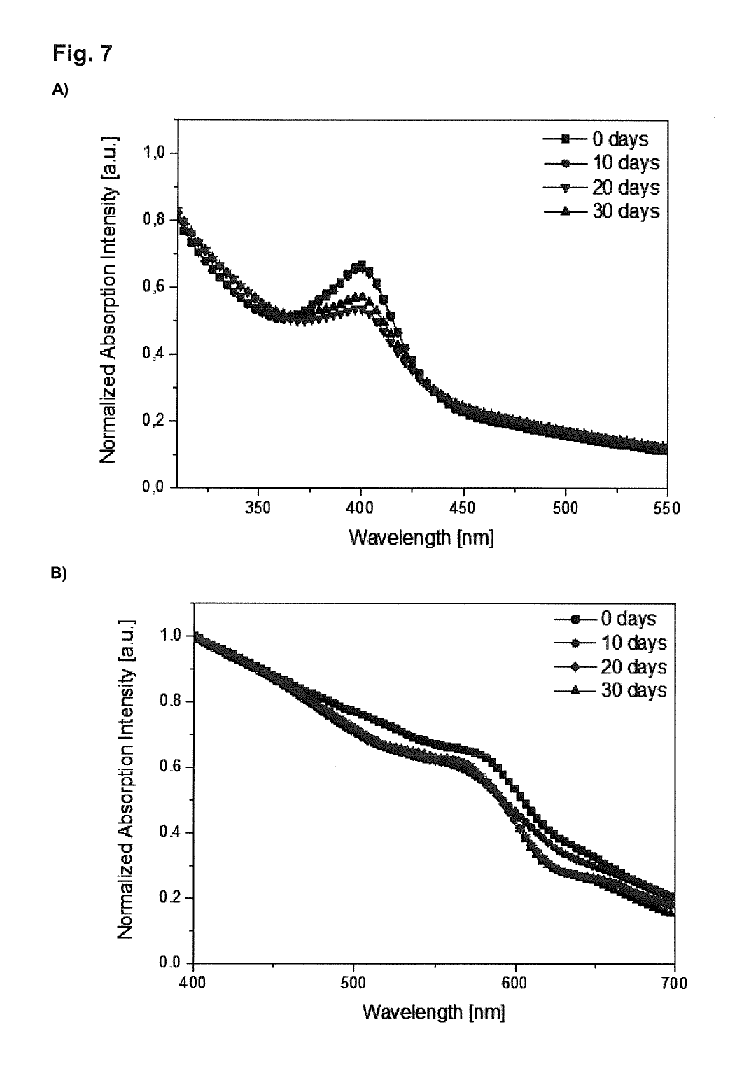

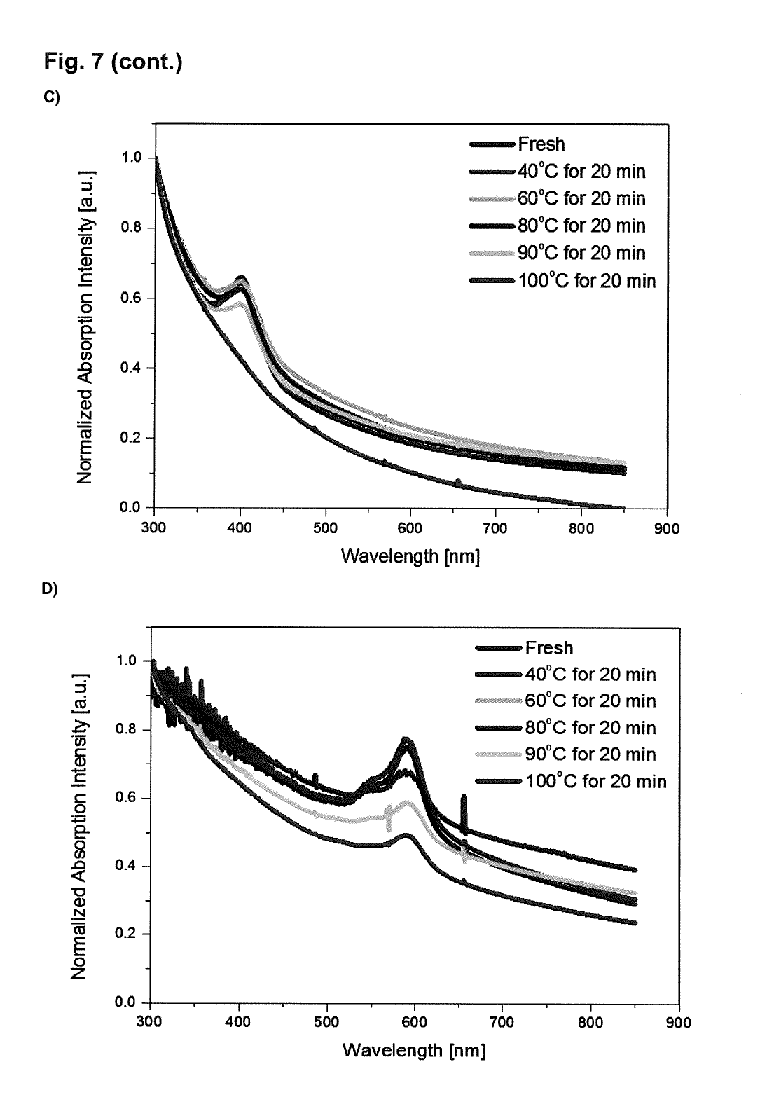

FIG. 7: At the top part the absorption spectra of the mTagBFP (A) and mCherry (B) gels stored under ambient conditions over time are shown. The bottom part shows absorption features of mTagBFP (C) and mCherry (D) gels heated from room temperature to 90° C. with 10° C. steps each for 20 minutes in air.

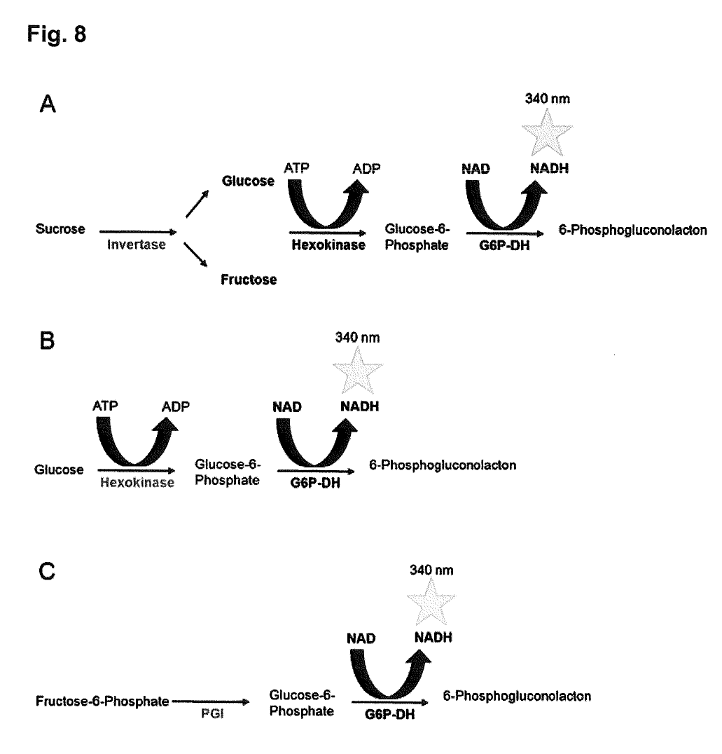

FIG. 8: Principle of the coupled optical tests to determine the activity of the enzymes invertase (A), hexokinase (B) and phosphoglucoisomerase (PGI) (C).

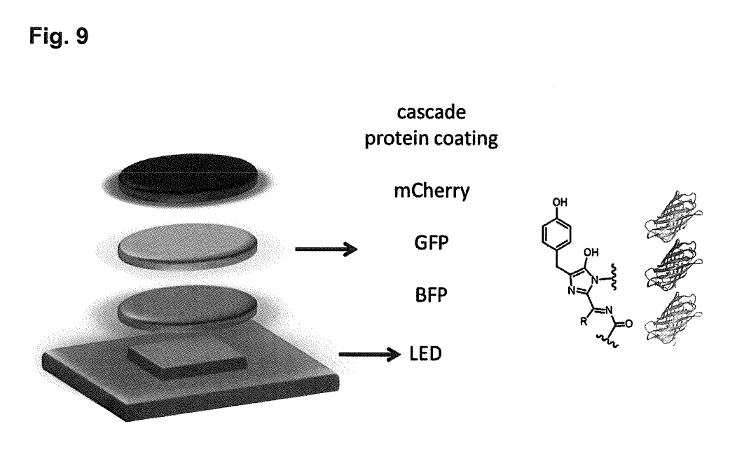

FIG. 9: Representation of a bio-HLED with a cascade coating based on blue, green, and red fluorescent proteins. The structure of the chromophor present in mTagBFP (blue), eGFP (green), and mCherry (red) is shown.

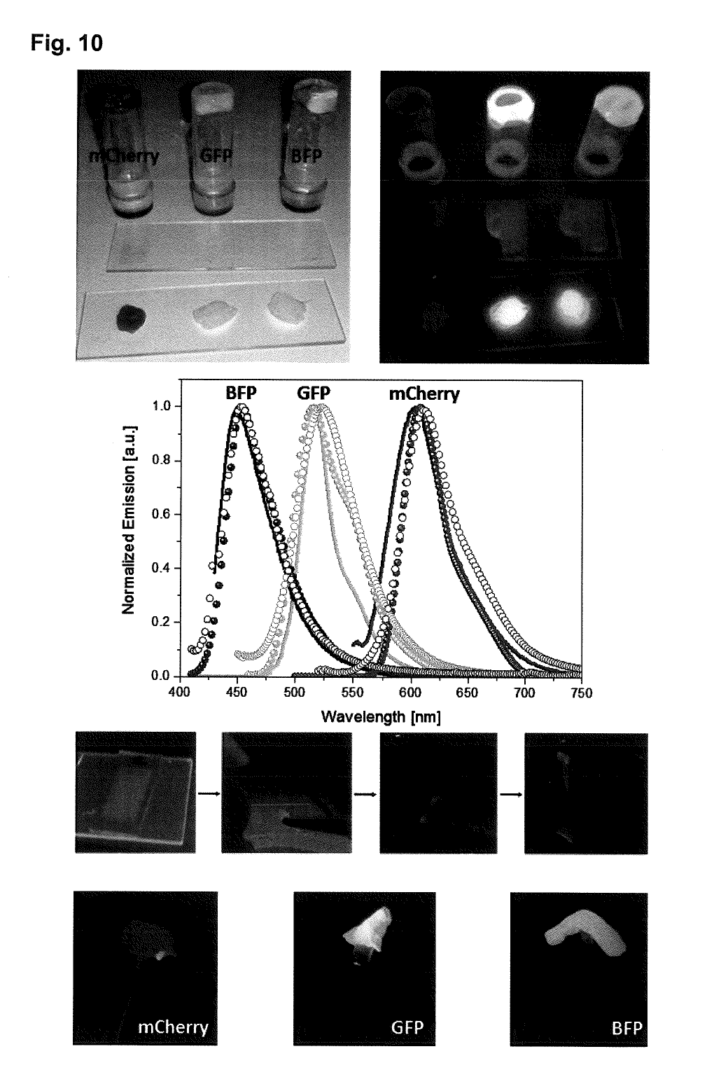

FIG. 10: Upper part—pictures of the protein-based gels and rubber-like materials under ambient (left) and upon excitation at 310 nm (right). Central part—Emission spectra of the three proteins in solution (solid line), gel (open symbols), and rubber-like materials (close symbols) are shown. Lower part—pictures highlighting the easy piling process of the rubber-like material from the glass substrate (top), as well as pictures of the protein-based rubber-like materials with a thickness of 1 mm placed onto a plastic stick by hand (bottom). See Example 1 for further details.

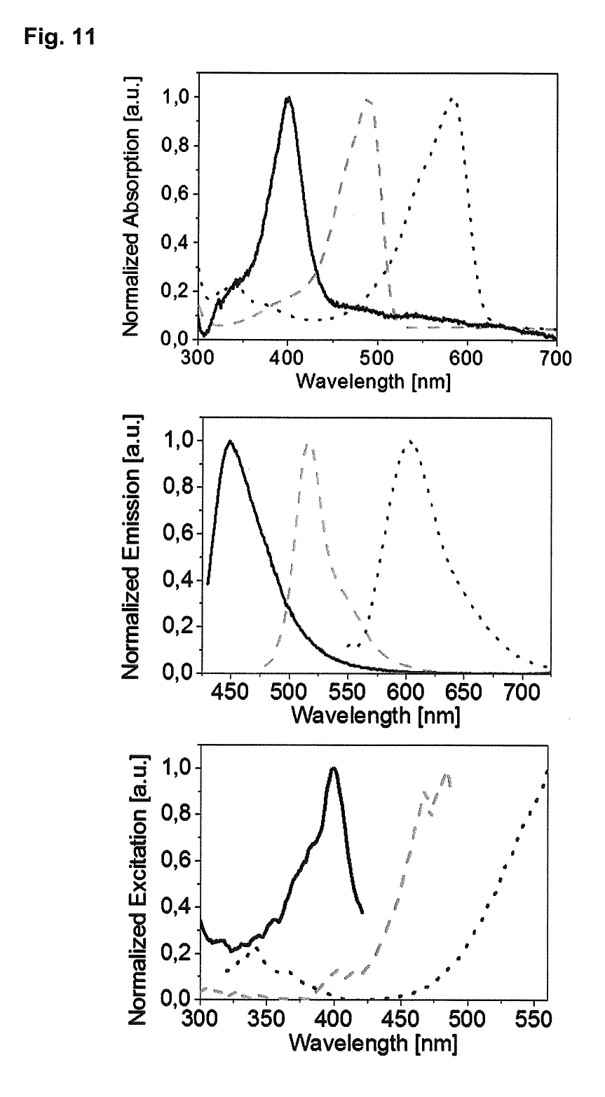

FIG. 11: Normalized absorption (top), emission (central) and excitation (bottom) spectra of mTagBFP (blue), eGFP (green), and mCherry (red) fluorescent proteins in the buffer solution (see Example 1).

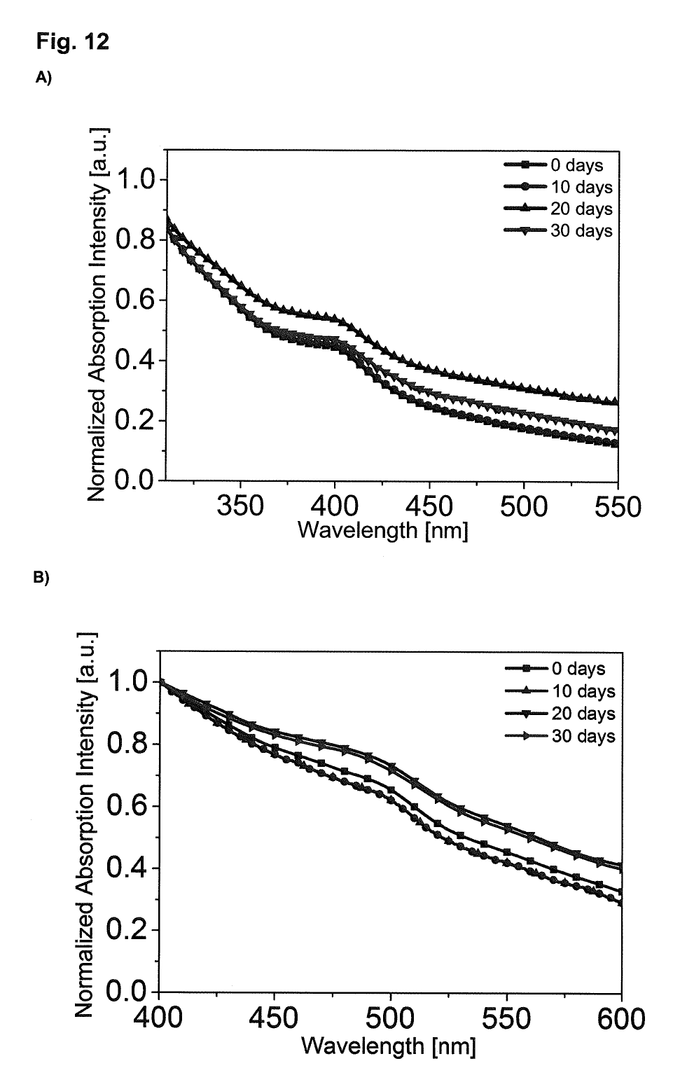

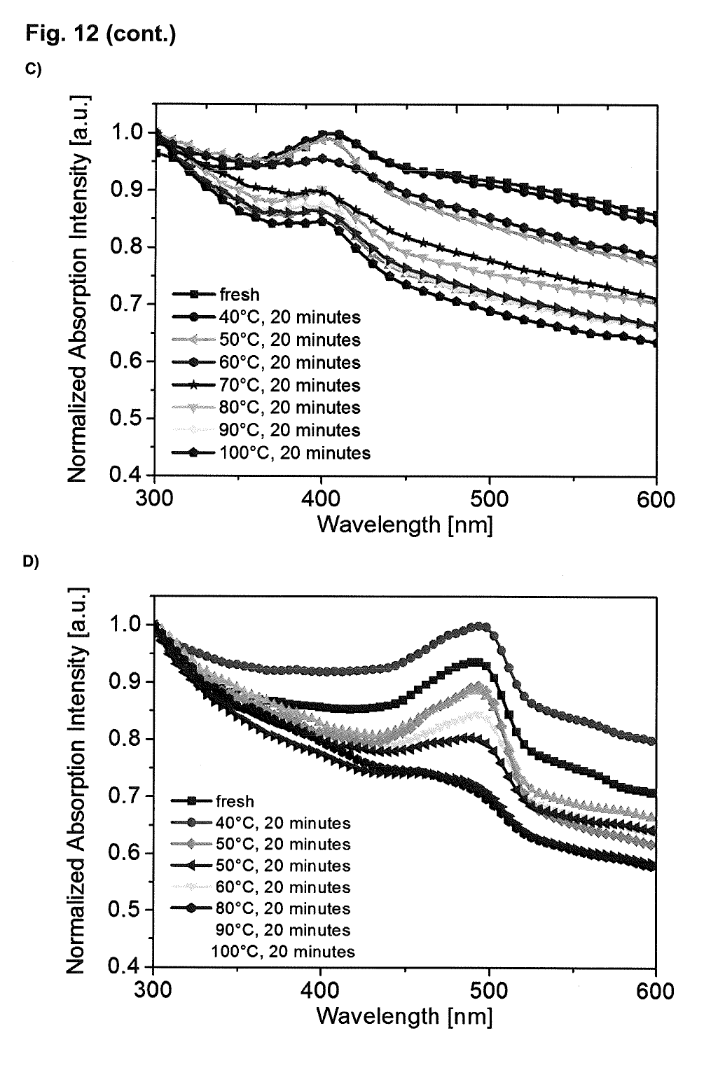

FIG. 12: Changes in the absorption spectrum of blue (A) and green (B) fluorescent protein-based rubbers over time under storage conditions (see Examples 5 and 7). Also shown are the absorption features of the blue (C) and the green (D) fluorescent protein-based rubbers when heated from room temperature to 90° C. with 10° C. steps each for 20 minutes in air.

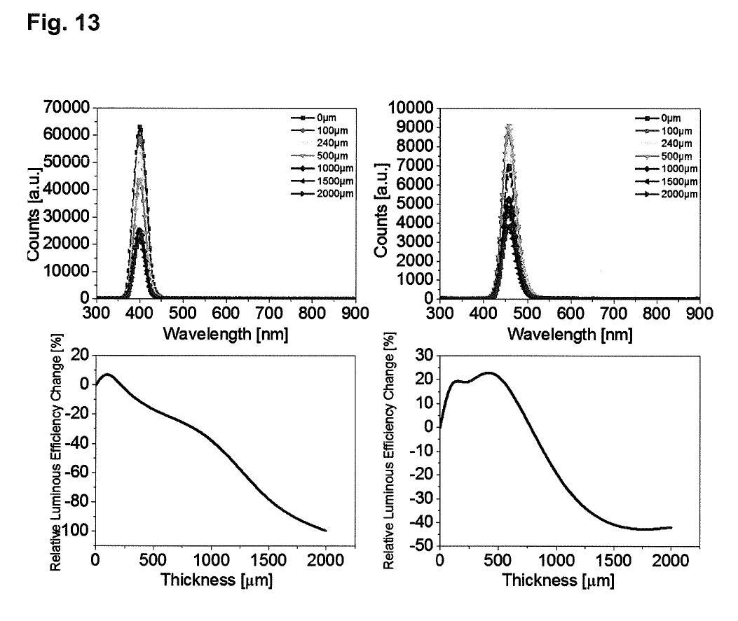

FIG. 13: Changes in the electroluminescence (EL) spectra (top) and relative luminous efficiency (bottom) of UV- (left) and blue-LEDs (right) with a coating lacking fluorescent proteins (see Example 7).

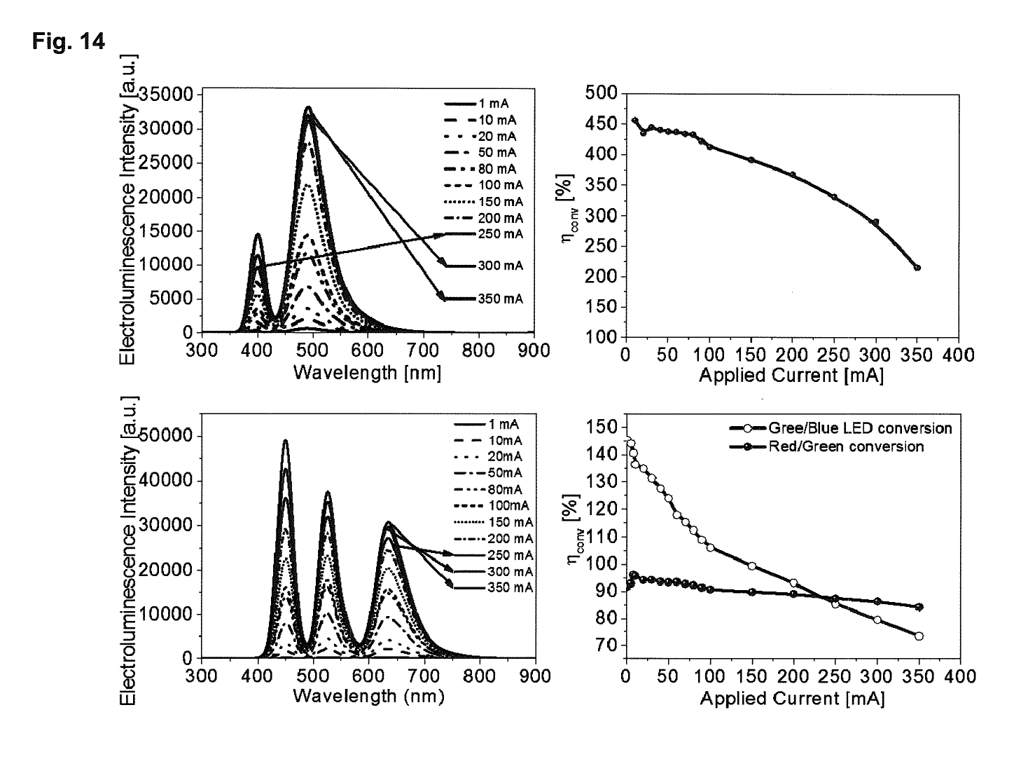

FIG. 14: Electroluminescence spectra and η_{con} versus applied current of UV-LED/mTagBFP (top) and Blue-LED/eGFP/mCherry (bottom).

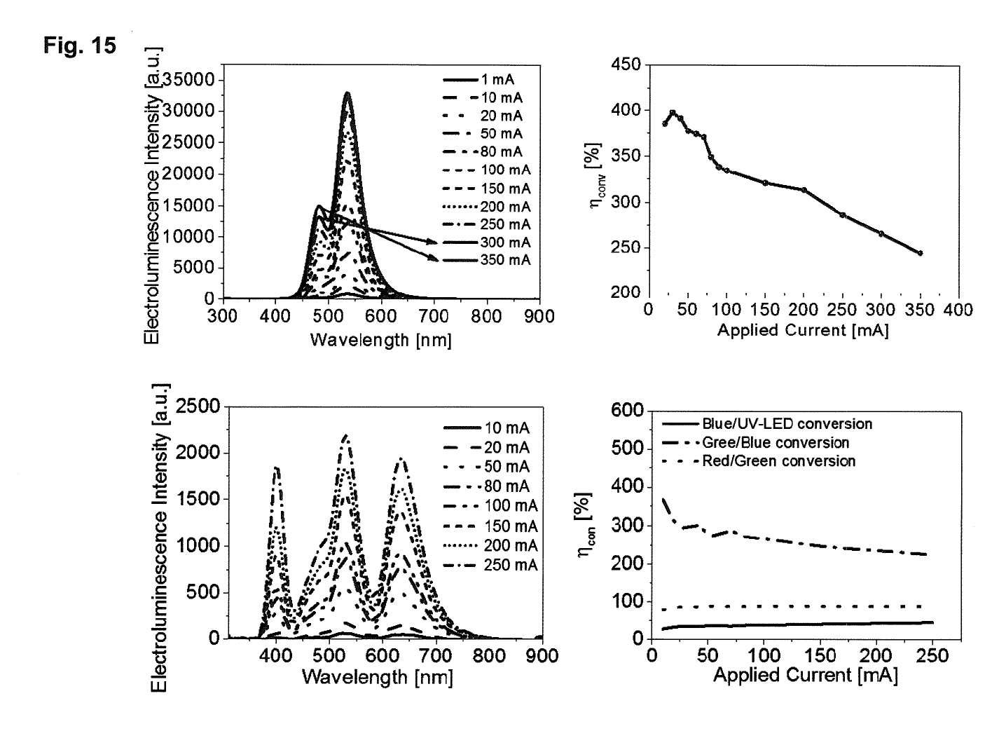

FIG. 15: Electroluminescence spectra and η_{con} versus applied current of blue-LED/eGFP (top) and UV-LED/mTagBFP/eGFP/mCherry (bottom).

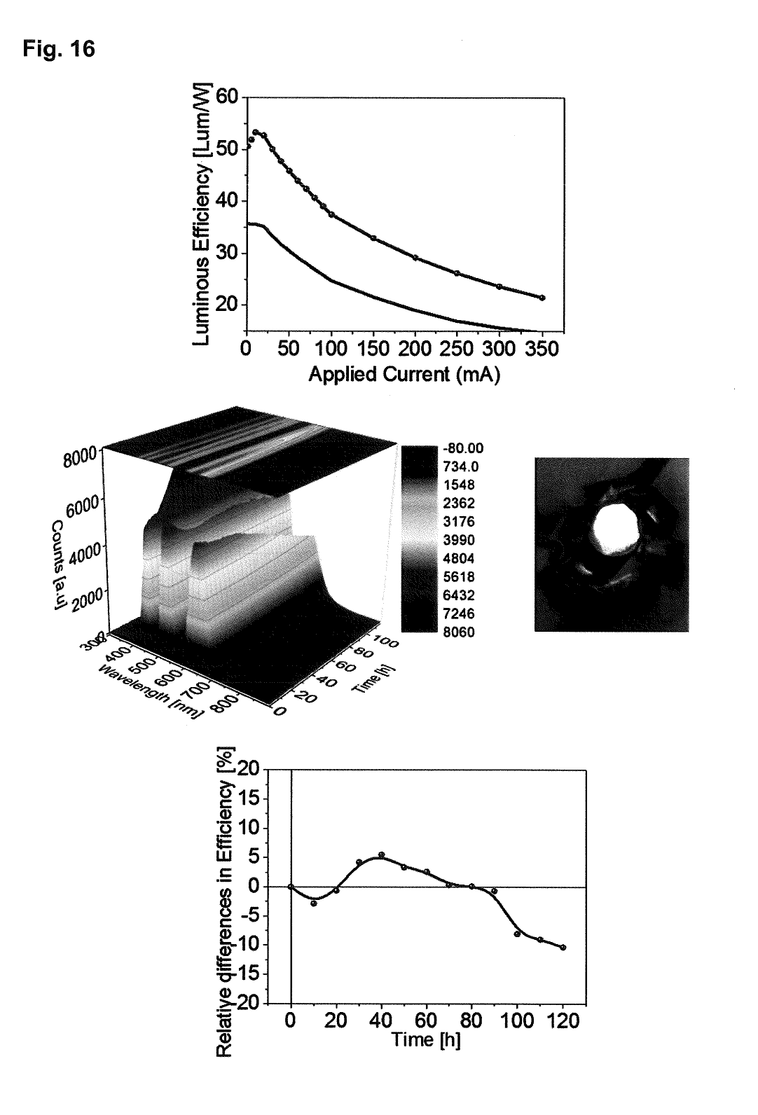

FIG. 16: Upper part—luminous efficiency versus applied currents of architecture 2 (symbols) and the blue-LED (solid line) for comparison purposes. Central part—3D plot showing the changes of the EL spectrum over time (left) at applied current of 10 mA and a picture of a working device with architecture 2 (right). Lower part —relative changes of the luminous efficiency of architecture 2 over time at applied current of 10 mA. See Example 7 for further details.

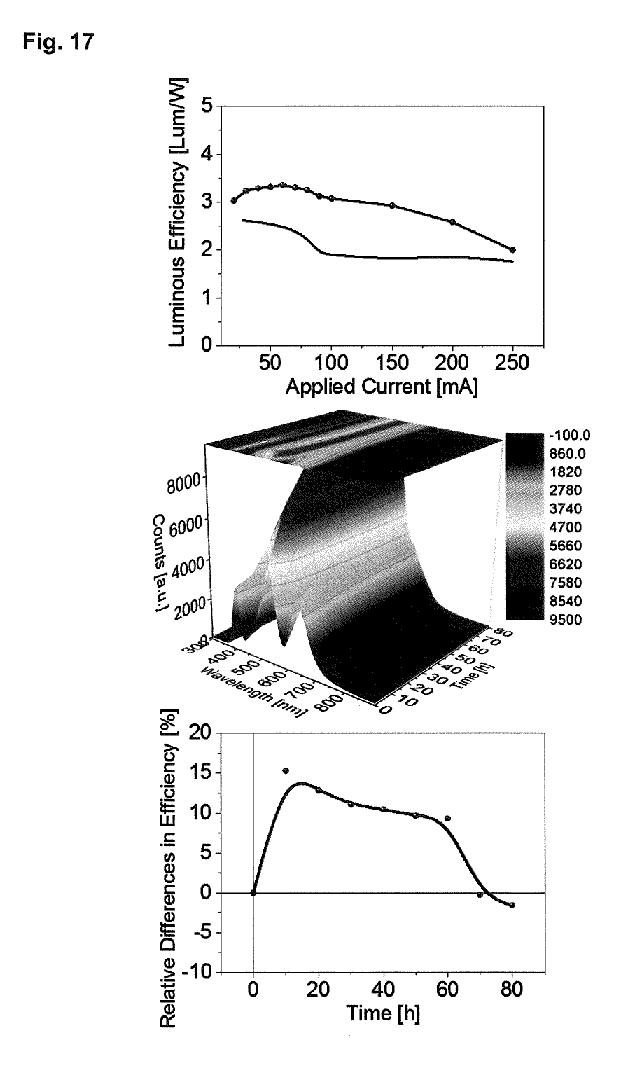

FIG. 17: Upper part—luminous efficiency versus applied currents of architecture 1 (symbol) and the UV-LED (solid line) for comparison purposes. Central part—3D plot showing the changes of the EL spectrum of architecture 1 over time at applied current of 100 mA. Lower part—relative changes of the luminous efficiency of architecture 1 over time at applied current of 100 mA. See Example 7 for further details.

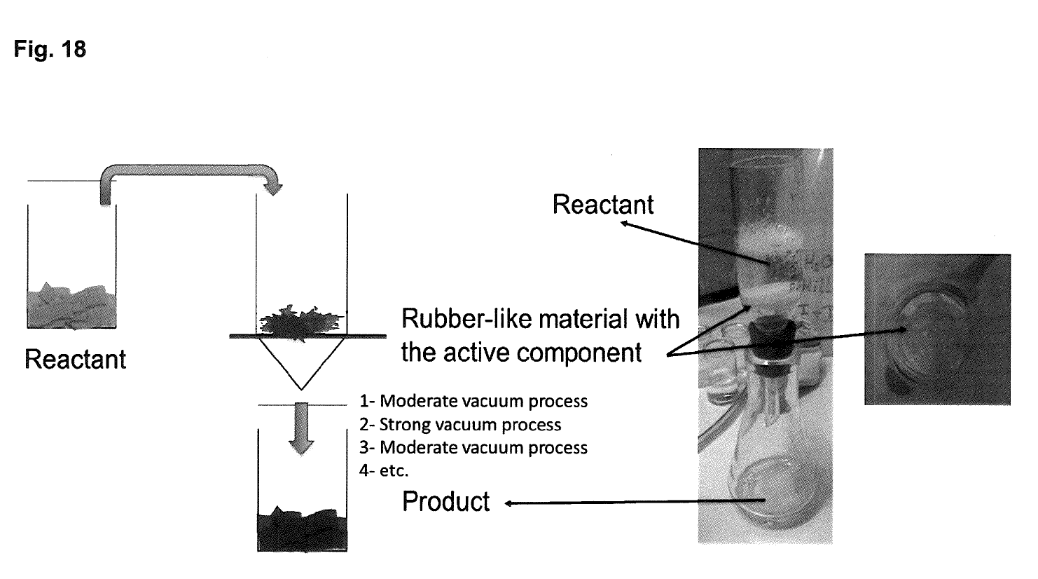

FIG. 18: Design and mechanism of the bioreactor used in Example 8.

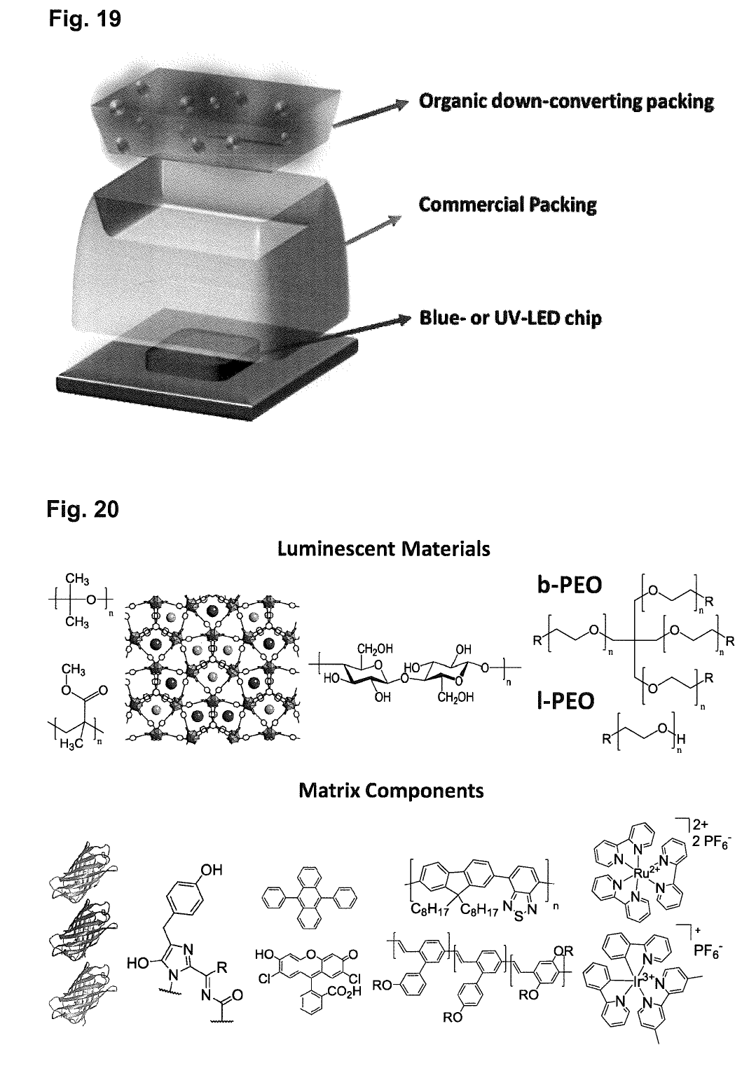

FIG. 19: Sketch of WHLED based on a blue- or UV-LED with organic down-converting packings (see Example 9).

FIG. 20: Upper part—Examples of the components used for the matrix in Example 9—i.e., cross-linked polymers (left), MOF (central left), cellulose (central right), and non-cross-linked branched (b-PEO) and linear (I-PEO) polyethyleneoxide derivatives (right). Lower part—Examples of the luminescent materials in Example 9—i.e., fluorescent proteins (left), small-molecules (central left), polymers (central right), and coordination complexes (right).

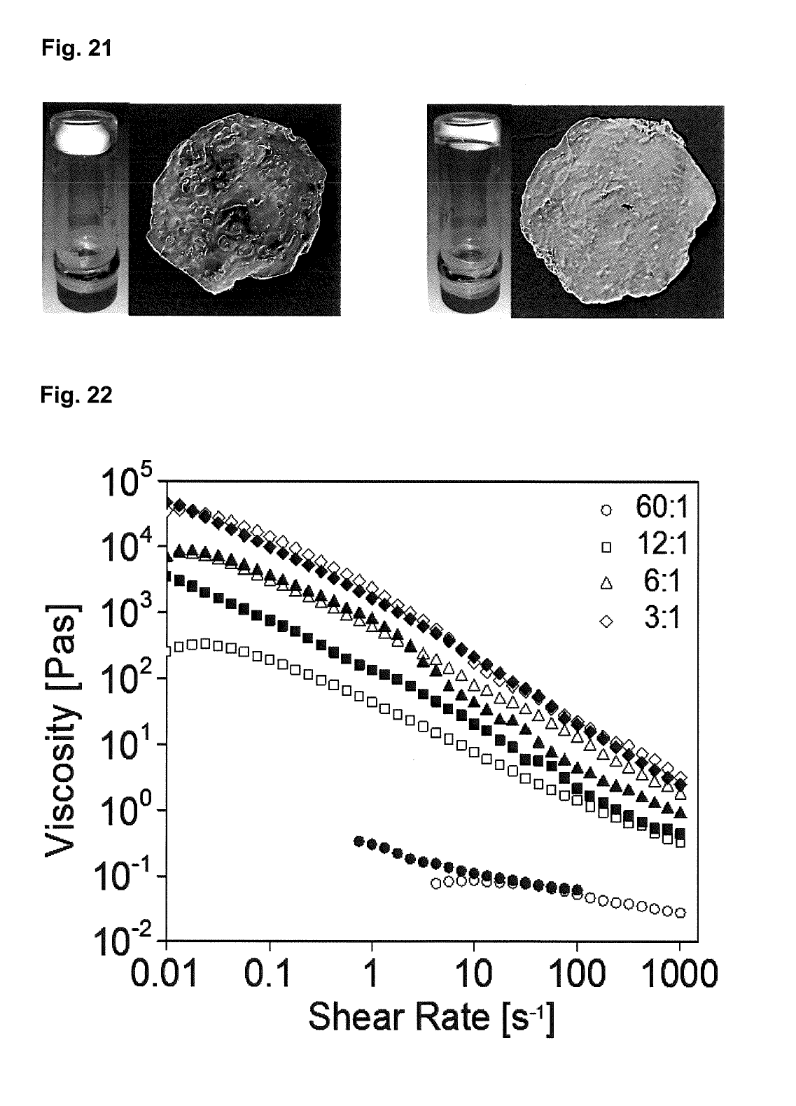

FIG. 21: Pictures of the gels (notice the magnetic stirrer) and rubbers (diameter ˜2.5 cm) prepared with water (left) and acetonitrile (right) with a mixture of b-PEO:I-PEO of 12:1 wt (see Example 9).

FIG. 22: Viscosity functions of water-based (open symbols) and acetonitrile-based (solid symbols) gels with different mass ratios of b-PEO:I-PEO (see Example 9).

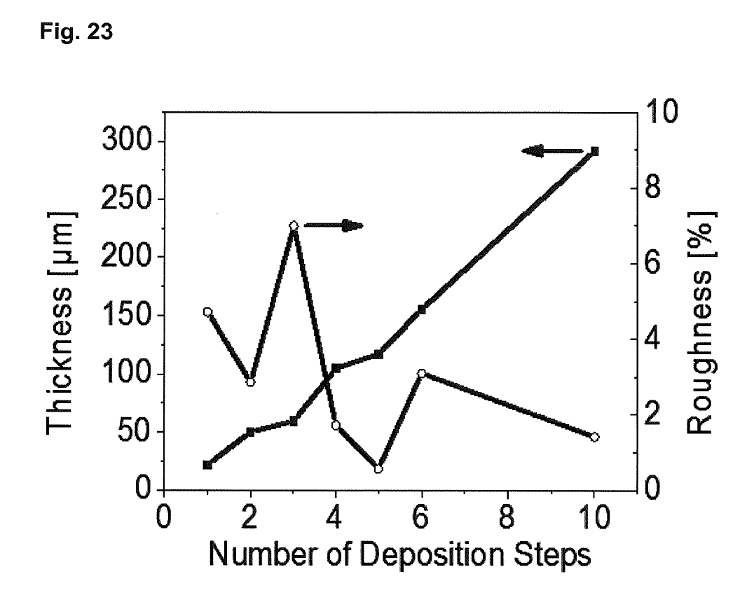

FIG. 23: Changes of the thickness and roughness values of the acetonitrile-based rubbers upon repetitive deposition steps (see Example 9).

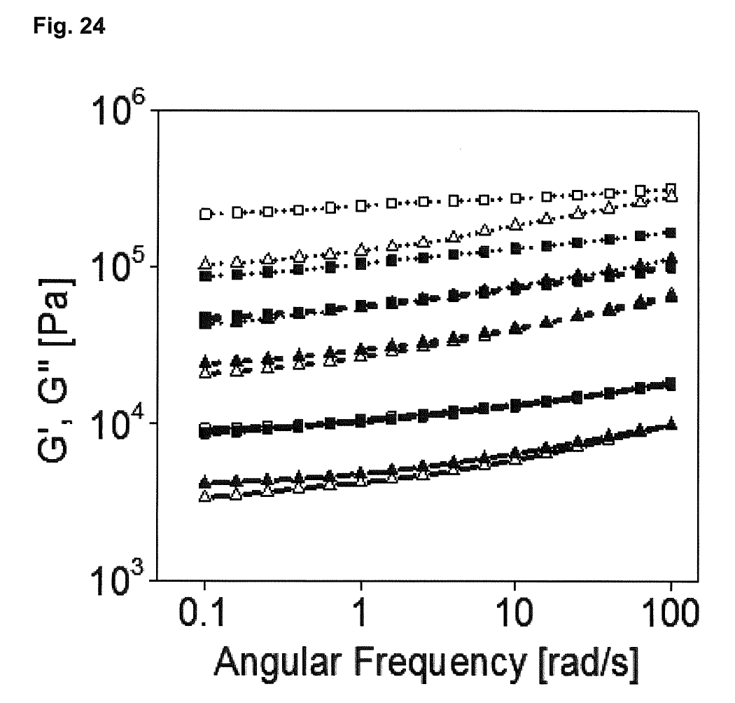

FIG. 24: Storage G′ (square) and loss G″ (triangles) moduli as function of angular frequency for water-based (open symbols) and acetonitrile-based (solid symbols) rubbers at different mass ratios of b-PEO:I-PEO: 12:1 (solid line), 6:1 (dashed line), and 3:1 (dotted line).

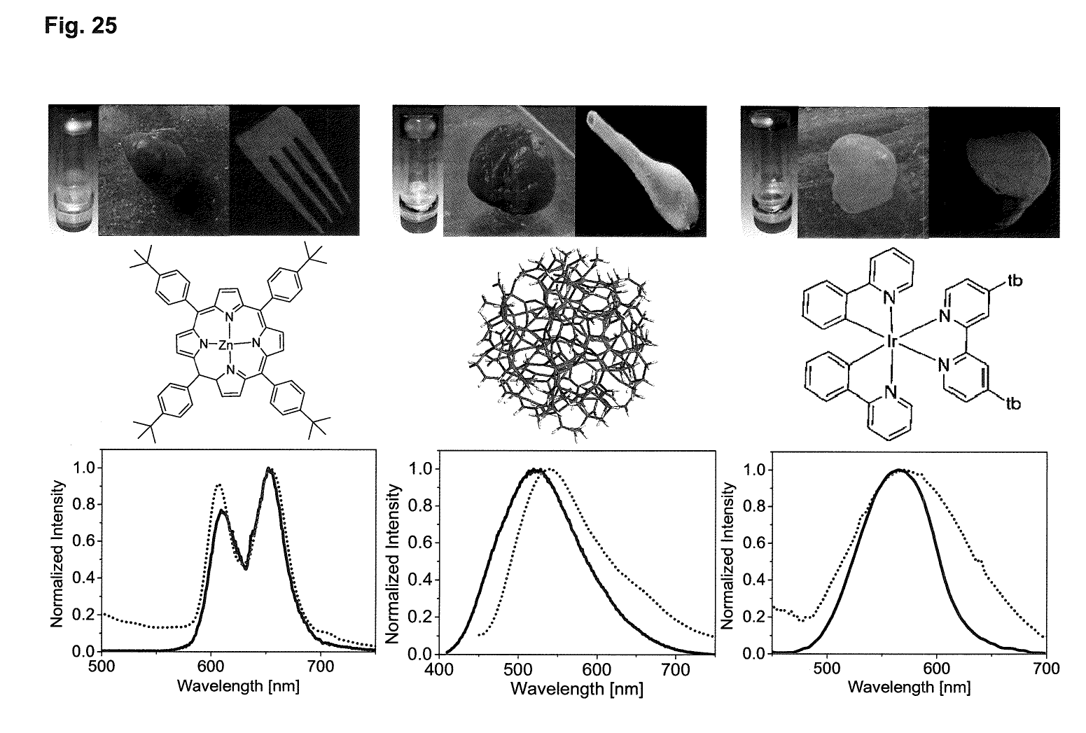

FIG. 25: Upper part—Pictures of examples of the gels (room light, with a magnetic stirrer) and rubber materials with compounds 3, 4, and 7 prepared in a ball-like shape (room light) and onto irregular 3D surfaces (λ_{exc}=310 nm), such as kitchen forks, glass pipette, and plastic vial cap. Central part—Chemical structures of compounds 3, 4, and 7. Bottom part—Emission spectra of the luminescent compounds in solution (solid line) and rubbers (dotted line). See Example 9.

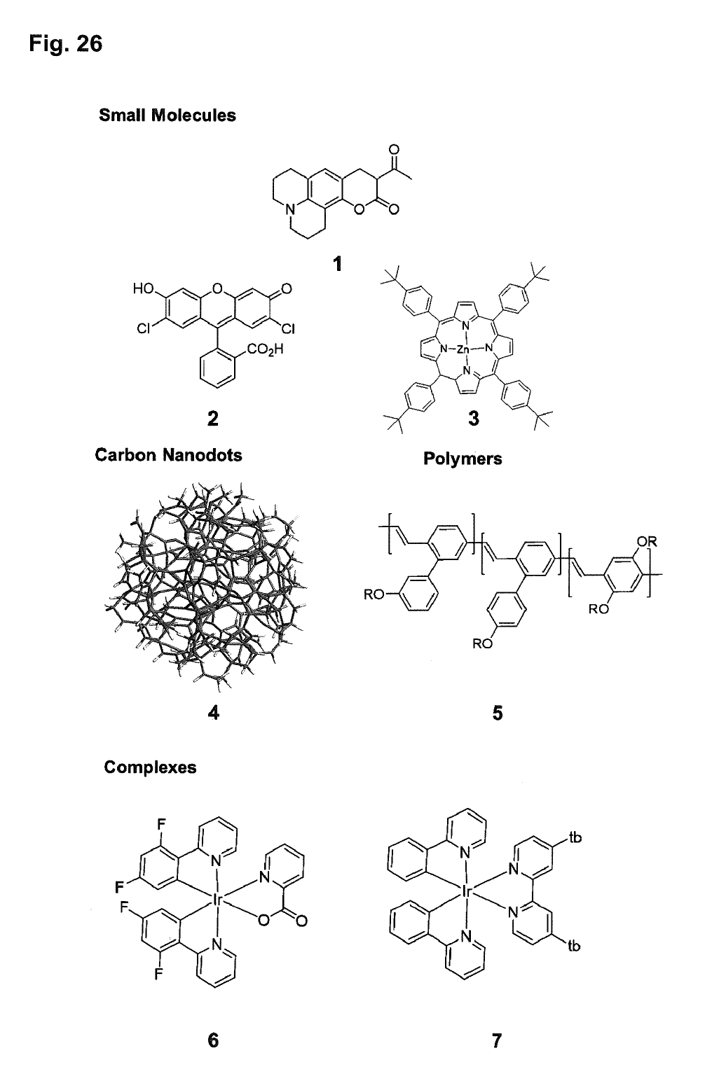

FIG. 26: Chemical structures of the luminescent materials, such as small-molecules (1-3), graphitic quantum dots (4), polymers (5), and coordination complexes (6 and 7), used in Example 9.

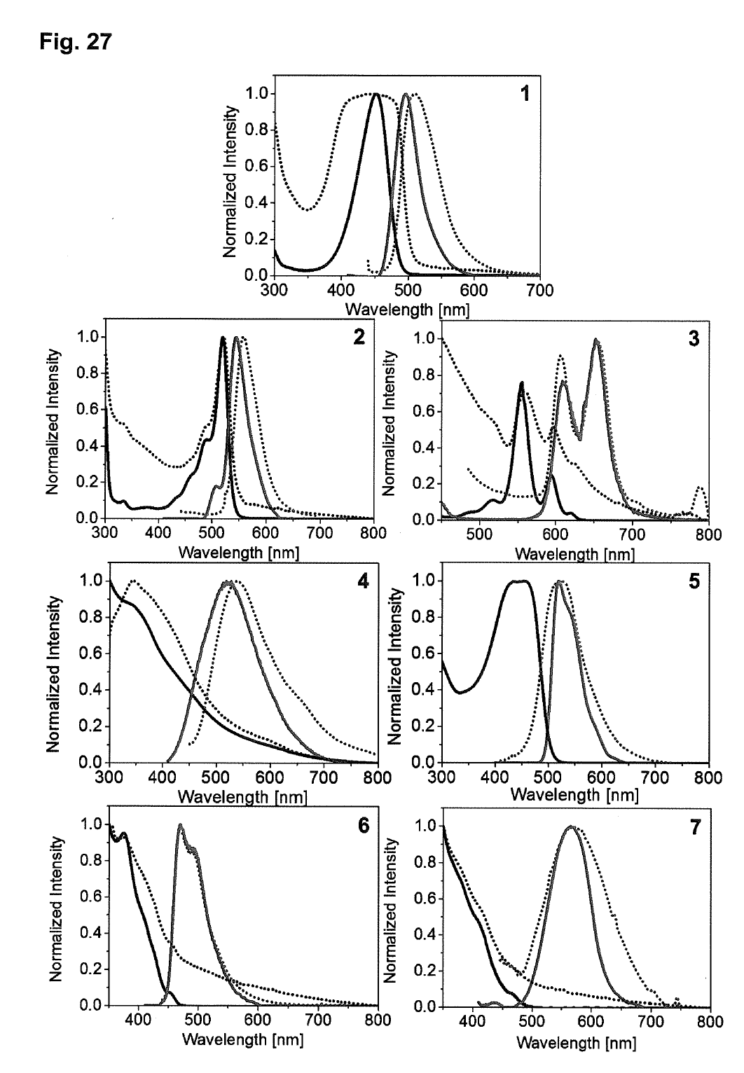

FIG. 27: Absorption (black) and emission (grey) spectra of the luminescent compounds in solution (solid line) and rubbers (dotted line). See Example 9.

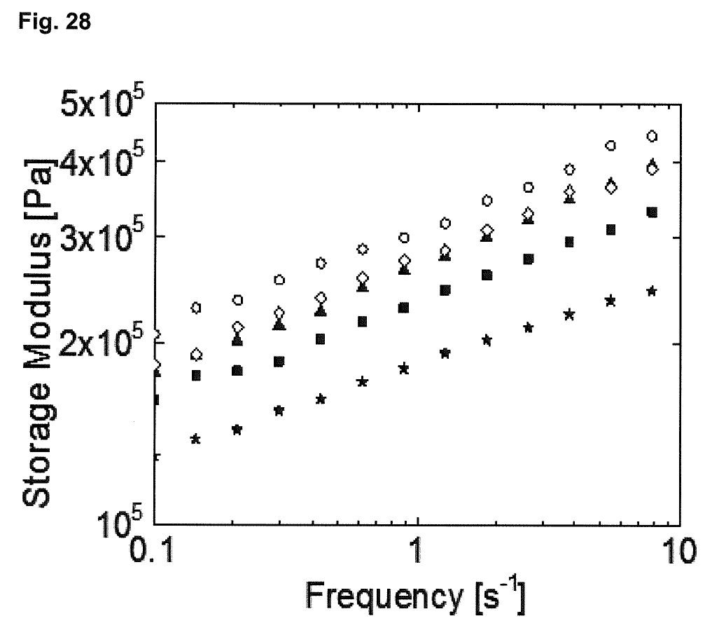

FIG. 28: Frequency sweeps of the storage modulus for different rubbers prepared with b-PEO:I-PEO 6:1 wt. and 1 (diamond), 5 (triangle), and 7 (circle), compared to the references based on water (star) and acetonitrile (square) in Example 9. Note that the differences are caused by variation between samples rather than by the presence of the dopants.

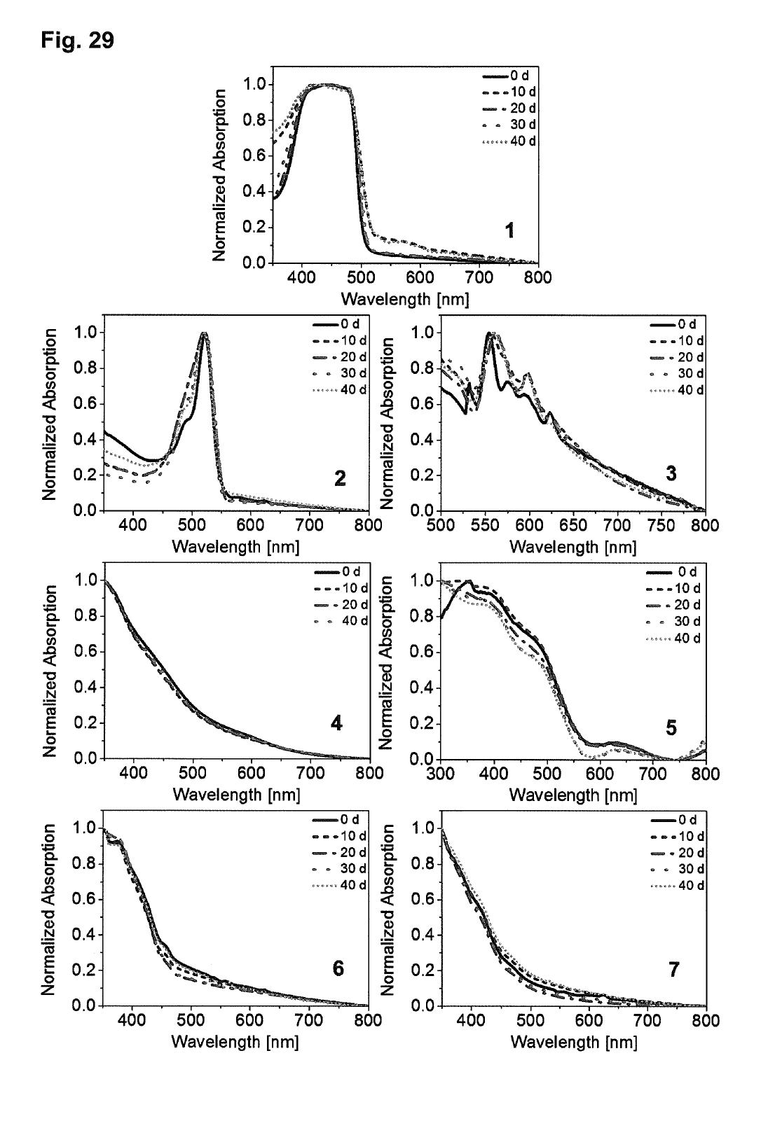

FIG. 29: Changes in the absorption spectra of rubbers based on 1-7 over time under ambient storage conditions. See Example 9.

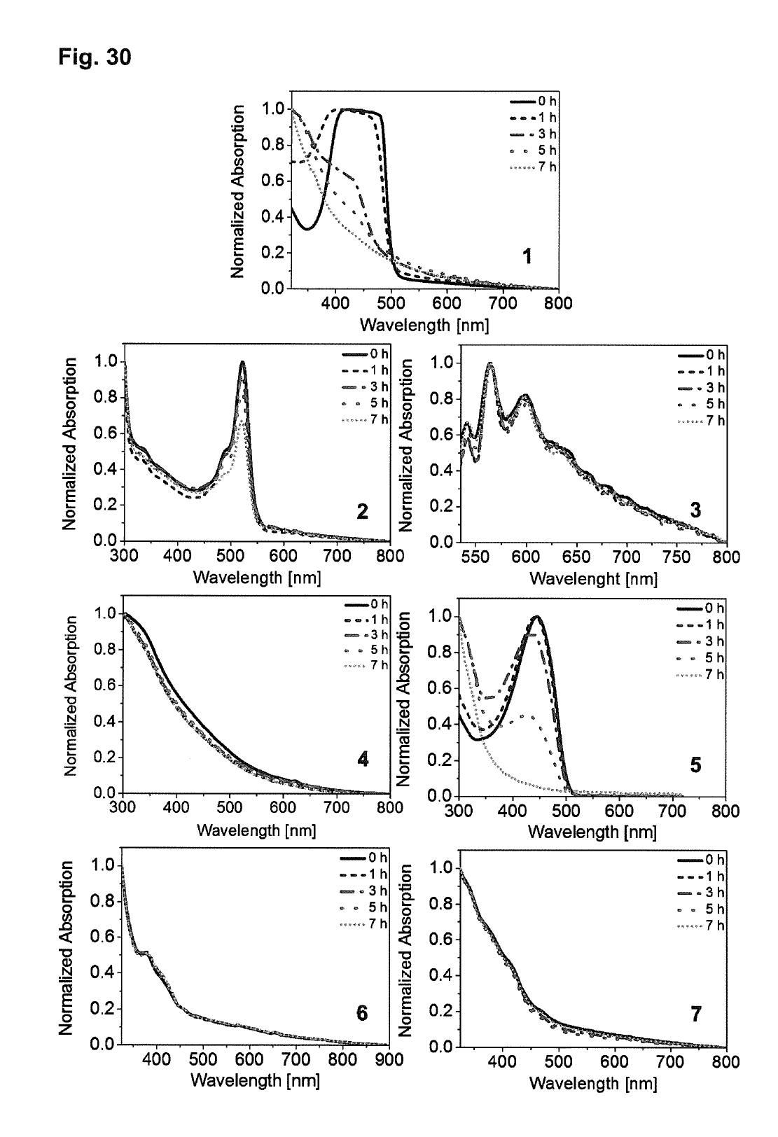

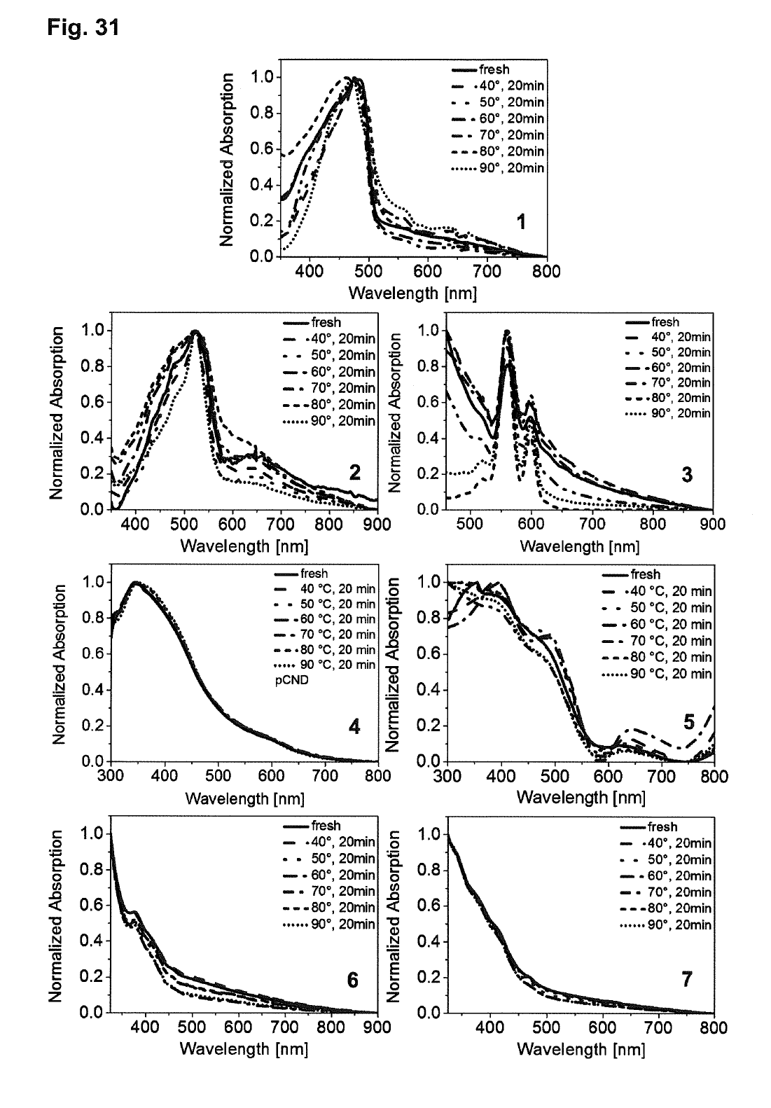

FIG. 30: Changes in the absorption spectra of rubbers based on 1-7 over time upon UV irradiation (310 nm; 8 W) in ambient conditions. See Example 9.

FIG. 31: Changes in the absorption spectra of rubbers based on 1-7 upon heating in ambient conditions. See Example 9.

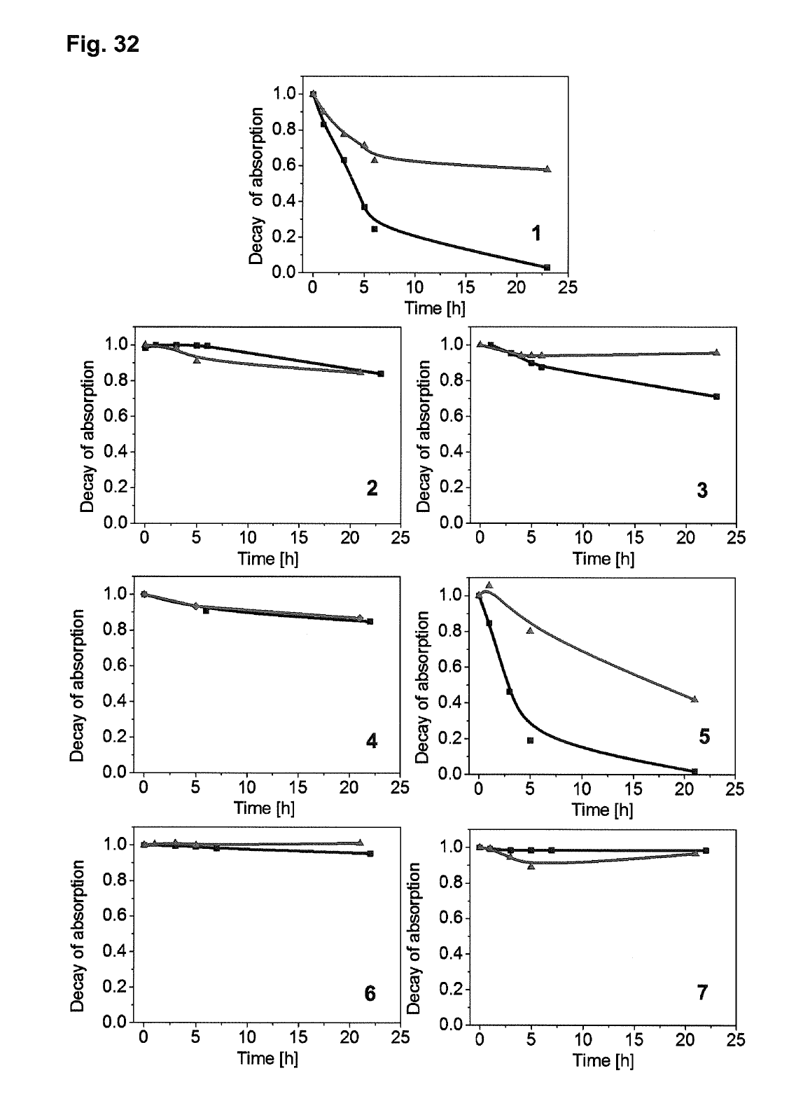

FIG. 32: Comparison of the change in absorption of compounds 1-7 in solution (black squares) and in the rubber (grey triangles). See Example 9.

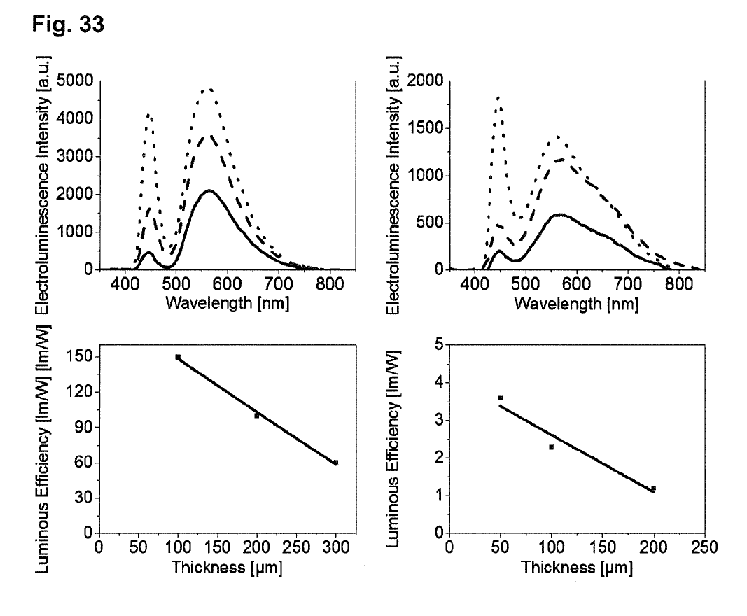

FIG. 33: Upper part—Exemplary electroluminescence spectra of CC- (left) and QD-WHLEDs (right) with three different coating thicknesses—i.e., thicker (solid line), optimum (dashed line), and thinner (dotted line) that related to values of 300/200/100 μm and 200/100/50 μm for CC- (left) and QD-WHLEDs, respectively. Bottom part—Changes of the luminous efficiency upon increasing the coating thickness. See Example 9.

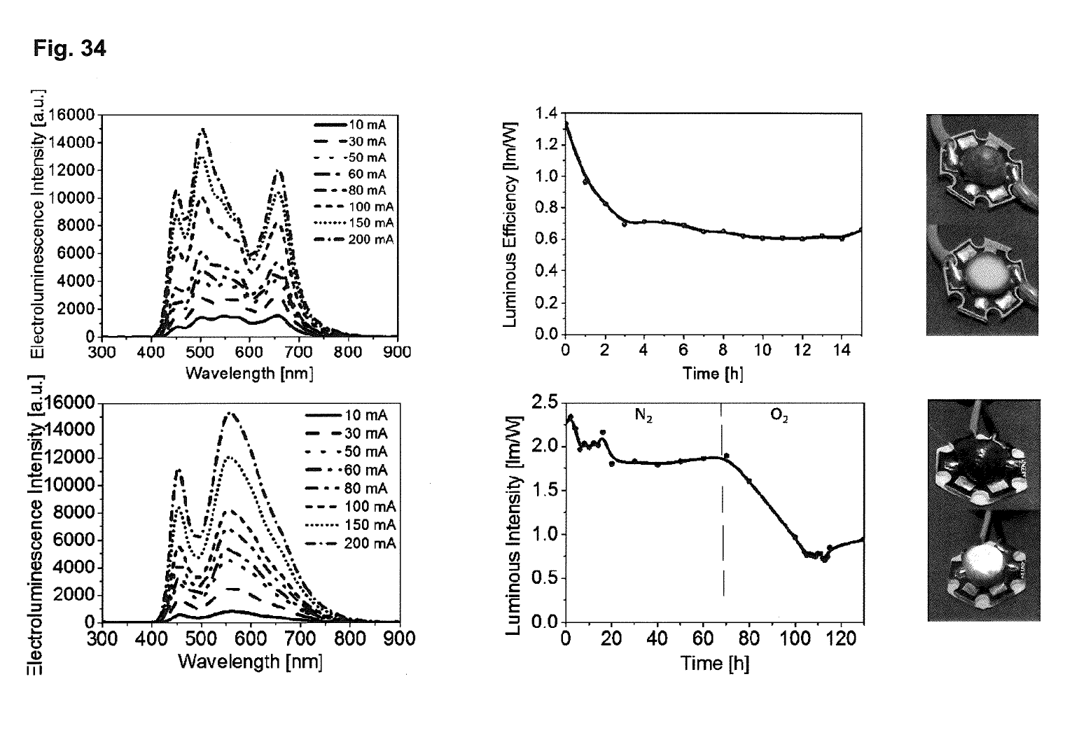

FIG. 34: Electroluminescence spectra of SM-WHLED blue-LED/1/2/3 (top) and QD-WHLED blue-LED/4 (bottom) at different applied currents (left) and the luminous efficiency over time at applied driving current of 10 mA (middle). Pictures of the devices working under ambient conditions are also provided (right). See Example 9.

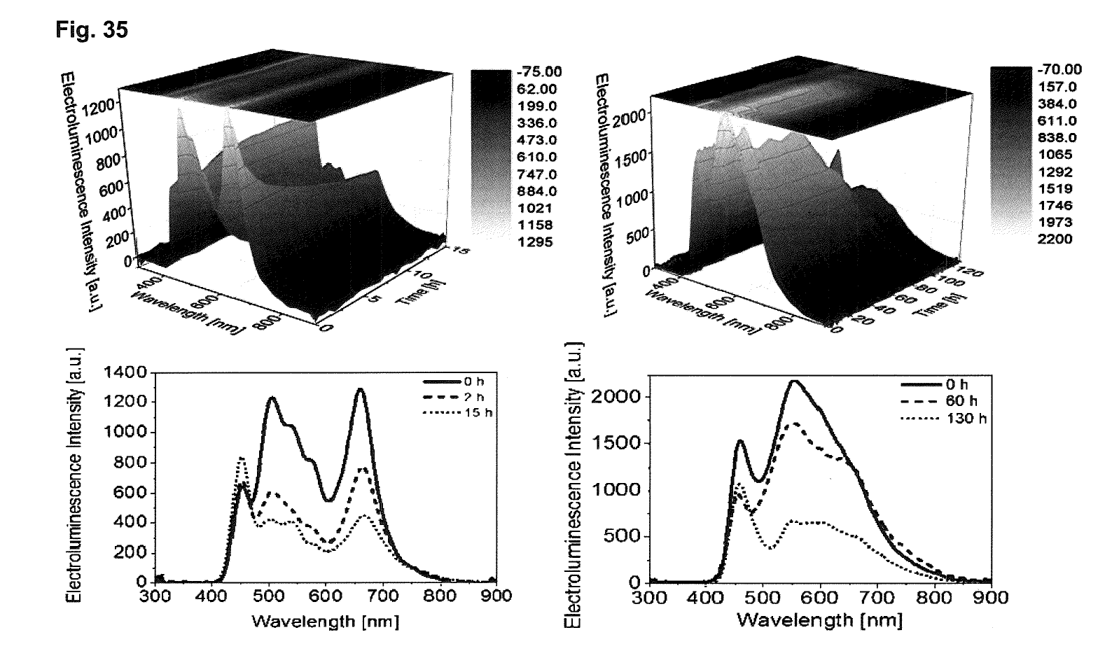

FIG. 35: Changes in the electroluminescence spectrum of SM-WHLED blue-LED/1/2/3 (left) and QD-WHLED blue-LED/4 (right) over time. See Example 9.

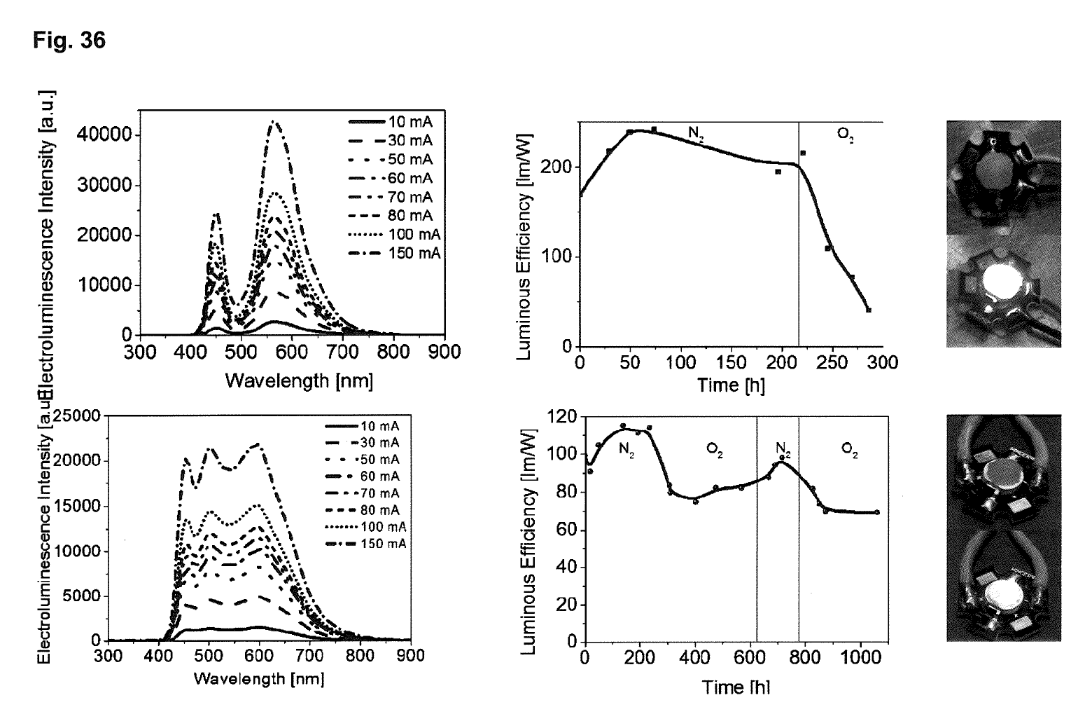

FIG. 36: Upper part—Electroluminescence spectra of P-WHLED blue-LED/5 (top) and CC-WHLED blue-LED/6/7 (bottom) at different applied currents (left) and the luminous efficiency over time at applied driving current of 10 mA (right). Pictures of the devices working under ambient conditions are also provided (right). See Example 9.

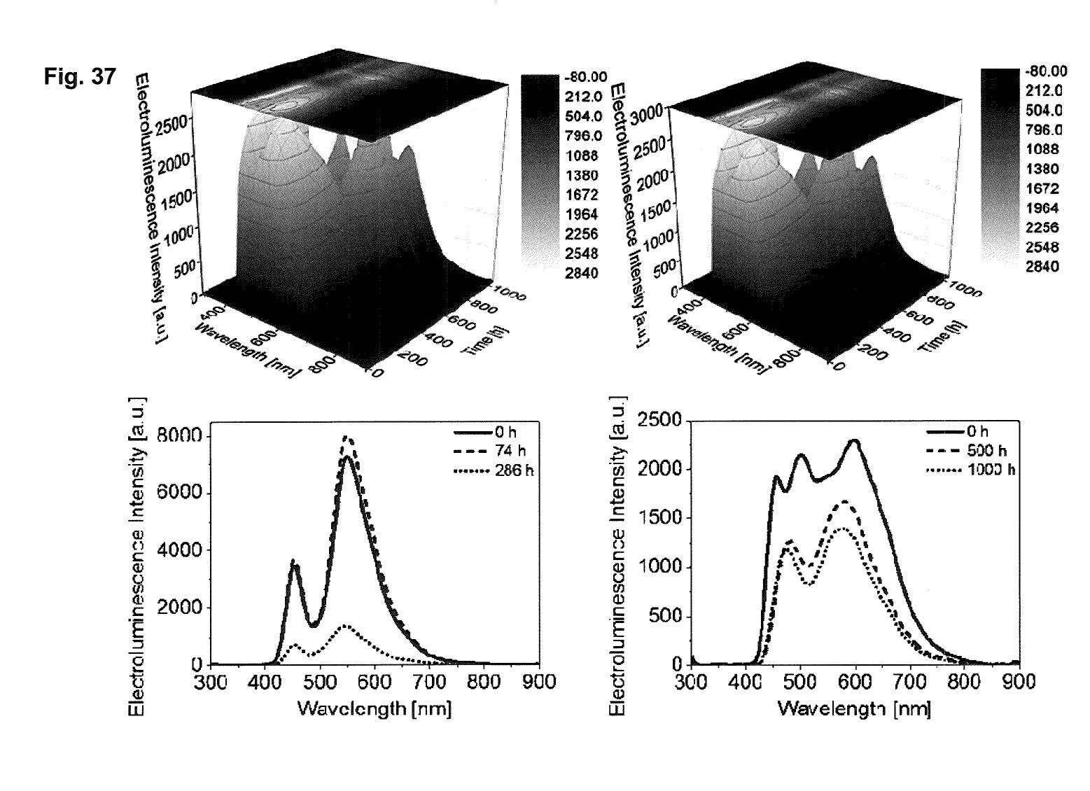

FIG. 37: Changes in the electroluminescence spectrum of P-WHLED blue-LED/5 (left) and CC-WHLED blue-LED/6/7 (right) over time. See Example 9.

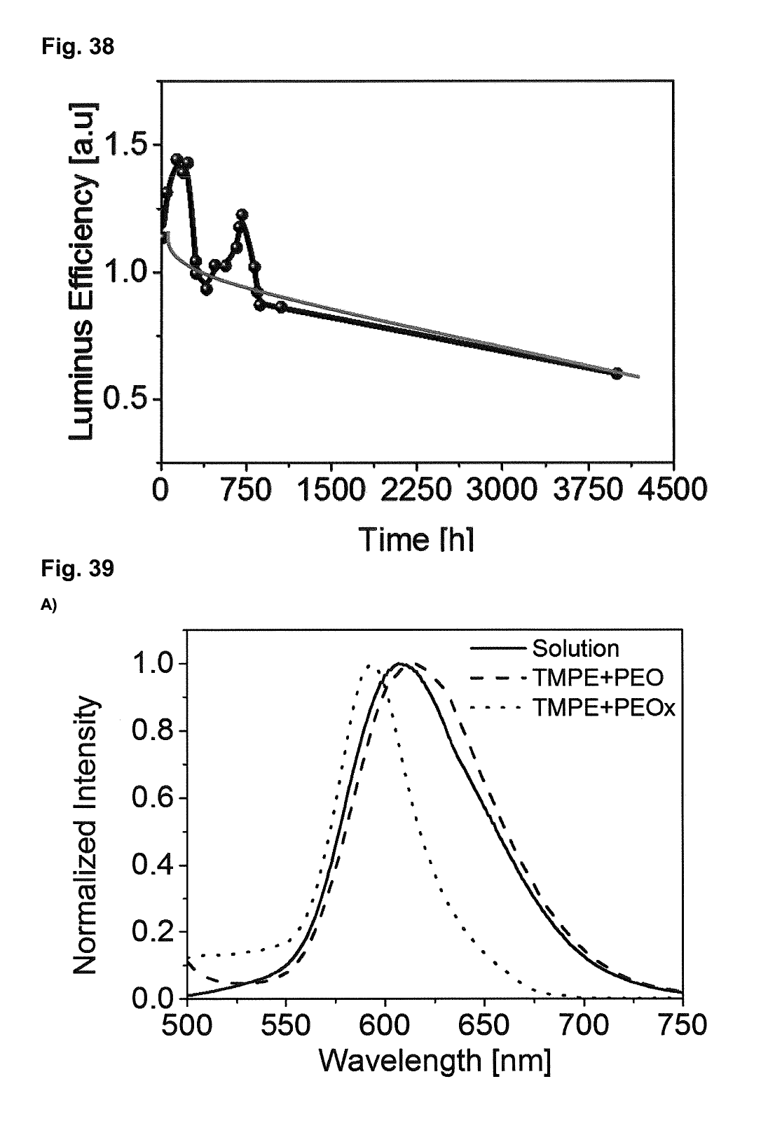

FIG. 38: Extrapolated lifespan of CC-WHLEDs. See Example 9.

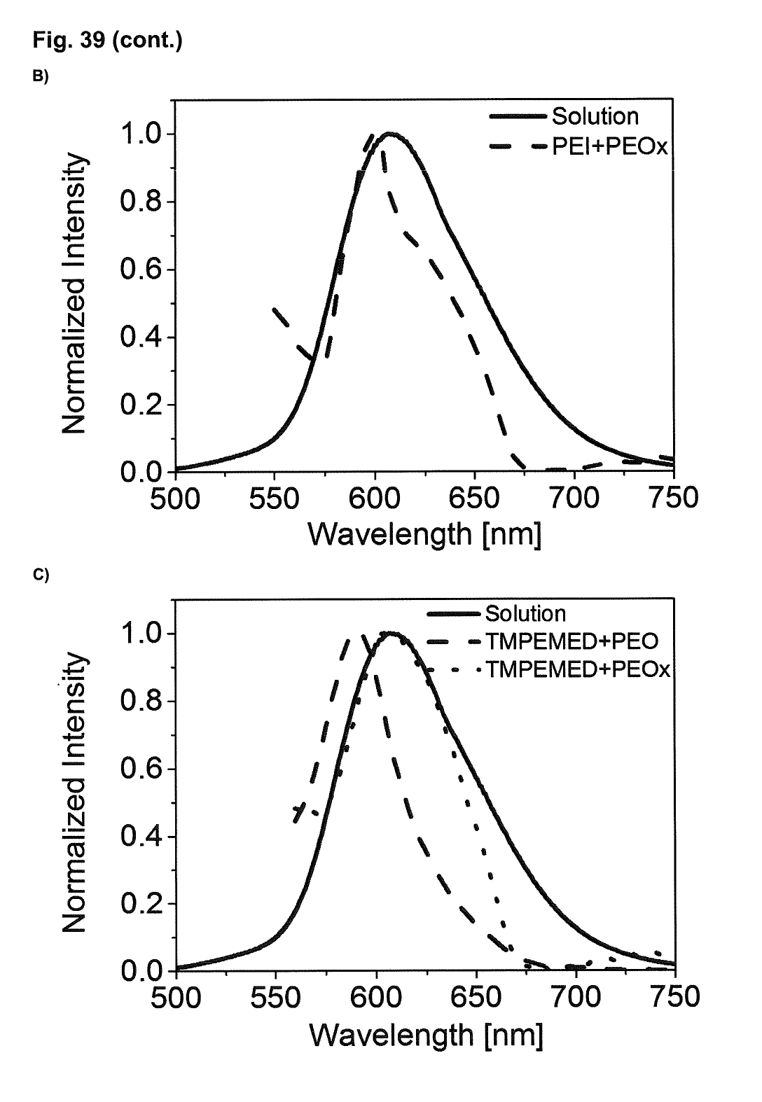

FIG. 39: Normalized photoluminescence spectra of the protein in solution and with different combinations of branched and linear polymers. See Examples 10 (A), 11 (B) and 12 (C).

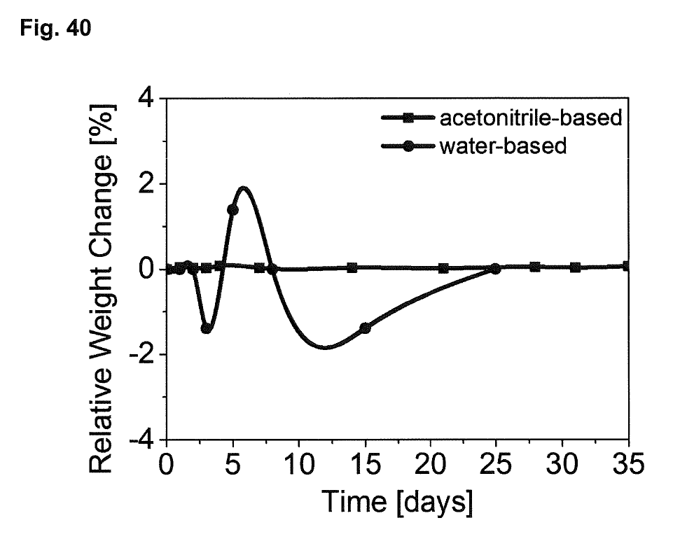

FIG. 40: Relative weight change of the water- and acetonitrile-based rubber-like films under storage conditions versus time. See Example 13.

The invention will now be described by reference to the following examples which are merely illustrative and are not to be construed as a limitation of the scope of the present invention.

### EXAMPLES

### Example 1

### Preparation of Rubber-Like Materials Containing Proteins Immobilized Therein

### Preparation of Proteins and Enzymes (Recombinant Protein Expression and Purification)

The preparation and characterization of several different luminescent proteins and enzymes was performed as shown in FIG. 1. E. coli strain M15 [pREP4] harboring the appropriate plasmids (pQE-9 expression constructs all containing an N-terminal 6xHis-tag coming from the pQE-9 expression vector, Qiagen) were grown at 28° C. in LB medium containing Amp (200 μg/ml) and Kan (100 μg/ml) antibiotics to an optical density of approximately 0.5 at 600 nm. Recombinant protein expression was induced with 1 mM isopropyl β-D-1-thiogalactopyranoside at 28° C. After 4 h of growing at 28° C., cells were harvested and frozen at −20° C. Frozen bacteria cells were thawed and lysed chemically using lysozyme and mechanically using a sonicator. Expressed proteins were then purified out of the cleared cell lysate using Ni-NTA affinity chromatography under native conditions, following the QIAGEN protocol (Henco K, A handbook for high-level expression and purification of 6xHis-tagged proteins—Third edition, 1991). The concentration of the resulting purified proteins were measured and the samples were subjected to further analysis (entrapment/integration into hydrogels). The steady-state absorption and photoluminescence features of the luminescent proteins in solution corroborate their successful preparation (see FIGS. 10 and 11).

In this example, various different fusion proteins (containing either a fluorescent protein or an enzyme, which is fused to a human adaptor domain such as SH2, SH3 or PABC) were used because they were readily available. In the case of luminescent or fluorescent proteins, the use of such fusion proteins is advantageous due to the increased molecular weight and an increase in stability. However, the corresponding proteins (not fused to any adaptor domain) can also be used and will give analogous results.

### Preparation of Rubber-Like Materials Containing Proteins Immobilized Therein

Before the formation of the rubber-like protein-based materials, a protein-based gel is formed. As a first step, the above-mentioned solutions with the different proteins are mixed with branched and linear poly(ethylene oxide) compounds—i.e., trimethylolpropane ethoxylate (TMPE) with Mₙ of 450 mol. wt. and linear poly(ethylene oxide) (I-PEO) with Mₙ of 5×10⁶ mol. wt., with a mass ratio of 4:1, respectively. The terminal hydroxyl groups provide a high compatibility with the protein solution, retaining enough water molecules within network. The gel network is mainly provided by the TMPE, while the I-PEO acts as a gelation agent (Prodanov L et al., Biomaterials 2010, 31, 7758). The mass ratio is optimized for the formation of a gel-like material with only the addition of an appropriate amount of water. In the studied range of protein concentrations, the formation of the gel and the final rubber material are independent of the protein amount. The optimized mixture of protein:TMPE:I-PEO in an approximate mass ratio of 1:36:3 is best described as an initial suspension that upon strong stirring over night becomes a gel, as also shown in FIG. 10. A direct comparison of the luminescent features of the gels with those of the initial solutions indicates that there is no drastic denaturation or degradation of the protein during the gel formation (see FIG. 10) (Prodanov L et al., Biomaterials 2010, 31, 7758); the small changes—i.e., maxima shift of around 5-10 nm and slight broadening of the spectrum—noted when comparing the emission features from solution, gel and rubber-like materials are produced by small conformational changes of the protein skeleton, which do not significantly affect the binding pocket of the chromophore; note that denaturation of the luminescent proteins implies a loss of the photoluminescence features. Further corroboration is provided by transmission electron microscopy (TEM) assays that show that the proteins are perfectly embedded in the gel network (see FIG. 2).

As a second step, the gel is deposited via doctor-blading onto any kind of substrate like, e.g., quartz (see FIG. 10). The doctor-blading was performed using a rectangular stamp of a thickness of 50 μm that was placed onto the support. Subsequently, the films were introduced into a vacuum station under 1-10 mbar for less than 1 h. The final layer is best described as rubber-like material in which the loss of a low percentage of water—i.e., ≤1.5% wt.—provokes the collapse of the network structure. Notably, the water is not recovered over weeks under ambient storage conditions (see FIG. 3). The rubber-like protein-based materials are easily pilled off from the substrate with tweezers and can be easily transferred to another substrate, as also shown in FIG. 10. As example, the color and composition of the films can be easily controlled by mixing in the protein solution different mass ratios of green and red fluorescent proteins. The thickness of the rubber-material can be controlled either by the thickness of the stamp or by the subsequent deposition of one layer on top of another with an excellent adhesion showing roughness lower than 10% (see FIG. 4).

### Detailed Procedure for Preparing Rubber-Like Materials Containing Fluorescent Proteins Immobilized Therein

The preparation of the above-discussed rubber-like materials containing a fluorescent protein immobilized therein (see FIG. 1) will be described in more detail in the following:

1.) Cloning of Recombinant Gene Constructs

To combine the different protein domains and to create the pQE-9 expression constructs the overlap-PCR method was performed. Using gene specific oligonucleotides eGFP was fused to the SH2-domain (eGFP), mCherry was fused to the SH3-domain (mCherry) and mTagBFP was fused to the PABC-domain (mTagBFP). Fluorescent proteins and protein interaction domains were separated by glycine-serine linker sequences allowing proper folding of both protein domains. The extension of the proteins results in larger and more stable fusion proteins. After PCR and gel extraction the DNA fragments were ligated into the pQE-9 E. coli expression vector that contains an N-terminal 6xHis affinity tag, using T4 DNA ligase. The right orientation of the constructs and the N-terminal in frame fusion with the 6xHis tag were guaranteed using specific restriction enzymes (see FIG. 1). After the ligation the recombinant plasmids were transformed into XL1 Blue E. coli cells and the correct sequence of the constructs was verified using Sanger Sequencing (GATC). For expression of the recombinant proteins pQE-9 plasmids, harboring the respective gene constructs, were transformed into E. coli M15 cells carrying the pREP4 repressor plasmid. Transformed E. coli cells were selected on plates containing ampicillin (pQE-9 expression vector, 200 μg/ml) and kanamycin (pREP4 repressor plasmid, 25 μg/ml).

2.) Preparation of Fluorescent Proteins E. coli strain M15 [pREP4] harboring the appropriate plasmids (pQE-9 expression constructs all containing a N-terminal 6xHis-tag coming from the pQE-9 expression vector, Qiagen) were grown at 28° C. in Lysogeny Broth (LB) medium (Bertani G, J Bacteriol. 1951, 62(3), 293-300) containing ampicillin (200 μg/ml) and kanamycin (25 μg/ml) antibiotics to an optical density of approximately 0.5 at 600 nm. Recombinant protein expression was induced with 1 mM isopropyl β-D-1-thiogalactopyranoside at 28° C. After 4 h of induction at 28° C., cells were harvested and frozen at −20° C. Frozen bacteria cells were thawed and lysed by lysozyme treatment and sonication. Recombinant proteins were affinity purified by Ni-NTA affinity chromatography under native conditions, according to instructions of the manufacturer (QIAGEN). The concentration of the resulting purified proteins were determined by measuring the absorption at 280 nm using a NanoDrop Spectrophotometer ND-1000 (Peqlab). The purified proteins are dissolved/stored in elution buffer (50 mM NaH₂PO₄, pH 8.0; 300 mM NaCl; 250 mM imidazole) until further use.

3.) Preparation and Characterization of the Protein-Based Gels and Rubber-Like Materials

The protein-based gels are prepared as follows. As a first step, the buffer solutions with the different proteins are mixed with a branched and linear poly(ethylene oxide) compounds—i.e., trimethylolpropane ethoxylate (TMPE) with Mₙ of 450 mol. wt. and linear poly(ethylene oxide) (I-PEO) with Mₙ of 5×10⁶ mol. wt. Both materials were purchased from Sigma Aldrich and used as received. The mass ratio is optimized for the formation of a gel-like material that allows the further film forming by using spin-coating or doctor-blading deposition techniques. In the studied range of the protein concentrations, the formation of the gels and the final rubber-like materials are independent of the protein amount. The optimized mixture of protein:TMPE:I-PEO in a mass ratio of 1:36:3 is best described as an initial suspension that upon strong stirring (1500 rpm) under ambient conditions over night becomes a gel (see FIG. 10). The presence of the fluorescent proteins were corroborated by steady-state spectroscopic techniques—steady-state absorption and photoluminescence characterizations were performed by using Perkin Elmer Lambda and Fluoromax-P-spectrometer from HORIBA Jobin Yvon, respectively. The refraction index was measured by using Krüss refractometer equipment from A Kross Optronic.

To prepare the rubber-like material, the gels are deposited via doctor-blading onto any kind of substrate like, for example, glass slides. The doctor-blading was performed using a rectangular stamp of a thickness of 50 μm that was placed onto the support. They can also be applied onto 3D substrates by introducing them into the gels. Subsequently, the films were introduced into a vacuum station under 1-10 mbar for less than 1 h. The final materials are best described as rubber-like material, which are easily pilled off from the substrate with tweezers and can be easily transferred to another substrate. The thickness of the rubber-material can be controlled either by the thickness of the stamp or by the subsequent deposition of one layer on top of another with an excellent adhesion. The thickness and roughness were measured using a profilometer DektakxT from Bruker.

### Example 2

### Preparation of Rubber-Like Materials Using Different Mass Ratios of Branched Polymer and Linear Polymer and Different Amounts of Aqueous Buffer Solution

The preparation of the gel and the rubber-like material according to the present invention is demonstrated using different mass ratios of the branched polymer (in this case, trimethylolpropane ethoxylate (TMPE) with an Mₙ of 450 Da) and the linear polymer (in this case, poly(ethylene oxide) (PEO) with an Mₙ of 5000 kDa), as shown in Tables 1 and 2 below, in the absence of a protein to be immobilized. The rubber formation is performed as described in Example 1.

In particular, the two polymers are mixed at different mass ratios as shown in Tables 1 and 2 below. Although TMPE is a low viscous liquid, the PEO does not dissolve even at high stirring conditions. To facilitate this process, several amounts of buffer solution (as otherwise used for the proteins) were added.

TABLE 1

| TMPE | PEO | Buffer | | Rubber |
|---|---|---|---|---|
| (mg) | (mg) | (μL) | Gel formation | formation |
| 60 | 5 | 50 | Highly viscous, not processable | yes |
| 60 | 5 | 100 | Highly viscous, but processable | yes |
| 60 | 5 | 150 | Good viscosity to make films | yes |
| 60 | 5 | 200 | Good viscosity to make films | yes |
| 60 | 5 | 300 | Low viscosity to make films | yes |
| 60 | 5 | 400 | Low viscosity to make films | yes |
| 60 | 5 | 500 | Low viscosity to make films | yes |

TABLE 2

| TMPE | PEO | Buffer | Gel | Rubber |
|---|---|---|---|---|
| (mg) | (mg) | (μL) | formation | formation |
| 60 | 1 | 150 | Low viscosity to | — |
| | | | make films | |
| 60 | 5 | 150 | Good viscosity to | yes |
| | | | make films | |
| 60 | 10 | 150 | Good viscosity to | yes |
| | | | make films | |
| 60 | 20 | 150 | Highly viscous, | yes |
| | | | not processable | |

As summarized in Table 1, the gel formation is good until 200 μL buffer, but only gels made with 150 μL and 200 μL are good enough to make films by using a doctor blading technique. The next step was to determine the lowest amount of PEO. As shown in Table 2, 5-10 mg of PEO is the best amount to obtain a useful gel. As a summary, the PEO:TMPE mass ratios of 1:6 and 1:12 with a certain amount of water (150 μL) were found to be the best conditions for further processing.

Further experimental results on gel formation and rubber-like material formation using various non-aqueous solvents are summarized in the following Table 3 (DMSO=dimethyl sulfoxide; DCB=dichlorobenzene; THF=tetrahydrofuran):

TABLE 3

| | | TMPE | PEO | Volume | Gel | Rubber |
|---|---|---|---|---|---|---|
| | | (mg) | (mg) | (μL) | formation | formation |
| polar protic | EtOH | 60 | 5 | 50 | too liquid, immiscible | |
| | | 60 | 5 | 150 | too liquid, immiscible | |
| | isopropanol | 60 | 5 | 50 | too liquid, immiscible | |
| | | 60 | 5 | 150 | too liquid, immiscible | |
| aprotic | acetonitrile | 60 | 5 | 50 | too liquid, immiscible | |
| | | 60 | 5 | 150 | good | possible |
| | DMSO | 60 | 5 | 50 | too liquid, immiscible | |
| | | 60 | 5 | 150 | too liquid, immiscible | |
| apolar | DCB | 60 | 5 | 50 | too liquid, immiscible | |
| | | 60 | 5 | 150 | too liquid, immiscible | |
| | THF | 60 | 5 | 50 | too liquid, immiscible | |
| | | 60 | 5 | 150 | too liquid, immiscible | |

### Example 3

### Application of the Rubber-Like Materials Containing Proteins Immobilized therein as Down-Converting Encapsulating Systems

FIG. 5 shows a sketch of a hybrid light-emitting diode with the rubber-like material containing a protein immobilized therein (see Example 1) as down-converting encapsulation system. A commercial blue emitting LED (purchased from Luxeon) with a electroluminescence spectrum at 450 nm was used in this example. To coat the 3D form of the previous silicone encapsulation, the LED can be either immersed into the gel for several seconds and/or the gel can be deposited by drop-casting onto the support surface. Subsequently, the coating is dried as described in Example 1 above. The device can be driven at constant and/or pulsed current and voltage schemes. In the example, the LED is driven at constant current of 10 mA using Keithley 2400 and the electroluminescence spectra and device performance were monitored with an integrating sphere (Avasphere 30-Irrad) coupled to an Avantes spectrophotometer (Avaspec-ULS2048L-USB2). The device was driven under ambient conditions.

### Example 4

### Application of the Rubber-Like Materials Containing Proteins Immobilized therein for Diagnostic Purposes

FIG. 6 shows a sketch of a diagnostic device based on the rubber-like material containing a protein immobilized therein (see Example 1). The rubber-like materials containing an enzyme immobilized therein were prepared onto glass substrate following the procedure described in Example 1 above. An aliquot of the reagent solution (containing NAD in a buffer composition)—i.e., 20 μl—was drop-casted onto the rubber-like material containing the enzyme immobilized therein. The drop was dried for several minutes under ambient conditions, allowing the immediate transformation from NAD to NADH. The blue fluorescence of the NADH was monitored under UV irradiation at 310 nm and 60 Watt.

### Example 5

### Storage Stability and Thermal Stability of the Rubber-Like Materials Containing Proteins Immobilized Therein

The stability of the rubber-like materials containing proteins immobilized therein (see Example 1) was investigated under ambient conditions—i.e., storage stability—and heating steps from room temperature to 90° C. with 10° C. steps each for 20 minutes in air—i.e., thermal stability. To this end, the absorption features of the two sets of experiments were monitored over time. As shown in FIGS. 7 and 12, the proteins in both gels and rubber materials exhibited a sound stability over several weeks under ambient storage conditions. Concerning the thermal stability, the absorption features do not change until temperatures of around 60-80° C., from which the absorption spectra is featureless due to the denaturation of the protein. These findings clearly demonstrate that the conformation of the proteins is preserved during the formation of both the gels and the rubber-like materials and even when they are stored under ambient conditions for several weeks. This is per se a remarkable result, since it is well known that proteins are prone to denature in solution under the above-mentioned conditions (Mozziconacci O et al., Adv Drug Deliv Rev. 2015, DOI: 10.1016/j.addr.2014.11.016; Davies M, Aust Biochem. 2012, 43, 8).

### Example 6

### Activity Measurements of Enzymes Immobilized in the Rubber-Like Material

FIG. 8 shows the basic principle of a diagnostic device using the rubber-like material containing a protein immobilized therein according to the invention. The enzyme activities (invertase and phosphoglucoisomerase) were measured using a modified coupled optical test based on monitoring the increase of NADH at its absorption maximum of 340 nm via a spectrophotometer. In these photometrical assays the turnover of sucrose/glucose is linked to an NADH producing reaction. Consequently, the increase of NADH is a measure of the amount of sucrose/glucose turnover in this reaction, indicating the enzymatic activity.

Invertase Assay

The buffer for the measurement contains the substrate (sucrose, 10 mM), ATP (2 mM), NAD (1 mM), Hepes/KOH pH 7.6 (100 mM), MgCl₂ (10 mM), hexokinase (1 U), phosphoglucoisomerase (PGI, 1 U, not shown) and glucose-6-phosphate dehydrogenase (G6P-DH, 1 U). When the invertase is active, it hydrolyzes sucrose into glucose and fructose. These hexoses can then be phosphorylated in an ATP-dependent manner by hexokinase. Glucose-6-phosphate is further oxidized by the enzyme glucose-6-phosphate dehydrogenase thereby producing NADH. As the absorption maximum of NADH and NAD⁺ differ, NADH can be exclusively detected at 340 nm. The pathway for fructose is not shown as it is the same as for glucose. The phospoglucoisomerase in the buffer converts fructose-6-phosphate into glucose-6-phosphate.

Hexokinase Assay

The buffer for the measurement contains the substrate (glucose, 5 mM), ATP (2 mM), NAD (1 mM), Hepes/KOH pH 7.6 (100 mM), MgCl₂ (10 mM) and glucose-6-phosphate dehydrogenase (G6P-DH, 1 U). If the hexokinase is active the substrate glucose is phosphorylated. Glucose-6-phosphate is converted into 6-phosphogluconolacton and NAD⁺ is reduced in parallel. As the absorption maximum of NADH and NAD⁺ differ, NADH can be exclusively detected at 340 nm.

Phosphoducoisomerase (PGI) Assay

The buffer for the measurement contains the substrate (fructose-6-phosphate, 5 mM), NAD (1 mM), Hepes/KOH pH 7.6 (100 mM), MgCl₂ (10 mM) and glucose-6-phosphate dehydrogenase (G6P-DH, 1 U). If the PGI is active, fructose-6-phosphate is isomerized into glucose-6-phosphate, which then can be oxidized in an NADH producing reaction. As the absorption maximum of NADH and NAD⁺ differ, NADH can be exclusively detected at 340 nm.

In the assays described above, the presence of the luminescent features of the NADH shows that the activity of the tested enzymes is retained when they are immobilized in a rubber-like material according to the present invention. This clearly indicates their possible application into detection kits for diagnostics.

### Example 7

### Application of the Rubber-Like Material Containing a Protein Immobilized therein in Hybrid White Light-Emitting Diodes (HLEDs)

In this example, a novel approach to fabricate bio-inspired hybrid white light-emitting diodes (white bio-HLEDs) combining UV- and blue-LEDs with a novel coating system using blue, green, and red fluorescent protein-based rubber materials according to the present invention is described. Three aspects constitute the main achievements of this work. Firstly, it has been demonstrated how fluorescent proteins can be used as novel down-converting materials, fulfilling the necessary requirements for this purpose, namely eco-friendly and low-cost production, easy color tunability with moderate fluorescence quantum yields, and large absorption extinction coefficients (Shcherbakova D M et al., Curr Opin Chem Biol. 2014, 20, 60; Chudakov D M et al., Physiol Rev. 2010, 90, 1103). The limitations are the need of aqueous buffer solutions, which prohibits standard coating techniques, and their moderate stability in solution under ambient conditions and/or moderate temperatures. Here, the second achievement sets in. To circumvent these problems, a new coating protocol has been developed that allows an easy-to-do homogenous covering of any kind of substrates, bringing fluorescent proteins closer to optoelectronic applications. This was possible by designing a sealing-free protein-based gel that transforms into a rubber-like material under moderate vacuum conditions. More importantly, the proteins embedded in both, gel and rubber materials, stay non-denatured for surprisingly long periods of time under ambient conditions. Thirdly, the major benefit of using a rubber material for encapsulation is the easy fabrication of a cascade architecture with a bottom-up energy transfer process (see FIG. 9), allowing a perfect covering of the whole visible spectra. These unique characteristics have led to the first white bio-HLED featuring 50 Lum/W with a loss of less than 10% after more than 100 h under operation conditions.

The blue (mTagBFP), green (eGFP), and red (mCherry) fluorescent proteins and corresponding gels were prepared as described in Example 1. It is postulated that the gel provides an excellent media in terms of rigidity and moisture to preserve the protein folding. The refraction index of the gels was also determined. Independently of the type of protein, all gels showed an average refractive index of 1.43-1.44. This value is close to the ideal one for encapsulation materials used in LEDs like silicone (Ma M et al., Opt express, 2011, 19,

A1135).

Although the gels show an excellent viscosity that allows the preparation of soft-films onto glass slides by means of doctor-blading technique, these films are not suitable for encapsulation purposes. However, the hardness of the films can be easily improved by partially drying them in a vacuum station, as described in Example 1. During this process, a water loss of around 1.5 wt. % is noted, leading to hard-films featuring mechanical properties that permit to describe them as rubber-like materials. For instance, the films are easily piled off from any substrate and even can be stretched and crumpled to obtain, for instance, a ball that keeps the luminescent features (see FIG. 10). As explained in Example 1, it is noteworthy that the water is not recovered after several weeks under ambient storage conditions (see FIG. 3), which indicates that no further encapsulation is necessary. Independently of the type of proteins, hard-films with a thickness up to a few millimeters with a low average roughness value are easily achieved by sequential repetition of doctor-blading and dryness processes (see FIGS. 4 and 10). In addition, the collapse of the network during the drying process increases the refractive index to values of at least 1.8, which is the detection limit of the apparatus used. This indicates that the Fresnel reflection loss should be further suppressed when the coating of the LED is performed with the final drying process (Ma M et al., Opt express, 2011, 19, A1135). Finally, the rubbers (like the corresponding gels) show an advantageous storage stability (see Example 5 and FIG. 12). In light of the aforementioned, the rubbers/rubber-like materials are highly suitable for down-conversion coating purposes compared to the gels.

Encouraged by these findings, white bio-HLEDs were fabricated combining UV- and blue-LEDs—maxima at 390 and 450 nm, respectively—with the coating system based on the preparation of blue, green, and red emitting protein-based rubbers. For reference purposes, the LEDs were firstly modified with coatings of different thicknesses lacking the proteins. As an example, the coated blue-LED was driven at different driving currents featuring no change in the electroluminescence (EL) spectra, while the luminous efficiency value is enhanced of around 20% with a coating thickness of up to 500 μm (see FIG. 13). This is related to the high refractive index of the rubbers that enhances the light collection, as the photopic sensitivity of the human eye is not affected in this experiment (Ma M et al., Opt express, 2011, 19, A1135).

Next, the UV- and blue-LEDs were modified with a single blue, green, and red protein-based coating. As expected from the excitation features of the fluorescent proteins (see FIG. 11), the down-conversion efficiency (η_{con}), which is defined as the ratio between the maxima of the LED and down-converting EL bands upon applying different currents, is excellent for the combinations UV-LED/mTagBFP and blue-LED/eGFP, as also shown in FIGS. 14 and 15. Even more striking, these devices feature η_{con} values that exceed the 100%, which is a requirement to efficiently further down-convert the emission of the fluorescent coating by applying another coating with a protein that absorbs the excess of emission of the bottom coating. In other words, the superior down-conversion features of the protein-coating allows to fabricate a cascade encapsulation featuring a bottom-up energy transfer process that provides an EL spectrum with the maxima peaks of each coating as shown in FIG. 14.

After a careful optimization, white bio-HLEDs with the architectures UV-LED/mTagBFP/eGFP/mCherry (referred to as “architecture 1” in the following) and Blue-LED/eGFP/mCherry (referred to as “architecture 2”) were successfully prepared and analyzed. In particular, upon increasing the driven current (see FIGS. 14 and 15), the EL spectra clearly shows the different maxima of the fluorescent proteins in concert with a η_{con} that remains over 100% until surprisingly high driving currents. At this driven regime, both bio-HLEDs feature an excellent white color stability in terms of color coordinates—0.35-0.35 (architecture 1), 0.32-0.33 (architecture 2), CRI—70-60 (architecture 1), 75-80 (architecture 2), and CCT—4500-6000 K for both architectures 1 and 2. Upon applying high driving currents, the LED emission slowly becomes dominant, as shown in FIGS. 14 and 15. This issue can be solved by increasing the protein content or increasing the thickness of the coating. Nevertheless, it is worth mentioning that the luminous efficiency of LEDs decreases upon applying high driven currents due to a reduction of the internal quantum efficiency. As an example, FIG. 16 shows how the luminous efficiency of architecture 2 maximizes up to applied currents of around 20 mA, from which this value exponentially decreases. As such, we decided to drive this device at 10 mA, monitoring the changes of the EL spectra and the luminous efficiency over time (see FIG. 16). The same experiment was performed with architecture 1 as shown in FIG. 17.

Besides the excellent color quality due to the shape of the EL spectrum, the stability of the bio-HLEDs is also sound. FIG. 16 clearly shows that the EL spectra remains almost constant showing a degradation of the top coating after 50-70 h under operation conditions. This is quite likely related to the oxidative stress caused by the formation of OH and/or peroxide radicals that oxidize and hence denature the proteins (Mozziconacci O et al., Adv Drug Deliv Rev. 2015, DOI: 10.1016/j.addr.2014.11.016; Davies M, Aust Biochem. 2012, 43, 8). More interesting is the change in the luminous efficiency, which features a decay lower than 10% with respect to the initial value after 100 hours. This value is remarkable compared to the current state-of-the-art HLEDs. Up to date, LEDs with a similar architecture to the bio-HLEDs provided in accordance with the present invention show a rapid degradation within a day, while a less than 10% loss in luminous efficiency over 100 hours is achieved if the down-converting coating is deposited onto a glass substrate, which is placed onto the LED with a separation of around 5 mm (Findlay N J et al., Adv Mater. 2014, 26, 7290).

In conclusion, the present invention provides the first bio-HLEDs featuring a protein-based cascade coating, which allows a perfect covering of the whole visible spectrum with a loss of less than 10% in luminous efficiency over 100 h. This has been achieved by developing a new technique to stabilize and to process fluorescent proteins in a gel that after a drying process under gentle vacuum conditions becomes a rubber material that is suitable for coating purposes. Here, it has been demonstrated that the synergy of the excellent features of the fluorescent proteins—i.e., excellent storage stability and complementary absorption-emission features—with the easy processability of the rubber material can be exploited for designing a cascade coating suitable for lighting applications. This is the first example in which a cascade coating has been applied into HLEDs. Overall, this work opens up a new route to exploit fluorescent proteins in optoelectronic applications, and particularly in lighting applications with HLEDs.

Further details on the fabrication and characterization of the above-described bio-HLEDs are provided in the following: The UV- and blue-LED were purchased from Roschwege GmbH and Luxeon, respectively. The preparation of the bio-HLEDs concerns a two steps procedure. Firstly, the proteins-based gels (see Example 1) are deposited onto the LED wetting completely the surface. Secondly, the coated LED is transferred to the vacuum chamber under 1-10 mbar for less than 1 h. This procedure is repeated to enhance the light down-conversion efficiency of the HLED. As well, the design of a cascade coating is easily performed by repeating the above-described steps depositing subsequently blue, green, and red gels. Independently of the thickness of the coating, it can be easily piled off from the LED surface for a further analysis. The optimized thickness of the coating for devices with the architectures 1 and 2 is close to 1-1.5 mm, respectively. The bio-HLEDs were characterized by using a Keithley 2400 as a current source, while the luminous efficiency and changes of the electroluminescence spectrum were monitored by using Avantes spectrophotometer (Avaspec-ULS2048L-USB2) in conjunction with a sphere Avasphere 30-Irrad.

### Example 8

### Application of the Rubber-Like Material Containing a Protein Immobilized therein in a Bioreactor

The general concept is to transform a reactant into the desired product by passing the reactant solution through the rubber-like material, which contains an enzyme as the active component, by means of a vacuum system, as illustrated in FIG. 18.

The example was performed with phosphoglucoisomerase (PGI) enzyme, the reactant NAD (featureless emission), and the product NADH (blue emitting material). Since the rubber-like material gets dissolves into the reactant solution within 5-10 min, a moderate vacuum of 100-150 mbar needs to be applied and a low amount of the reactant solution is used. Secondly, a strong vacuum of around 10-30 mbar is applied to dry and then recover the rubber-like material. Using this procedure, around 20 mL of the reactant solution has been converted into the product.

This example demonstrates that the rubber-like material according to the invention, containing an enzyme immobilized therein, can advantageously be used in a bioreactor.

### Example 9

### Preparation and Applications of Rubber-Like Materials Containing Non-Protein Active Materials Immobilized Therein

In this example, an easy-to-do protocol for preparing luminescent rubber-like materials based on a wide palette of active compounds, such as small-molecules, quantum dots, polymers, and coordination complexes is exemplified. The combination of this protocol with that for preparing similar rubbers based on fluorescent proteins states the universal character of this approach. This is further assessed by using comprehensive spectroscopic and rheological investigations. Furthermore, the novel luminescent rubbers are applied as down-converting packing systems to develop white hybrid light-emitting diodes (WHLEDs), which are heralded as a solid alternative to achieve energy-saving, solid-state, and white-emitting sources. As such, this work also provides a clear prospect of this emerging lighting technology by means of a direct comparison among WHLEDs fabricated with all the above-mentioned down-converting systems. Here, the use of rubbers based on coordination complexes outperforms the others in terms of both luminous efficiency and color quality with an unprecedented stability superior to 1,000 h under continuous operation conditions. This represents an order of magnitude enhancement compared to the state-of-the-art WHLEDs, while keeping luminous efficiencies of around 100 Im/W.

Introduction

The development of efficient and stable white solid-state lighting sources is one of the key technological research forefronts, as incandescent light bulbs and fluorescent lamps have reached their limit in terms of balancing luminous efficiency, stability, and environmental/recycling issues.¹ Two main alternatives are almost ready-to-go towards the next generation of sustainable bulbs. On one hand, organic light-emitting diodes (OLEDs) are a potential technology to provide flexible and thin lighting sources for screens and in-door luminaires.² However, despite efforts during the last 20 years, white OLEDs still show a clear trade-off in terms of low-cost production and high performance.² On the other hand, inorganic white light-emitting diodes (WLEDs) have been strongly developed by both industry and scientific communities since the pioneering works on blue-LEDs by Akasaki, Amano, and Nakamura et al. in the early 90′s.³ Generally speaking, the chip of the blue- or UV-emitting LEDs is coated with inorganic down-converting phosphors based on rare-earth elements like the archetypal Y₃Al₅O₁₂; YAG:Ce and its derivatives.⁴ As a result, the combination of the LED emission and that of the down-converting coating leads to WLEDs featuring high luminous efficiencies and stabilities when the packing system is optimized. Their main drawbacks are i) the high production cost due to the use of rare Earth crust materials, ii) the scarcity of efficient deep-red emitters that compromises the color quality of the white emission, and iii) the lack of efficient protocols to recycle these materials. Thus, this strategy barely addresses the basis of green economics in terms of ecological sustainability in concert with a low-cost production.¹

As an alternative, recent research has explored the possibility of using eco-friendly organic down-converting materials in the so-called white hybrid inorganic/organic LEDs (WHLEDs).⁵⁻⁸ Similar to WLEDs, the architecture of WHLEDs consists of a standard inorganic blue- or UV-LED, in which the encapsulation system is replaced by an organic-based down-converting material, which upon continuous excitation features a broad low-energy emission band (see FIGS. 19 and 20). This architecture has recently led to WHLEDs with high color quality—i.e., commission international de I'Éclairage (CIE) coordinates of 0.30-3/0.30-3, color rendering index (CRI) above 90, and correlate color temperature (CCT) between 2,500-6,500 K, but still with low stabilities of around 100 h due to either degradation of the luminescent material, of the matrix, or both upon continuous excitation under ambient conditions.⁵⁻⁸

Up to date, there are four different approaches to develop down-converting coatings. Firstly, thin films, which consist of a mixture of organic materials with UV- or thermal-curable sealing reagents, are typically deposited either onto a glass substrate or on top of the packing of the LED (see FIG. 20).⁵ Several authors have shown that both the degradation of the down-converting materials under continuous excitation and a phase separation in the morphology of the coating upon preparation are common factors that limit the stability of the WHLEDs up to values of a few hours.^{5i,k,l} However, strategies based on pre-encapsulating the luminescent material or increasing the gap between the LED and the down-converting coating provide stabilities of approximately hundred hours.^{5d,o,q} Secondly, an interesting alternative approach was reported by Li and Su et al. in 2013.⁶a The authors proposed the use of metal organic frameworks (MOFs) that show high-energy emission features and pores of tunable sizes, in which one or a mixture of several d own-converting materials can be embedded (see FIG. 20).⁶ As such, the inorganic LED excites either both the MOF and the adsorbed organic moiety or only the MOF that further transfers the energy to the organic moiety. More interesting, several groups have started to show that the MOF-based approach is compatible with quantum dots, coordination complexes, and small-molecules.⁶ Thus, it bears a great potential for commercial purposes. As state-of-the-art WHLEDs with the MOF encapsulation, white devices featuring CRI from 70 to 90 and luminous efficiency beyond 50 Im/W have been achieved, but still their stability has not been studied in depth. Thirdly, a new coating based on a cellulose derivative (see FIG. 20), in which, for example, inorganic and graphitic quantum dots have been embedded, has led to new WHLEDs spanning the whole visible spectrum.' Here, the devices have shown CIE coordinates of 0.33/0.37 and efficiency values up to 31.6 Im/W, but the stability has not been reported yet. Lastly, a new down-converting coating method based on the mixture of fluorescent proteins with a combination of branched and linear polymers in water has been developed. The latter form a gel that is further transformed into a luminescent rubber-like material that is easily applied as a packing system to fabricate bio-WHLEDs.⁸ Similar CRI and luminous efficiencies to those noted for the other approaches have been shown, along with an encouraging stability of less than 10% loss of efficiency after 120 h. Here, the instability of the bio-WHLED was solely attributed to the degradation of the red-emitting proteins. In this example, it is demonstrated that the new rubber-like encapsulation method according to the invention can be easily modified to implement a wide variety of down-converting materials that span small-molecules, quantum dots, polymers, and coordination complexes. This is further supported by spectroscopic studies to determine the changes of the photoluminescence features of the down-converting materials embedded in the rubbers and by rheological assays to elucidate the mechanical properties of both the gels and rubbers. Finally, this work provides a roadmap for further implementations and developments, since a direct comparison between WHLEDs based on the above-mentioned rubbers is provided.

As the most remarkable result, the use of coordination complexes stands up among the others, featuring unprecedented stabilities of more than 1,000 h with a slight loss of luminous efficiency and no color degradation. The latter is further extrapolated to around 4,000 h, representing more than one order of magnitude enhancement in stability compared to the state-of-the-art WHLEDs. Hence, it is concluded that the combination of high luminous efficiency (100 Im/W) and stabilities of thousands of hours highlight the versatility and potentiality of the approach according to the invention for the development of WHLEDs for low- and mid-power applications.

Materials and Methods

1. Preparation and Characterization of the Gel- and Rubber-Like Materials

All the luminescent compounds, such as small-molecules, polymers, and coordination complexes were purchased from Merck and Sigma Aldrich and used as received. The carbon quantum dots were prepared according to literature.¹⁰ The gels were prepared as follows. As a first step, the branched and linear poly(ethylene oxide) compounds—i.e., trimethylolpropane ethoxylate (TMPE) with Mn. of 450 mol. wt. and linear poly(ethylene oxide) (l-PEO) with Mn. of 5×10⁶ mol. wt. and 1 mg of the luminescent compounds were mixed with different amounts of solvent—i.e., water or acetonitrile. Upon strong stirring (750 or 1500 rpm) under ambient conditions over night, this mixture becomes a gel. The mass ratio is optimized for the formation of a gel-like material that allows the further film forming via doctor-blading onto any kind of substrate like, for example, glass slides. The doctor-blading was performed using a rectangular stamp of a thickness of 50 μm that was placed onto the support. They can also be applied onto 3D substrates by introducing them into the gels. Subsequently, the films or coated materials were introduced into a vacuum station under 1-10 mbar for less than 1 h. The final materials are best described as rubbers, which are easily peeled off from the substrate with a tweezer and can be transferred to another substrate. The thickness of the rubbers can be controlled either by the thickness of the stamp or by the subsequent deposition of one layer on top of another with an excellent adhesion. The thickness and roughness were measured using a profilometer Dektak XT from Bruker. The presence of the luminescent materials was corroborated by spectroscopic techniques—steady-state absorption and photoluminescence characterizations, as well as excited-state lifetimes and photoluminescence quantum yields that were performed by using Perkin Elmer Lambda, Fluoromax-P-spectrometer (Horiba-JobinYvon), and SPEX Fluorolog-3 (Horiba-JobinYvon) supplied with an integrated TCSPC software. The refraction index was measured by using Krüss refractometer equipment from A Kross Optronic.

The rheological measurements of the gels and of the bare rubbers were carried out with an MCR 301 rheometer from Anton Paar at a temperature of 295.16 K. The gels were studied using a cone-and-plate geometry with a diameter of 25 mm and a cone angle of 1°. The oscillatory measurements of the rubbers were performed with a parallel-disk configuration with a plate diameter of 25 mm and a gap width of 1 mm. Amplitude sweeps were carried out at an angular frequency of 1 rad/s in a deformation ranged between 0.1% and 2% to determine the linear viscoelastic regime of the materials studied. Frequency sweeps were carried out in the linear viscoelastic regime at angular frequencies ranging from 0.1 to 100 rad/s. The study of the impact of luminescent materials on the rheological properties of the rubbers was performed with a narrow-gap rheometer in the parallel-disk configuration at a temperature of 297.76 K. It is based on a UDS 200 rotational rheometer from Physica. As disks, it uses glass plates of 75 mm and 50 mm diameter with an evenness of λ/4 and λ/10, respectively. The gap width is set up and measured independently from the rheometer with a confocal interferometric sensor resulting in a gap width with a precision of up to ±0.7 μm. Further details about this setup and its alignment are provided in H. Dakhil et al., Appl. Rheol. 2014, 24, 63795. The samples were squeezed at normal forces of about 5-9 N to a gap width of 200 μm.

2. Fabrication and characterization of the WHLEDs The blue-LEDs were purchased from Luxeon (LXHL-PRO3) and Winger (WEPRB3-S1). The preparation of the WHLEDs concerns a two-step procedure. Firstly, the gels are deposited onto the LED wetting the complete surface. Secondly, the coated LED is transferred to the vacuum chamber under 1-10 mbar for less than 1 h. This procedure is repeated to enhance the light down-conversion efficiency of the WHLED. As well, the design of a cascade coating is easily performed by repeating the above-described steps depositing subsequently high- and low-energy emitting gels. Independently of the thickness of the coating, it can be easily peeled off from the LED surface for a further analysis. The optimized thickness of the coatings is mentioned further below. The WHLEDs were characterized by using a Keithley 2400 as a current source, while the luminous efficiency and changes of the electroluminescence spectrum were monitored by using Avantes spectrophotometer (Avaspec-ULS2048L-USB2) in conjunction with a sphere Avasphere 30-Irrad.

Results and Discussion

As previously reported for bio-WHLEDs, the composition of the matrix—i.e., as shown in FIG. 20, branched and linear poly(ethylene oxide) compounds (b- and I-PEO, respectively) in different mass ratios—was optimized to form gels and rubber materials after mixing them with fluorescent proteins diluted in an aqueous media.⁸ Without the addition of water, neither the gel nor the rubber are formed. This could limit the versatility of this concept, as only compounds soluble in water could be applied. To challenge this statement, several solvents ranging from polar protic, to polar aprotic, and to nonpolar were used for the preparation of both gels and rubbers. Here, the gels were formed by mixing b- and I- PEO with different amounts of solvents. Upon strong stirring (750 or 1500 rpm) under ambient conditions over night, this mixture becomes a gel. The mass ratio is optimized for the formation of a gel-like material that allows the further film forming via doctor-blading onto any kind of substrate like, for example, glass slides. Independently of the amount of solvent employed, the composition of the b- and I-PEO mixtures, and the stirring conditions, only acetonitrile and water turned out to be suitable for forming homogenous gels under the conditions used in this example (see Table 4 and FIG. 21). Similar to water-based gels,⁸ the viscous properties of the acetonitrile-based gels allow an excellent handling for coating purposes. Indeed, the viscosity can be controlled by modifying the amount of the I-PEO (see FIG. 22). The mixture b-PEO:I-PEO=6:1 wt. with 150 μL of acetonitrile was chosen for the preparation of soft-films onto glass slides by means of a doctor-blading technique. Upon a drying process—i.e., a solvent loss of <1 wt. %—under gentle vacuum conditions the soft-films transform into a rubber material (as described above). The final materials are best described as rubbers, which are easily peeled off from the substrate with a tweezer and can be transferred to another substrate. Thicknesses of up to the millimetre regime with a low average roughness value (<10%) are easily achieved by sequential repetition of doctor-blading and drying processes (see FIG. 23). Rheology assays show that both water- and acetonitrile-based rubbers feature similar values for the storage (G′) and loss (G″) moduli, which quantify the elastic and the viscous material behaviour, respectively. The only exception are the rubbers with the highest I-PEO content (3:1 wt.), where the water-based rubbers show a higher mechanical stability than the acetonitrile-based ones (see FIG. 24).

TABLE 4
Test of the formation of the gel and rubber materials by changing different
parameters like the nature of the solvents, the amount of the solvents,
the b-PEO:I-PEO mass ratio, and the stirring conditions.

| | | Volume | b-PEO/I-PEO | Stirring | Gel | Rubber |
|---|---|---|---|---|---|---|
| | Solvent | [μL] | [wt.] | [rpm] | formation | formation |
| polar protic | Water | 50 | 6:1/12:1 | 750/1500 | Highly viscous | Yes |
| | | 150 | 6:1/12:1 | 750/1500 | Good viscosity | Yes |
| | Ethanol | 50 | 6:1/12:1 | 750/1500 | Immiscible | No |
| | | 150 | 6:1/12:1 | 750/1500 | Immiscible | No |
| | Isopropanol | 50 | 6:1/12:1 | 750/1500 | Immiscible | No |
| | | 150 | 6:1/12:1 | 750/1500 | Immiscible | No |
| polar aprotic | Acetonitrile | 50 | 6:1/12:1 | 750/1500 | Low viscosity | No |
| | | 150 | 6:1/12:1 | 750/1500 | Good viscosity | Yes |
| | Cyclohexanone | 50 | 6:1/12:1 | 750/1500 | Immiscible | No |
| | | 150 | 6:1/12:1 | 750/1500 | Immiscible | No |
| | THF | 50 | 6:1/12:1 | 750/1500 | Immiscible | No |
| | | 150 | 6:1/12:1 | 750/1500 | Immiscible | No |
| apolar | Toluene | 50 | 6:1/12:1 | 750/1500 | Immiscible | No |
| | | 150 | 6:1/12:1 | 750/1500 | Immiscible | No |
| | Hexane | 50 | 6:1/12:1 | 750/1500 | Immiscible | No |
| | | 150 | 6:1/12:1 | 750/1500 | Immiscible | No |
| | Chloroform | 50 | 6:1/12:1 | 750/1500 | Immiscible | No |
| | | 150 | 6:1/12:1 | 750/1500 | Immiscible | No |

The linear viscoelastic regime of both rubbers is restricted to less than 1% strain. Here, G′ is always larger than G″, but of the same order of magnitude. Frequency sweeps in the linear viscoelastic range revealed only a small frequency dependence, as it is typical for rubber-like materials. In general, the increase of the I-PEO content leads to an enhancement of G′ and G″ values (see FIG. 24). Finally, the refractive index of both rubbers was superior to 1.8, which is close to the ideal one for encapsulation materials used in LEDs like silicone.⁹ Thus, the new acetonitrile-based rubber is also suitable for encapsulation purposes in the preparation of WHLEDs.

Next, the possibility was exploited to use both water and acetonitrile solvents to integrate a wide palette of luminescent materials into the rubbers. To this end, commercial luminescent materials with different emission wavelength (λₑₘ) were selected, as described above, namely i) small-molecules like Coumarin 334 (1, λₑₘ=496 nm), Fluorescein 27 (2, λₑₘ=544 nm), zinc-tetraphenylporphyrin (ZnTPP) (3, λₑₘ=609, 624, 652 nm), ii) water soluble yellowish orange emitting carbon-based quantum dots (4, λₑₘ=450 nm (λ_{exc}=310 nm); λₑₘ=519 nm (λ_{exc}=390 nm); 549 nm (λ_{exc}=450 nm ,¹⁰ iii) an emitting polymer like Super Yellow (5, λₑₘ=550 nm), and iv) coordination complexes such as [Ir(ppy)₂(acac)] (6, λₑₘ=470, 490 nm) and [Ir(ppy)₂(tb-bpy)][PF₆] (⁷, λem=570 nm) where ppy is 2-phenylpyridine, acac is acetylacetone, and tb-bpy is 4,4-di-tert-butyl-2,2-dipyridyl. Their chemical structures are shown in FIGS. 25 and 26.

The gels were formed by mixing b-PEO:I-PEO in a ratio of 6:1 wt. and 1 mg of each luminescent materials with 150 μL of acetonitrile for 1-3/5-7 and water for 4. FIGS. 25 and 27 display the comparison of the normalized absorption and emission spectra in both solutions and rubbers, indicating that, in general, there is no degradation of the compounds upon rubber formation. However, a strong interaction between the small-molecules and graphitic quantum dots with the matrix in the rubber is highlighted by the red shifted (5-15 nm) absorption and emission spectra, as well as the decrease of the excited-state lifetimes, pointing to a quenching of the emission (see FIG. 27 and Table 5). On the contrary, the encapsulation of polymers and coordination complexes into the rubber matrix increases their excited-state lifetimes in minor and major fashions, respectively (see Table 5). This is quite likely related to the effective encapsulation of compounds into the matrix preventing the well-known emission quenching of ambient oxygen. As such, the matrix seems to be more suited for the luminescent polymers and coordination complexes. The addition of the luminescent materials does not have a major impact on both the formation and characteristics of the rubber materials in terms of the refractive index (>1.8) and the rheological parameters—i.e., G′ and G″ as shown in FIG. 28. Finally, the stability of the rubbers was investigated by monitoring the absorption features of the organic compounds under different scenarios, such as room conditions—i.e., storage stability, upon irradiation with a UV lamp (310 nm, 8 W) under ambient conditions—i.e., irradiation stability, and under a heating ramp ranging from room temperature to 120° C. with 20° C. steps under ambient conditions—i.e., thermal stability. As shown in FIGS. 29, 30, and 31, all rubbers show excellent storage stabilities over 40 days, while the irradiation stability is also sound for the rubbers based on the coordination complexes and quantum dots, but not for those containing small-molecules and polymers, which degrade after a few hours. Moreover, the changes in the absorption features upon heating clearly indicate that all rubbers are stable up to temperatures of 100° C.

TABLE 5
Photophysical properties of 1-7 in solution and rubbers.

| | |
|---|---|
| | Lifetimes_{sol/rubber} |
| PLQY_{sol/rubber} | [ns] |

| Compound | [%] | τ₁ | τ₂ |
|---|---|---|---|
| 1 | 68/30 | 0.70/0.48 | 3.11/1.11 |
| 2 | 87/36 | 0.97/0.24 | — |
| 3 | 3.3/1.1 | 1.28/0.32 | 1.95/1.05 |
| 4 | 20/5 | 2.60/1.64 | 10.30/8.27 |
| 5 | 69/74 | 0.70/1.0 | 0.22/0.27 |
| 6 | 3.9/15.7 | 103/651 | — |
| 7 | 7/35 | 65/383 | — |

Finally, a direct comparison of the stability of the luminescent compounds between the solutions and the rubbers under UV irradiation is shown in FIG. 32, indicating that the matrix further stabilizes all the compounds. This is clearly noted for the small molecules and polymers, while especially the carbon-based quantum dots and the coordination complexes show a sound irradiation stability in both solutions and rubbers. Hence, the interaction between the rubber components and the down-converting materials is beneficial in terms of stability and photophysical features.

Taking these findings into account, we fabricated white-emitting HLEDs combining a blue-LED—i.e., maximum 450 nm—with a cascade coating system combining rubbers with small-molecules (SM-WHLEDs), quantum dots (QDs-WHLEDs), polymers (P-WHLEDs), and coordination complexes (CC-WHLEDs), as described in the materials and methods section above. The preparation of the WHLEDs concerns a two-step procedure. Firstly, the gels are deposited onto the LED wetting the complete surface. Secondly, the coated LED is transferred to the vacuum chamber under 1-10 mbar for less than 1 h. This procedure is repeated to enhance the light down-conversion efficiency of the WHLED. Independently of the thickness of the coating, it can be easily peeled off from the LED surface for a further analysis. Noteworthy, the optimization of the thickness of the luminescent down-converting coatings was realized to obtain the right balance between white color quality and high luminous efficiency as shown in

FIG. 33 and discussed below. As starting conditions, the performance of the WHLEDs was measured under dried N₂ atmosphere and the most stable devices were afterwards measured under ambient conditions.

SM-WHLEDs feature an architecture of a blue-LED with a top coating based on 1 (0.14 mm)/2 (0.06 mm)/3 (0.10 mm). Upon increasing the driving current from 10 to 250 mA, the electroluminescence spectra clearly show distinguishable peaks for all small-molecules with a stable white color with, for example, CIE coordinates of 0.35/0.35 to 0.28/0.28, CRI values of 93 and 78, and CCT of 4,776 and 10,851 K for 10 and 250 mA, respectively (see FIG. 34). Importantly, this is independent of the visual angle (0, 30, 45, 90°) as the coating homogenously covers the whole packing (see FIG. 34). Next, the long-term stability of the device was studied under a driving current of 10 mA. As shown in FIGS. 34 and 35, even at this mild operation condition and under inert atmosphere, the initial white light with CIE coordinates of 0.31/0.32 changes in a few hours toward the blue region with CIE of 0.23/0.16. Moreover, the CRI decreases from 77 to 64 and the CCT decreases from 6,600 to 5,900 K. Finally, the luminous efficiency significantly reduces due to changes in the emission spectrum—i.e., ca. 1 h with a loss >30%. This result is expected as the small-molecules show a sound photo-assisted degradation in both solutions and rubbers (see FIGS. 30 and 32). Thus, no further experiments were performed with this family of luminescent compounds.

Not being able to obtain stable white lighting sources with small-molecules, we turned to investigate the QDs-WHLEDs with blue-LED/4 (0.1 mm). Similar to the SM-WHLEDs, white devices were easily achieved independently of the applied driving currents (see FIG. 34). More interesting, the quality of the white color was monitored over time under operation conditions and inert atmosphere, showing CIE coordinates of 0.33-2/0.32-0, CRI value of ˜90-95, and a CCT of 5,500-6,200 K for a time of ca. 20 h (see FIGS. 34 and 35). Beyond this operation time, the red contribution of the electroluminescence spectrum gets more prominent, changing the white quality with CIE coordinates of 0.36/0.32, CRI values of 90, and CCT values of 4,100 K, as well as slightly reducing the luminous efficiency.

Although the changes of the photoluminescence behavior of this type of materials under different excitations and environmental conditions—i.e., temperature, pH, irradiation, etc.—is still under debate,¹⁰ this might be related either to interactions of the outer substituents with matrix that promote emission from trapping states or to a release of the peripheral substituents changing the core of the QDs. At this point, the QD-WHLED was probed under ambient conditions.

During the first 30 h, the electroluminescence spectra quickly evolved until a more balanced contribution in the yellow and red parts (see FIGS. 34 and 35), but with a more prominent blue component—i.e., CIE of 0.33/0.32, CRI of 94, and CCT of 5,800 K. This also affects the luminous efficiency that further reduces (see FIG. 34). After this point, the electroluminescence spectrum is constant. Thus, there are two downsides of using this type of material, namely the initial low luminous efficiency of around 2 Im/W that is related to the poor photoluminescence quantum yields and the changes of the electroluminescence spectrum over long periods of time. It is important to note that a proper design of graphitic quantum dots with, for example, organosilane outer substituents and/or encapsulated carbon QDs might solve both problems as very promising results have been recently shown.^{5q}

Next, the use of well-known emitting polymers was investigated for the development of P-WHLEDs. The optimized device was a blue-LED/5 (0.1 mm), which independently of the applied current shows a broad electroluminescence spectrum with two maxima at 450 and 560 nm, corresponding to the blue-LED and the polymer, respectively (see FIG. 36). The quality of the white light is highlighted by almost no change in CIE coordinates from 0.32/0.34 along with CRI values slightly superior to 70 with a CCT of 6,150-5,900 K. The moderate CRI is attributed to a low electroluminescence intensity in the red region of the visible spectrum. More striking is the high luminous efficiency of around 200 Im/W that is stable over about 200 h under inert atmosphere. The efficiency value is similar to the best all-inorganic white-emitting LEDs. Thus, the same P-WHLED was subsequently probed under ambient conditions (see FIGS. 36 and 33). Unfortunately, the emission of the polymer is immediately damaged by the well-known photo-assisted oxidation process,^{5d, 11} as both the color quality and the luminous efficiency declined (see FIGS. 36 and 37). This finding is not surprising, since thin-film lighting devices based on this luminescent polymer have demonstrated stabilities of thousands of hours under inert atmosphere, but need of a rigorous encapsulation when it is tested out of the glove-box.^{11b,c} Indeed, the irradiation stability of 5 is also moderate in solution and rubbers compared to the other luminescent compounds (see FIGS. 30 and 32). Thus, a further encapsulation system will be necessary for improving the lifespan of the P-WHLEDs with the shortcoming of a less user-friendly fabrication process.

Finally, the CC-WHLEDs with the optimized architecture blue-LED/6 (0.1 mm)/7 (0.1 mm) were probed (see FIGS. 36 and 37). Upon applying different driving currents from 10 to 200 mA, clearly distinguishable emission peaks for the blue-LED, 6, and 7 were observed. As expected, CIE coordinates of 0.33-0/0.32-0, CRI values superior to 80, and CCT of 6,000-8,500 K were achieved. For comparison, the stability study of the CC-WHLEDs was carried out monitoring the changes of the electroluminescence spectrum and the luminous efficiency over time under inert atmosphere (see FIGS. 36 and 37). Similar to the P-WHLEDs, the CC-WHLEDs show an excellent stability in terms of both color quality—i.e., CIE: 0.32/0.34; CRI: 85; CCT: ˜5,500-6,000 K —and luminous efficiency (100 Im/W) for around 200 h. But, in stark contrast to the P-WHLEDs, a remarkable stability in terms of color and efficiency over more than 1,000 hours under ambient operation conditions was noted. This is expected as these rubbers show an excellent stability independently of the environmental conditions under both irradiation and heating treatments, as well as an enhancement of the luminescence features in the rubber material—vide supra.¹² Interestingly, while the color quality is stable over this long period of time, the luminous efficiency is immediately reduced or increased when transferring the CC-WHLED from N₂ to ambient conditions and vice versa as shown in FIG. 36. This is related to the well-known phosphorescence quenching by oxygen, which can also be circumvented by using a top isolating coating as that proposed for the P-WHLEDs. Taking the dependency of the luminous efficiency with the environment into account, this device shows extrapolated lifetimes of around 4,000 h until reaching the half of its starting maximum under ambient conditions (see FIG. 38). As such, although there is no need for encapsulation in terms of stability—vide supra, it might be advantageous for fabricating more efficient CC-WHLEDs. The latter turns more encouraging when comparing the stability of the WHLEDs provided herein with the state-of-art stability that is around a few hundreds of hours. ^{5i,k,l,o,q 8}

Conclusions

This example provides two major thrusts in the field of WHLEDs. On one hand, the ease of preparation and application of luminescent rubbers for down-converting lighting schemes has been demonstrated. Here, important assets of the present approach are i) the in-situ preparation of the rubbers without using any cross-linking and UV- or thermal-curing methods, and ii) its versatility in terms of using any kind of luminescent materials, such as fluorescent proteins,⁸ small-molecules, carbon quantum dots, polymers, and coordination complexes. Here, it has been ensured that the amount of the compounds is kept constant, but a further enhancement of the luminous efficiency should be possible if the concentration of the compounds is increased, as it will reduce the coating thickness. In this regard, the latter has been optimized to obtain a high quality white emission as shown in FIG. 33. In addition, the luminous efficiency linearly decreases upon increasing the coating thickness (see FIG. 33). On the other hand, for the first time a direct comparison of different down-converting materials has been provided, showing that under the same working conditions, WHLEDs fabricated with a down-converting rubber encapsulation based on coordination complexes bear a great potential for future breakthroughs. This is demonstrated by the unprecedented stability in terms of color quality (CRI>80) and luminous efficiency (>100 Im/W) of more than 1,000 h (extrapolated 4,000 h) independently of the environmental conditions. Equally important is the potential prospect of carbon quantum dots if the photoluminescence quantum yields are enhanced. Noteworthy, it would be interesting to determine the stability of the device under outdoor conditions—i.e., 50-70° C. and 80% moisture, however due to the sound thermal stability of all the compounds any important change to the results presented are not envisaged. More importantly, since the irradiation stability of all the down-converting compounds is slightly enhanced in the rubbers when compared to that in solution, it is safe to postulate that the stability differences between devices might be related to the intrinsic instability of the compounds.

It is important to point out that although all-inorganic white LEDs feature much higher stabilities than the WHLEDs, the CC-WHLED provided herein shows similar CRI and luminous efficiencies to those of all-inorganic white LEDs, while its stability represents a one order of magnitude enhancement compared to the state-of-the-art hybrid white LEDs. As such, it is strongly believed that the present work constitutes a landmark for future breakthroughs in the field of WHLEDs. In this regard, a future challenge is the development of down-converting encapsulation systems for high-powerful LED arrays, which hold high operation temperatures, and in particular thermally stable organic-based coatings.

### REFERENCES

1 a) Global LED Display Industry Report, 2015; b) McKinsey & Company: Lighting the way —Perspectives on the global lighting market 2012; c) US Department of Energy: Manufacturing Roadmap Solid-State Lighting Research and Development 2014.
2 a) D. Volz et al., Green Chem. 2015, 17, 1988; b) S. Reineke et al., Rev. Mod. Phys. 2013, 85, 1245.

3 a) http://www.nobelprize.org/nobel_prizes/physics/laureates/2014/press.html; b) K. Itoh et al., Jpn. J. Appl. Phys. 1991, 30, 1924; c) I. Akasaki et al., Int. Phys. Conf. Ser. 1992, 129, 851; d) S. Nakamura et al., Jpn. J. Appl. Phys. 1993, 32, L8; S. Nakamura et al., J. Appl. Phys. 1993, 74, 3911; e) S. Nakamura et al., Appl. Phys. Lett. 1994, 64, 1687; f) I. Akasaki et al., Jpn. J. Appl. Phys. 1995, 34, L1517; g) S. Nakamura et al., The Blue

Laser Diode (Springer, 1997).
4 a) J. K. Park et al., Appl. Phys. Lett. 2004, 84, 1647; b) P. Pust et al., Nature Materials, 2014, 13, 891; c) T. M. Tolhurst et al., Adv. Opt. Mater. 2015, 3, 546; d) R.-J. Xie et al., Nitride Phosphors and Solid-State Lighting; CRC Press, 2011; e) C. Che et al., J. Phys. Chem. Lett. 2011, 2, 1268; f) R. Zhang et al., Laser Photon. Rev. 2014, 8, 158.
5 a) G. Heliotis et al., Appl. Phys. Lett. 2005, 87, 103505; b) G. Heliotis et al., Adv. Mater. 2006, 18, 334; c) E. Gu et al., Appl. Phys. Lett. 2007, 90, 031116; d) I. O. Huyal et al., J. Mater. Chem. 2008, 18, 3568; e) O. Kim et al., ACS Nano 2010, 4, 3397; f) M. Stupca et al., J. Appl. Phys. 2012, 112, 074313; g) D. Ban et al., Phys. Status Solidi 2012, 9, 2594; h) W.-S. Song et al., Chem. Mater. 2012, 24, 1961; i) E.-P. Jang et al., Nanotechnology 2013, 24, 045607; j) N. J. Findlay et al., J. Mater. Chem. C 2013, 1, 2249; k) C.-F. Lai et al., Opt. Lett. 2013, 38, 4082; I) D. D. Martino et al., Sci Rep. 2014, 4, 4400; m) P.-C. Shen et al., Sci. Rep. 2014, 4, 5307; n) J. Chen et al., J. Mater. Sci. 2014, 49, 7391; o) N. J. Findlay et al., Adv. Mater. 2014, 26, 7290; p) C. Sun et al., Nanoscale 2015, 7, 12045; q) Y. Hyein et al., Nanoscale 2015, 7, 12860.
6 a) C.-Y. Sun et al., Nat. Commun. 2013, 4, 2717; b) Q. Gong et al., J. Am. Chem. Soc. 2014, 136, 16724; c) Y. Lu et al., Chem. Commun. 2014, 50, 15443; d) Y. Cui et al., Adv. Funct. Mater. 2015, 25, 4796.
7 a) H. Tetsuka et al., J. Mater. Chem. C 2015, 3, 3536; b) D. Zhou et al., ACS Appl. Mater. Interfaces 2015, 7, 15830.
8 M. D. Weber et al., Adv. Mater. 2015, 27, 5493.
9 M. Ma et al., Opt. Express 2011, 19, A11352011.
10V. Strauss et al., J. Am. Chem. Soc. 2014, 136, 17308.
11 a) Z. Yu et al., Sci. China Chem. 2013, 56, 1075; b) J. Fang et al., Adv. Fund. Mater. 2009, 19, 2671; c) A. Asadpoordarvish et al., Adv. Eng. Mater. DOI: 10.100²/adem.201500245.
12 R. D. Costa et al., lnorg. Chem. 2011, 50, 7229.

### Example 10

### Preparation of Rubber-Like Materials Using TMPE as Branched Polymer and Several Linear Polymers

To demonstrate the versatility of the approach provided herein, the preparation of the gel and the final rubber-like material according to the present invention is demonstrated by using different mass ratios of the branched polymer (in this case, trimethylolpropane ethoxylate (TMPE) with a Mₙ of 450 Da) and different linear polymers (in this case, poly(ethylene oxide) (PEO) with a Mₙ ranging from 5000 to 8000 kDa, as well as poly(2-ethyl-2-oxazoline) (PEOx) with a Mₙ of 500 kDa). As shown in Tables 6-8 below, several amounts of buffer solution (as otherwise used for the proteins) were added. Moreover, Table 9 summarizes the preparation of the gel and the final rubber-like material according to the invention demonstrated using different mass ratios of the branched polymer (in this case, trimethylolpropane ethoxylate (TMPE) with a Mₙ of 450 Da) and the linear polymer (in this case, poly(ethylene oxide) (PEO) with a Mₙ of 5000 kDa) with an aqueous saturated PEDOT:PSS solution. The rubber formation is performed as described in Example 1.

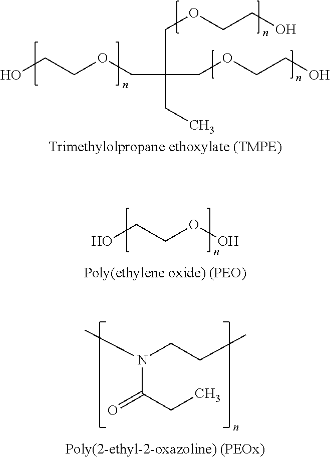

TABLE 6
Preparation of rubber-like materials using TMPE as branched
polymer and I-PEO with a Mₙ of 5000 kDa as linear polymer

| TMPE | PEO (mg) | Water-based | Gel | Rubber |
|---|---|---|---|---|
| (mg) | 5000 kDa | buffer (μL) | formation | formation |
| 60 | 1 | 150 | Low viscosity to | — |
| | | | make films | |
| 60 | 5 | 150 | Good viscosity to | yes |
| | | | make films | |
| 60 | 10 | 150 | Good viscosity to | yes |
| | | | make films | |
| 60 | 20 | 150 | Highly viscous, | yes |
| | | | not processable | |

TABLE 7
Preparation of rubber-like materials using TMPE as branched
polymer and I-PEO with a Mₙ of 8000 kDa as linear polymer

| TMPE | PEO (mg) | Water-based | Gel | Rubber |
|---|---|---|---|---|
| (mg) | 8000 kDa | buffer (μL) | formation | formation |
| 60 | 1 | 150 | Low viscosity to | yes |
| | | | make films | |
| 60 | 5 | 150 | Good viscosity to | yes |
| | | | make films | |
| 60 | 10 | 150 | Highly viscous, | yes |
| | | | but processable | |
| 60 | 20 | 150 | Highly viscous, | yes |
| | | | not processable | |

TABLE 8
Preparation of rubber-like materials using TMPE as branched
polymer and I-PEOx with a Mₙ of 500 kDa as linear polymer

| TMPE | PEOx (mg) | Water-based | Gel | Rubber |
|---|---|---|---|---|
| (mg) | 500 kDa | buffer (μL) | formation | formation |
| 60 | 10 | 150 | Low viscosity to | — |
| | | | make films | |
| 60 | 20 | 150 | Good viscosity to | yes |
| | | | make films | |
| 60 | 60 | 150 | Good viscosity to | yes |
| | | | make films | |

TABLE 9
Preparation of rubber-like materials using TMPE as branched
polymer and I-PEO with a Mₙ of 5000 kDa as linear polymer
in combination with a PEDOT:PSS solution

| TMPE | PEO (mg) | Water-based | Gel | Rubber |
|---|---|---|---|---|
| (mg) | 5000 kDa | PEDOT:PSS (μL) | formation | formation |
| 60 | 1 | 150 | Low viscosity to | — |
| | | | make films | |
| 60 | 5 | 150 | Good viscosity to | yes |
| | | | make films | |
| 60 | 10 | 150 | Good viscosity to | yes |
| | | | make films | |
| 60 | 20 | 150 | Highly viscous, | yes |
| | | | not processable | |

To determine the compatibility of the fluorescent proteins embedded into the new rubbers, the same experiments were carried out using water-based buffer solution containing a fluorescent protein (0.15 mg)—in this case mCherry—for the rubber formation as described for Example 1. A direct comparison of the luminescent features of the protein-based rubbers with those of the initial water-based buffer solutions indicates that there is no drastic denaturation or degradation of the protein during the rubber formation (see FIG. 39A) (Prodanov L et al., Biomaterials 2010, 31, 7758); the small changes noted when comparing the emission features from solution and final materials obtained with the different polymer combinations of TMPE and linear polymers are produced by small conformational changes of the protein skeleton, which do not significantly affect the binding pocket of the chromophore; note that denaturation of the luminescent proteins implies a loss of the photoluminescence features.

### Example 11

### Preparation of Rubber-Like Materials Using PEI as Branched Polymer and Several Linear Polymers

Similar to example 10, this example shows the formation of rubber-like materials by changing the branched TMPE polymer for branched PEI in combination with several linear polymers. The preparation of the gel and the final rubber-like material is demonstrated by using different mass ratios of the branched polymer (in this case, polyethylenimine (PEI, with an average M_{w} of 800 Da) and different linear polymers (in this case, poly(ethylene oxide) (PEO) with an Mₙ of 5000 and 8000 kDa, as well as poly(2-ethyl-2-oxazoline) (PEOx) with an Mₙ of 500 kDa). As shown in Tables 9-12, several amounts of buffer solution (as otherwise used for the proteins) were added. The rubber formation is performed as described in Example 1.

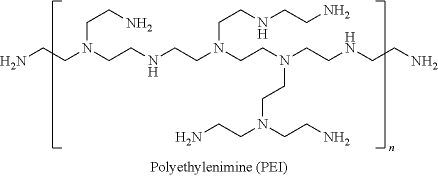

TABLE 10
Preparation of rubber-like materials using PEI as branched
polymer and PEO with a Mₙ of 5000 kDa as linear polymer

| PEI | PEO (mg) | Water-based | Gel | Rubber |
|---|---|---|---|---|
| (mg) | 5000 kDa | Buffer (μL) | formation | formation |
| 60 | 1 | 150 | Low viscosity to | — |
| | | | make films | |
| 60 | 5 | 150 | Low viscosity to | yes |
| | | | make films | |
| 60 | 10 | 150 | Low viscosity to | yes |
| | | | make films | |
| 60 | 20 | 150 | Good viscosity to | yes |
| | | | make films | |

TABLE 11

| PEI | PEO (mg) | Water-based | Gel | Rubber |
|---|---|---|---|---|
| (mg) | 8000 kDa | Buffer (μL) | formation | formation |
| 60 | 1 | 150 | Low viscosity to | — |
| | | | make films | |
| 60 | 5 | 150 | Low viscosity to | yes |
| | | | make films | |
| 60 | 10 | 150 | Good viscosity to | yes |
| | | | make films | |
| 60 | 20 | 150 | Highly viscous, | yes |
| | | | not processable | |

TABLE 12

| PEI | PEOx (mg) | Water-based | Gel | Rubber |
|---|---|---|---|---|
| (mg) | 500 kDa | Buffer (μL) | formation | formation |
| 60 | 10 | 150 | Low viscosity to | — |
| | | | make films | |
| 60 | 20 | 150 | Low viscosity to | yes |
| | | | make films | |
| 60 | 60 | 150 | Good viscosity to | yes |
| | | | make films | |

To determine the compatibility of the fluorescent proteins embedded into the new rubbers, the same experiments were carried out using water-based buffer solution containing a fluorescent protein (0.15 mg)—in this case mCherry—for the rubber formation as described for Example 1. A direct comparison of the luminescent features of the protein-based rubbers with those of the initial water-based buffer solutions indicates that there is no drastic denaturation or degradation of the protein during the rubber formation (see FIG. 39B) (Prodanov L et al., Biomaterials 2010, 31, 7758); the small changes noted when comparing the emission features from solution and final materials obtained with the different polymer combinations of PEI and linear polymers are produced by small conformational changes of the protein skeleton, which do not significantly affect the binding pocket of the chromophore; note that denaturation of the luminescent proteins implies a loss of the photoluminescence features.

### Example 12

### Preparation of Rubber-Like Materials Using TMPEMED as Branched Polymer and Several Linear Polymers

To demonstrate the versatility of the approach provided herein, the preparation of the gel and the final rubber-like material according to the present invention is demonstrated by using different mass ratios of the branched polymer (in this case, trimethylolpropane ethoxylate methyl ether diacrylate (TMPEMED) with a Mₙ of 388 Da) and different linear polymers (in this case, poly(ethylene oxide) (PEO) with a Mₙ of 5000 and 8000 kDa, as well as poly(2-ethyl-2-oxazoline) (PEOx) with a Mₙ of 500 kDa). As shown in Tables 13-15, several amounts of buffer solution (as otherwise used for the proteins) were added. The rubber formation is performed as described in Example 1.

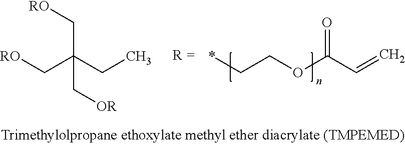

TABLE 13
Preparation of rubber-like materials using TMPEMED as branched
polymer and PEO with Mₙ of 5000 kDa as linear polymer

| TMPEMED | PEO (mg) | Water-based | Gel | Rubber |
|---|---|---|---|---|
| (mg) | 5000 kDa | Buffer (μL) | formation | formation |
| 60 | 1 | 150 | Low viscosity to | — |
| | | | make films | |
| 60 | 5 | 150 | Good viscosity to | yes |
| | | | make films | |
| 60 | 10 | 150 | Good viscosity to | yes |
| | | | make films | |
| 60 | 20 | 150 | Highly viscous, | yes |
| | | | not processable | |

TABLE 14
Preparation of rubber-like materials using TMPEMED as branched
polymer and PEO with Mₙ of 8000 kDa as linear polymer

| TMPEMED | PEO (mg) | Water-based | Gel | Rubber |
|---|---|---|---|---|
| (mg) | 8000 kDa | Buffer (μL) | formation | formation |
| 60 | 1 | 150 | Low viscosity | yes |
| | | | to make films | |
| 60 | 5 | 150 | Good viscosity | yes |
| | | | to make films | |
| 60 | 10 | 150 | Highly viscous, | yes |
| | | | but processable | |
| 60 | 20 | 150 | Highly viscous, | yes |
| | | | not processable | |

TABLE 15
Preparation of rubber-like materials using TMPEMED as branched
polymer and PEOx with Mₙ of 500 kDa as linear polymer

| TMPEMED | PEOx (mg) | Water-based | Gel | Rubber |
|---|---|---|---|---|
| (mg) | 500 kDa | Buffer (μL) | formation | formation |
| 60 | 10 | 150 | Low viscosity to | — |
| | | | make films | |
| 60 | 20 | 150 | Good viscosity to | yes |
| | | | make films | |
| 60 | 60 | 150 | Good viscosity to | yes |
| | | | make films | |

To determine the compatibility of the fluorescent proteins embedded into the new rubbers, the same experiments were carried out using water-based buffer solution containing a fluorescent protein (0.15 mg)—in this case mCherry—for the rubber formation as described for Example 1. A direct comparison of the luminescent features of the protein-based rubbers with those of the initial water-based buffer solutions indicates that there is no drastic denaturation or degradation of the protein during the rubber formation (see FIG. 39C) (Prodanov L et al., Biomaterials 2010, 31, 7758); the small changes noted when comparing the emission features from solution and final materials obtained with the different polymer combinations of PEI and linear polymers are produced by small conformational changes of the protein skeleton, which do not significantly affect the binding pocket of the chromophore; note that denaturation of the luminescent proteins implies a loss of the photoluminescence features.

### Example 13

### Preparation of Rubber-Like Materials Containing Different Luminescent Materials Immobilized Therein

The preparation of the gel and the rubber-like material according to the present invention is demonstrated using different mass ratios of the branched polymer (in this case, trimethylolpropane ethoxylate (TMPE) with an Mₙ of 450 Da) and the linear polymer (in this case, poly(ethylene oxide) (PEO) with an Mₙ of 5000 kDa), as shown in Tables 16 and 17 below. The rubber formation is performed as described in Example 1. In particular, the two polymers are mixed at different mass ratios as shown in Table 17 below. Although TMPE is a low viscous liquid, the PEO does not dissolve even at high stirring conditions. To facilitate this process, several amounts of acetonitrile or water, which is already mentioned above in Tables 1 and 2 (see Example 2), were added.

TABLE 16
Preparation of rubber-like materials using branched and linear polymers
in a mass ratio of 12:1 and different amounts of acetonitrile

| TMPE | PEO | Acetonitrile | Gel | Rubber |
|---|---|---|---|---|
| (mg) | (mg) | (μL) | formation | formation |
| 60 | 5 | 50 | Highly viscous, | — |
| | | | not processable | |
| 60 | 5 | 100 | Highly viscous, | — |
| | | | but processable | |
| 60 | 5 | 150 | Good viscosity to | yes |
| | | | make films | |
| 60 | 5 | 200 | Low viscosity to | yes |
| | | | make films | |
| 60 | 5 | 300 | Low viscosity to | yes |
| | | | make films | |
| 60 | 5 | 400 | Low viscosity to | yes |
| | | | make films | |
| 60 | 5 | 500 | Low viscosity to | yes |
| | | | make films | |

TABLE 17
Preparation of rubber-like materials using branched and
linear polymers in different mass ratios and acetonitrile

| TMPE | PEO | Acetonitrile | Gel | Rubber |
|---|---|---|---|---|
| (mg) | (mg) | (μL) | formation | formation |
| 60 | 1 | 150 | Low viscosity to | — |
| | | | make films | |
| 60 | 5 | 150 | Good viscosity to | yes |
| | | | make films | |
| 60 | 10 | 150 | Good viscosity to | yes |
| | | | make films | |
| 60 | 20 | 150 | Highly viscous, | yes |
| | | | not processable | |

As summarized in Table 16, the gel formation is the best with 150 μL acetonitrile, which indicates that they are good enough to make films by using a doctor blading technique. The next step was to determine the lowest amount of PEO. As shown in Table 17, 5-10 mg of PEO is the best amount to obtain a useful gel. As a summary, the PEO:TMPE mass ratios of 1:6 and 1:12 with a certain amount of water (150 μL) were found to be the best conditions for further processing.

The final layer is best described as rubber-like material in which the loss of most of the acetonitrile provokes the collapse of the network structure. Notably, the solvent is not recovered over weeks under ambient storage conditions (see FIG. 40). The rubber-like materials are easily peeled off from the substrate with tweezers and can be easily transferred to another substrate. The thickness of the rubber-material can be controlled either by the thickness of the stamp or by the subsequent deposition of one layer on top of another with an excellent adhesion showing roughness lower than 10%, as already explained in Examples 1 and 2.

Preparation of Rubber-Like Materials Containing Different Luminescent Compounds Embedded Therein

The formation of the luminescent gels and rubber-like materials is carried out in a similar procedure as described in Example 1. Here, commercially available luminescent compounds can be added as powder directly to the mixture of branched and linear poly(ethylene oxide) compounds—i.e., trimethylolpropane ethoxylate (TMPE) with a Mₙ of 450 Da and linear poly(ethylene oxide) (l-PEO) with Mₙ of 5×10⁶ Da, with a mass ratio of 6:1, respectively, while the solvent—i.e., either water or acetonitrile, depending on the properties (solubility, polarity, etc.) of the luminescent compounds—is added subsequently. Here, it is also proposed that the terminal hydroxyl groups provide a high compatibility with the acetonitrile solution, retaining enough solvent molecules within network. The gel network is mainly provided by the TMPE, while the I-PEO acts as a gelation agent. In the studied mass range of luminescent materials, the formation of the gel and the final rubber material are independent of the amount of embedded compounds. Furthermore, the luminescent gels and rubber-like materials exhibit similar properties as the above mentioned protein-based ones described in Examples 1 and 2 —i.e., applicable onto 3D substrates by introducing them into the gels; the thickness of the rubber-like materials can be controlled either by the thickness of the stamp or by the subsequent deposition of one layer on top of another with excellent adhesion. The presence of the luminescent compounds was corroborated by steady-state spectroscopic techniques—steady-state absorption and photoluminescence characterizations were performed by using Perkin Elmer Lambda and Fluoromax-P-spectrometer from HORIBA Jobin Yvon, respectively.

A direct comparison of the luminescent features of the final materials with those of the initial solutions indicates that there is no drastic degradation of the compounds during the formation of the luminescent rubber-like material; the small changes—i.e., maxima shift of around 5-10 nm and slight broadening of the spectrum—noted when comparing the emission features from solution and rubber-like materials are produced by small interactions of the luminescent compounds with the surrounding matrix, which do not significantly affect the luminophore.

1. A process of preparing a rubber-like material containing a protein immobilized therein, the process comprising the following steps:
(a) mixing a protein, a branched polymer and a linear polymer in an aqueous solution to form a gel; and
(b) drying the gel to obtain a rubber-like material containing the protein immobilized therein;
wherein the branched polymer comprises at least three polymeric branches bound to a central branching unit.

2. A process of preparing a gel, the process comprising:
(a) mixing a protein, a branched polymer and a linear polymer in an aqueous solution to form a gel;
wherein the branched polymer comprises at least three polymeric branches bound to a central branching unit.

3. The process of claim 1 or 2, wherein said central branching unit comprised in the branched polymer is a C₁₋₂₀ hydrocarbon moiety which is substituted with 3 to 8 substituent groups, wherein said substituent groups are each independently selected from hydroxy, carboxy and amino, optionally wherein one or more carbon atoms comprised in said C₁₋₂₀ hydrocarbon moiety are each independently replaced by an oxygen atom, a nitrogen atom or a sulfur atom, and further wherein each of the at least three polymeric branches is bound to one of the substituent groups of the C₁₋₂₀ hydrocarbon moiety.

4. The process of any one of claims 1 to 3, wherein said central branching unit comprised in the branched polymer is selected from a trimethylolpropane moiety, a trimethylolethane moiety, a trimethylolmethane moiety, a glycerol moiety, a pentaerythritol moiety, a pentaerythrithiol moiety, a diglycerol moiety, a triglycerol moiety, a dipentaerythritol moiety, a tetraglycerol moiety, a pentaglycerol moiety, a tripentaerythritol moiety, a hexaglycerol moiety, a trimethanolamine moiety, a triethanolamine moiety, a triisopropanolamine moiety, a propane-1,2,3-tricarboxylic acid moiety, a citric acid moiety, an isocitric acid moiety, a trimesic acid moiety, a 1,1,1-tris(aminomethyl)propane moiety, a 1,1,1-tris(aminomethyl)ethane moiety, a tris(aminomethyl)methane moiety, a propane-1,2,3-triamine moiety, a tris(2-aminoethyl)amine moiety, and a tris(carboxymethyl)ethylenediamine moiety.

5. The process of any one of claims 1 to 4, wherein said at least three polymeric branches comprised in the branched polymer are each independently a poly(alkylene oxide) having a terminal —OH, —OR, —O—CO—R, —CO—O—R, or —CO-—(R)—R group, wherein each R is independently C₁₋₅ alkyl or C₂₋₅ alkenyl.

6. The process of any one of claims 1 to 5, wherein the branched polymer has 3 polymeric branches bound to the central branching unit, wherein said central branching unit comprised in the branched polymer is a trimethylolpropane moiety, wherein said polymeric branches comprised in the branched polymer are each independently a poly(alkylene oxide) having a terminal —OH, —OR or —O—CO—R group wherein each R is independently C₁₋₅ alkyl or C₂₋₅ alkenyl, and further wherein the linear polymer is a poly(alkylene oxide) having a terminal group at each of its two ends which is selected independently from —OH, —OR and —O—CO—R wherein each R is independently C₁₋₅ alkyl.

7. The process of any one of claims 1 to 6, wherein the branched polymer is a trimethylolpropane ethoxylate.

8. The process of any one of claims 1 to 7, wherein the linear polymer is a poly(ethylene oxide) having a terminal —OH group at each of its two ends.

9. The process of any one of claims 1 to 8, wherein the branched polymer is a trimethylolpropane ethoxylate, and wherein the linear polymer is a poly(ethylene oxide) having a terminal —OH group at each of its two ends.

10. The process of any one of claims 1 to 9, wherein the protein is a luminescent protein or an enzyme.

11. The process of any one of claims 1 to 10, wherein the process does not comprise any step of covalently crosslinking the polymers that are mixed in step (a).

12. A rubber-like material containing a protein immobilized therein, which is obtainable by the process of claim 1 or any one of its dependent claims 3 to 11.

13. The rubber-like material of claim 12, wherein said rubber-like material is obtainable by the process of claim 9.

14. A rubber-like material containing a protein immobilized therein, wherein the rubber-like material comprises a branched polymer and a linear polymer, and wherein the branched polymer comprises at least three polymeric branches bound to a central branching unit.

15. The rubber-like material of claim 14, wherein the branched polymer is a trimethylolpropane ethoxylate, and wherein the linear polymer is a poly(ethylene oxide) having a terminal —OH group at each of its two ends.

16. A gel which is obtainable by the process of claim 2 or any one of its dependent claims 3 to 11.

17. A gel comprising a protein, a branched polymer and a linear polymer, wherein the branched polymer comprises at least three polymeric branches bound to a central branching unit.

18. Use of the rubber-like material of any one of claims 12 to 15 as a down-converting material for a hybrid light-emitting diode, wherein the protein immobilized in the rubber-like material is a luminescent protein.

19. A hybrid light-emitting diode comprising a light-emitting diode and a coating, wherein the coating contains one or more layers of a rubber-like material as defined in any one of claims 12 to 15.

20. A diagnostic device or kit comprising the rubber-like material of any one of claims 12 to 15.


SEQUENCE LISTING (1 sequence)

SEQ ID NO: 1 - protein, 7 aa, Artificial Sequence
  GS Linker
GSGSGSG